SEQUENCE LISTING (95 sequences)

SEQ ID NO: 1 - DNA, 28 bp, artificial
  synthetic oligonucleotide primer
ttactagtatcatgccaacccttttcgc

SEQ ID NO: 2 - DNA, 26 bp, artificial
  synthetic oligonucleotide primer
ttggtctccatcggataattgatgcc

SEQ ID NO: 3 - DNA, 26 bp, artificial
  synthetic oligonucleotide primer
ttggtctctcgatagaagaggggatg

SEQ ID NO: 4 - DNA, 28 bp, artificial
  synthetic oligonucleotide primer
aagagctcgcactcggtgttgatacaaa

SEQ ID NO: 5 - DNA, 29 bp, artificial
  synthetic oligonucleotide primer
ttactagttttaacgaaggtgcgctaagg

SEQ ID NO: 6 - DNA, 28 bp, artificial
  synthetic oligonucleotide primer
aaggtctcctcgaacagcgcatcatcca

SEQ ID NO: 7 - DNA, 28 bp, artificial
  synthetic oligonucleotide primer
ttggtctcatcgagcttgatcaatcttt

SEQ ID NO: 8 - DNA, 28 bp, artificial
  synthetic oligonucleotide primer
aagagctccagcttcagcttacctaaac

SEQ ID NO: 9 - DNA, 33 bp, artificial
  synthetic oligonucleotide primer
actagtgcactcaagtcgcagatattgttcgca

SEQ ID NO: 10 - DNA, 27 bp, artificial
  synthetic oligonucleotide primer
ggatccaccaacgatggcaatgggcat

SEQ ID NO: 11 - DNA, 28 bp, artificial
  synthetic oligonucleotide primer
ggatccagtaagacgcttaaccaagcat

SEQ ID NO: 12 - DNA, 34 bp, artificial
  synthetic oligonucleotide primer
gggcccggtcaatgaatcaatcagttgcaacaac

SEQ ID NO: 13 - DNA, 38 bp, artificial
  synthetic oligonucleotide primer
actagtgattacccatatcaagcactttatgactgaga

SEQ ID NO: 14 - DNA, 35 bp, artificial
  synthetic oligonucleotide primer
gagctcttgaatgcaatgggataatgtttcatccc

SEQ ID NO: 15 - DNA, 30 bp, artificial
  synthetic oligonucleotide primer
gggcccatgagccataccccttcacagcct

SEQ ID NO: 16 - DNA, 35 bp, artificial
  synthetic oligonucleotide primer
actagttaatgcggcatgtgcgattgggttgagtg

SEQ ID NO: 17 - DNA, 31 bp, artificial
  synthetic oligonucleotide primer
actagtcccccttttgcctgagccttggcgc

SEQ ID NO: 18 - DNA, 32 bp, artificial
  synthetic oligonucleotide primer
gagctcgaagatcatcgctgtccgtcgcgagc

SEQ ID NO: 19 - DNA, 32 bp, artificial
  synthetic oligonucleotide primer
actagttacatgtgcgtttattgcaactggcc

SEQ ID NO: 20 - DNA, 30 bp, artificial
  synthetic oligonucleotide primer
ccagagatatagaggcgcgaggcgagattc

SEQ ID NO: 21 - DNA, 31 bp, artificial
  synthetic oligonucleotide primer
ggtctcatggcacacgatcaaggctttttac

SEQ ID NO: 22 - DNA, 28 bp, artificial
  synthetic oligonucleotide primer
ggatccccaacaaatcagtgtcggcacc

SEQ ID NO: 23 - DNA, 32 bp, artificial; identical to SEQ ID NO: 19
  synthetic oligonucleotide primer

SEQ ID NO: 24 - DNA, 35 bp, artificial
  synthetic oligonucleotide primer
gagctcgttaaagcatcggctaaggcagataacaa

SEQ ID NO: 25 - protein, 9 aa, Xanthomonas campestris
NACLAFING

SEQ ID NO: 26 - protein, 9 aa, Micrococcus luteus
NACLGFVNG

SEQ ID NO: 27 - protein, 9 aa, Zoogloea ramigera
QLCGSGLRA

SEQ ID NO: 28 - protein, 9 aa, Homo sapiens
EACYAATPA

SEQ ID NO: 29 - protein, 9 aa, Escherichia coli
AACAGFTYA

SEQ ID NO: 30 - protein, 9 aa, mycobacterium
TACSSSLVA

SEQ ID NO: 31 - DNA, 37 bp, artificial
  synthetic oligonucleotide primer
catattatcgatatgctattcaggcatgtcatgatcg

SEQ ID NO: 32 - DNA, 38 bp, artificial
  synthetic oligonucleotide primer
caatatctcgagtcaccacgtcacactcatcattgaac

SEQ ID NO: 33 - DNA, 37 bp, artificial
  synthetic oligonucleotide primer
atctatcgataacctcgatgctcttcaagaatgtctc

SEQ ID NO: 34 - DNA, 36 bp, artificial
  synthetic oligonucleotide primer
cgatctcgaggaagatcatcgctgtccgtcgcgagc

SEQ ID NO: 35 - DNA, 38 bp, artificial
  synthetic oligonucleotide primer
attaatggcgccatgctcttccagaatgtctccatcgc

SEQ ID NO: 36 - DNA, 36 bp, artificial
  synthetic oligonucleotide primer
aatattgggccctcaccaaaccacttcggccatcga

SEQ ID NO: 37 - DNA, 36 bp, artificial
  synthetic oligonucleotide primer
aatattggcgccatgtctggtaacgctaaattcact

SEQ ID NO: 38 - DNA, 36 bp, artificial
  synthetic oligonucleotide primer
ttaatactcgagctaccaagcgatttctaaagccat

SEQ ID NO: 39 - DNA, 36 bp, artificial
  synthetic oligonucleotide primer
aatattggcgccatgttattccaaaacgtttctatc

SEQ ID NO: 40 - DNA, 38 bp, artificial
  synthetic oligonucleotide primer
ttaatactcgagctaccaaacaacttcagccatagaac

SEQ ID NO: 41 - DNA, 36 bp, artificial
  synthetic oligonucleotide primer
aatattggcgccatgttattcaacaacgtttctatc

SEQ ID NO: 42 - DNA, 36 bp, artificial
  synthetic oligonucleotide primer
aatattggcgccatgcgtttcgctaacgtttctatc

SEQ ID NO: 43 - DNA, 38 bp, artificial
  synthetic oligonucleotide primer
ttaatactcgagctaccaaacaacttcagccatagcac

SEQ ID NO: 44 - DNA, 36 bp, artificial
  synthetic oligonucleotide primer
aatattggcgccatgcacttcgaatctgttgttatc

SEQ ID NO: 45 - DNA, 37 bp, artificial
  synthetic oligonucleotide primer
ttaatactcgagctaccaaacaacttcagccatagca

SEQ ID NO: 46 - protein, 34 aa, Shewanella oneidensis
  NON_CONS (9)..(10)
  NON_CONS (18)..(19)
  NON_CONS (25)..(26)
  NON_CONS (30)..(31)
GAVVYTGVCACLGVLSGIDKVICHQLLGNMGSGL

SEQ ID NO: 47 - protein, 35 aa, Escherichia coli
  NON_CONS (10)..(11)
  NON_CONS (19)..(20)
  NON_CONS (26)..(27)
  NON_CONS (31)..(32)
GLIVVATTSAACAGFTYALDWLVPHQRHGNTGGGF

SEQ ID NO: 48 - protein, 35 aa, Staphylococcus aureus
  NON_CONS (10)..(11)
  NON_CONS (19)..(20)
  NON_CONS (26)..(27)
  NON_CONS (31)..(32)
GMVIVATESAACYAATPAIASLCFHVYVGNIGSGS

SEQ ID NO: 49 - protein, 34 aa, Medicago sativa
  NON_CONS (10)..(11)
  NON_CONS (19)..(20)
  NON_CONS (25)..(26)
  NON_CONS (30)..(31)
THLIVCTTSGGCFAGGTVLFWIAHPEYGNMGPGL

SEQ ID NO: 50 - protein, 23 aa, Shewanella oneidensis
  NON_CONS (10)..(11)
  NON_CONS (15)..(16)
ITLVVHDWGGGLQDFDCGHYILE

SEQ ID NO: 51 - protein, 23 aa, Xanthobacter autotrophicus
  NON_CONS (10)..(11)
  NON_CONS (15)..(16)
ITLVVQDWGGGMKDLDAGHFVQE

SEQ ID NO: 52 - protein, 23 aa, Agrobacterium radiobacter
  NON_CONS (10)..(11)
  NON_CONS (15)..(16)
AYVVGHDFAAGLGDTDCGHFLMV

SEQ ID NO: 53 - protein, 23 aa, porcine
  NON_CONS (10)..(11)
  NON_CONS (15)..(16)
LTINGGSNGGADHDDKAGHGAGK

SEQ ID NO: 54 - protein, 17 aa, Shewanella oneidensis
  NON_CONS (9)..(10)
  NON_CONS (14)..(15)
TSGSTGTPKYGATEMGD

SEQ ID NO: 55 - protein, 17 aa, Brevibacillus brevis
  NON_CONS (9)..(10)
  NON_CONS (14)..(15)
TSGTTGNPKYGPTETGD

SEQ ID NO: 56 - protein, 17 aa, Saccharomyces cerevisiae
  NON_CONS (9)..(10)
  NON_CONS (14)..(15)
TSGSTGAPKYWQTETGD

SEQ ID NO: 57 - protein, 17 aa, Japanese firefly
  NON_CONS (9)..(10)
  NON_CONS (14)..(15)
SSGSTGLPKYGLTETGD

SEQ ID NO: 58 - protein, 20 aa, Shewanella oneidensis
  NON_CONS (7)..(8)
  NON_CONS (14)..(15)
GAGGFLGLVYTSTPYYAHSK

SEQ ID NO: 59 - protein, 20 aa, Homo sapiens
  NON_CONS (7)..(8)
  NON_CONS (14)..(15)
GGAGYIGVFSSSATPYGKSK

SEQ ID NO: 60 - protein, 20 aa, Escherichia coli
  NON_CONS (7)..(8)
  NON_CONS (14)..(15)
GAGAGIGLTITSMASYASSK

SEQ ID NO: 61 - protein, 20 aa, Pseudomonas fragi
  NON_CONS (7)..(8)
  NON_CONS (14)..(15)
GSTSGIGINIASAHAYVAAK

SEQ ID NO: 62 - protein, 62 aa, Shewanella oneidensis
  NON_CONS (9)..(10)
  NON_CONS (26)..(27)
  NON_CONS (35)..(36)
  NON_CONS (48)..(49)
GAVVYTGVCTAIYDISNACLGVLSGIDKVICHQVGTYQLLGNMGTVSLVSFLGIGSGLNCMM

SEQ ID NO: 63 - protein, 62 aa, Arthrobacter aurescens
  NON_CONS (9)..(10)
  NON_CONS (26)..(27)
  NON_CONS (35)..(36)
  NON_CONS (48)..(49)
GLLINTSVTAMNFDLANACLGFVNGLDRYVTHQVSTFPHWGNVGPASLVLCMGVGSGLNAGM

SEQ ID NO: 64 - protein, 62 aa, Escherichi coli
  NON_CONS (10)..(11)
  NON_CONS (27)..(28)
  NON_CONS (36)..(37)
  NON_CONS (48)..(49)
GLIVVATTSACPAFDVAAACAGFTYALDWLVPHQANLDRHGNTSAASVVLLEAFGGGFTWGS

SEQ ID NO: 65 - protein, 62 aa, Staphylococcus aureus
  NON_CONS (10)..(11)
  NON_CONS (27)..(28)
  NON_CONS (36)..(37)
  NON_CONS (48)..(49)
GMVIVATESAARCFEMKEACYAATPAIASLCFHVPFNRYVGNIYTGSLIGLFSYGSGSVGEF

SEQ ID NO: 66 - protein, 62 aa, Medicago sativa
  NON_CONS (10)..(11)
  NON_CONS (28)..(29)
  NON_CONS (36)..(37)
  NON_CONS (48)..(49)
THLIVCTTSGVKRYMMYQQGCFAGGTVLFWIAHPGGLSEYGNMSSACVGVLFGFGPGLTIET

SEQ ID NO: 67 - protein, 62 aa, Gerbera hybrida
  NON_CONS (10)..(11)
  misc_feature (21)..(21): Xaa can be any naturally occurring amino acid
THLIFCTTAGVKRYMLYQQGXAAGGTVLFWMVHPGGLSEYGNLISACVGVLFGFGPGMTVET

SEQ ID NO: 68 - protein, 63 aa, Escherichi coli
  NON_CONS (10)..(11)
  NON_CONS (28)..(29)
  NON_CONS (37)..(38)
  NON_CONS (49)..(50)
VGLIAGSGGGHGVNYSISSACATSAHCIDYLNSHGTSKAMTGHSLGAAGVMSNSFGFGGTNAT

SEQ ID NO: 69 - protein, 63 aa, Mycobacterium tuberculosis
  NON_CONS (10)..(11)
  NON_CONS (28)..(29)
  NON_CONS (37)..(38)
  NON_CONS (49)..(50)
IGAAIGSGIGRGPSISIATACTSGVHNIGYVNAHGTSASMTGHLLGAAGTLCNSFGFGGTNGS

SEQ ID NO: 70 - protein, 62 aa, Zoogloea ramigera
  NON_CONS (10)..(11)
  misc_feature (20)..(20): Xaa can be any naturally occurring amino acid
NEVILGQVLPATAWGMNQLXGSGLRAVDLVEANEAFAIAIGHPIGASGGLATLCIGGGMGVA

SEQ ID NO: 71 - protein, 27 aa, Shewanella oneidensis
  NON_CONS (10)..(11)
  NON_CONS (19)..(20)
ITLVVHDWGGICWGLQDFVDCGHYILE

SEQ ID NO: 72 - protein, 27 aa, Arthrobacter aurescens
  NON_CONS (10)..(11)
  NON_CONS (19)..(20)
VVTVGHDWGGMLWGPTDPIGAGHLVGE

SEQ ID NO: 73 - protein, 27 aa, Xanthobacter autotrophicus
  NON_CONS (10)..(11)
  NON_CONS (19)..(20)
ITLVVQDWGGMAIGMKDLLDAGHFVQE

SEQ ID NO: 74 - protein, 27 aa, Agrobacterium radiobacter
  NON_CONS (10)..(11)
  NON_CONS (19)..(20)
AYVVGHDFAAMIWGLGDTCDCGHFLMV

SEQ ID NO: 75 - protein, 27 aa, porcine
  NON_CONS (10)..(11)
  NON_CONS (19)..(20)
LTINGGSNGGLLTADHDDRKAGHGAGK

SEQ ID NO: 76 - protein, 27 aa, Homo sapiens
  NON_CONS (10)..(11)
  NON_CONS (19)..(20)
LHYVGHSQGTVWNGGKDLLFYNHLDFI

SEQ ID NO: 77 - protein, 27 aa, Bacillus subtilis
  NON_CONS (10)..(11)
  NON_CONS (19)..(20)
VVVQGESGGGVAVNELDPLGLVHGADV

SEQ ID NO: 78 - protein, 27 aa, potato
  NON_CONS (10)..(11)
  NON_CONS (19)..(20)
VFVVAHDWGAFIVGEFDLVGAAHFVSQ

SEQ ID NO: 79 - protein, 17 aa, Arthrobacter aurescens
  NON_CONS (9)..(10)
  NON_CONS (14)..(15)
TSGSTGPAKYGMTETGD

SEQ ID NO: 80 - protein, 17 aa, Burkhodleria xenovorans
  NON_CONS (9)..(10)
  NON_CONS (14)..(15)
SSGSTGKPKIGSTESGD

SEQ ID NO: 81 - protein, 20 aa, Arthrobacter aurescens
  NON_CONS (7)..(8)
  NON_CONS (14)..(15)
GASGLLGVVYVSSPDYARTK

SEQ ID NO: 82 - protein, 20 aa, Comamonas testosteroni
  NON_CONS (7)..(8)
  NON_CONS (14)..(15)
GCATGIGVVISSVAAYAGSK

SEQ ID NO: 83 - protein, 20 aa, Streptomyces exfoliatus
  NON_CONS (7)..(8)
  NON_CONS (14)..(15)
GGARGLGVNISSAASYGASK

SEQ ID NO: 84 - protein, 20 aa, Aquifex aeolicus
  NON_CONS (7)..(8)
  NON_CONS (14)..(15)
GSTRGIGVNISSVVNYSTTK

SEQ ID NO: 85 - DNA, 1053 bp, Congregibacter litoralis
ggtaccgggccccatatgtctggtaacgctaaattcactttaaacgatactgctatcgtttctgttactgctcaccacgctccagaagttgttacttctgcttctttagatgatcgtatcatgcacacttacgaacgtttaggtactcaaccaggtttattagaatctttagctggtatctctgaacgtcgttggtggccagaaggtcacactttcactgaagctgctgctgaagctggtcgtaaagctatggctgctgctaacatcaaaccagaacaagttggtttattaatcgatacttctgtttctcgtgatcgtttagaaccatcttctgctgttactgttcaccacttattagatttaccatcttcttgtttaaacttcgatatggctaacgcttgtttaggtttcatgaacgctatgcaagttgctggtatgatgttagattctcgtcaaatcgatttcgctttaatcgttgatggtgaaggttctcgtcaaccacaagaaaaaactttagaacgtttagcttctgatgaagctactgttgctgatttattcgctgatttcgctactttaactttaggttctggtgctgctggtatggttttaggtcgtcactctgaaaacgctggttctcacaaaatcatcggtggtatcaaccgtgctaacacttctcaccacaaattatgtgttggtactttagatcaaatgcgtactgatactgctgctttattagaagctggtttagatgtttctgaacgtgcttgggctaacgctgaagaatacggttggttagatatggatcgttacgttatccaccaaatctcttctgttcacacttctatgttatgtgaacgtttaggtatcgatgttgataaagttccattaacttacccaaaattaggtaacactggtccagctgctgttccattaactttagctcaagaatctgaatctttaaacccaggtgatcgtgttttatgtttaggtatgggttctggtatcaacgctatggctttagaaatcgcttggtagggatccactagtcccggg

SEQ ID NO: 86 - DNA, 1050 bp, Xanthomonas campestris
ggtaccgggccccatatgttattccaaaacgtttctatcgctggtttagctcacatcgatgctccacacactttaacttctaaagaaatcaacgaacgtttacaaccaacttacgatcgtttaggtatcaaaactgatgttttaggtgatgttgctggtatccacgctcgtcgtttatgggatcaagatgttcaagcttctgatgctgctactcaagctgctcgtaaagctttaatcgatgctaacatcggtatcgaaaaaatcggtttattaatcaacacttctgtttctcgtgattacttagaaccatctactgcttctatcgtttctggtaacttaggtgtttctgatcactgtatgactttcgatgttgctaacgcttgtttagctttcatcaacggtatggatatcgctgctcgtatgttagaacgtggtgaaatcgattacgctttagttgttgatggtgaaactgctaacttagtttacgaaaaaactttagaacgtatgacttctccagatgttactgaagaagaattccgtaacgaattagctgctttaactttaggttgtggtgctgctgctatggttatggctcgttctgaattagttccagatgctccacgttacaaaggtggtgttactcgttctgctactgaatggaacaaattatgtcgtggtaacttagatcgtatggttactgatactcgtttattattaatcgaaggtatcaaattagctcaaaaaactttcgttgctgctaaacaagttttaggttgggctgttgaagaattagatcaattcgttatccaccaagtttctcgtccacacactgctgctttcgttaaatctttcggtatcgatccagctaaagttatgactatcttcggtgaacacggtaacatcggtccagcttctgttccaatcgttttatctaaattaaaagaattaggtcgtttaaaaaaaggtgatcgtatcgctttattaggtatcggttctggtttaaactgttctatggctgaagttgtttggtagggatccactagtcccggg

SEQ ID NO: 87 - DNA, 1050 bp, Xylella fastidiosa
ggtaccgggccccatatgttattcaacaacgtttctatcgctggtttagctcacatcgatgctccatgtactttaacttctcaagaaatcaacgctcgtttacaaccaatgttagaacgtatcggtatcaaatctgatgttttcgctgatatcgttggtatcaacgctcgtcgtttatggaacactaacgttcaaacttctgatgttgctactatggctgctcgtaaagctttacaagatgctggtgttgctgttgatcgtatcggtttagttgttaacacttctgtttctcgtgattacttagaaccatctactgcttctatcgtttctggtaacttaggtgttggtgaacaatgtatcgctttcgatgttgctaacgcttgtttagctttcttaaacggtatggatatcgctggtcaaatgttagaacgtggtgatatcgattacgctttagttgttaacgctgaaactgctaaccgtgtttacgaaaaaactttagaacgtatgtctgctccaggtgttactgaacaagaattccgtgaagaaatggctgctttaactttaggttgtggtgctgttgctatggttttagctcgtactgctttagttccagatgctccacaatacaaaggtggtgttactcgttctgctactgaatggaacaaattatgttgtggtaacttagatcgtatggttactgatactcgtttaatgttaatcgaaggtatcaaattagctaaaaaaactttcgttgttgctaaacaagttttaggttgggctgttgaagaattagatcaattcgttatccaccaagtttctcgtccacacactgaagctttcatcaaatctttcggtatcgatccagctaaagttatgactatcttccgtgaatacggtaacatcggtccagcttctgttccaatcgttttatctaaattaaaagaattaggtcgtttaaaaaaaggtgatcgtatcgctttattaggtatcggttctggtttaaactgttctatggctgaagttgtttggtagggatccactagtcccggg

SEQ ID NO: 88 - DNA, 1080 bp, Plesiocystis pacifica
ggtaccgggccccatatgcgtttcgctaacgtttctatctgttctgttgctcacgttgatgctccataccgtgtttcttctactgatttagaaaaccgtttagctgctccaatgcaacgtttaggtttaccaccaggtatcttagaaactttaactggtatcaaagctcgtcgtatgtggccagcttctgtttctccatctgatgctgctactttagctgctcgtcgtgctatcgctgaatctggtgttgatccagaacgtatcggtgttttaatctctacttctgtttgtcgtgatttcgttgaaccatctactgcttgtttagttcacggtaaattaggtttaccaccaacttgtttaaacttcgatgttggtaacgcttgtttaggtttcatcaacggtatggatatcatcggtaacatgatcgaacgtggtcaattagattacggtatcgttgttgatggtgaagattctcgttacgttatcgataaaactatcgaacgtttatctgctccagattctactcgtgaagatttctggtctaacttcgctactttaactttaggtggtactgctgctgctatggttttagctcgtactgatttagctcaagctttagctgaaaaacgtgctgaaggtggttactctcaccaattcttaggttctgttatcgttgctgctactcaacactctggtttatgtcgtggtcaagttgatcgtatggaaactgattctgctgaattattaactgctggtttacgtgttgctaaagaagcttggcgtgctgctcaacgtgaattcggttggactccaggtgctttagatgaatgtgttatccaccaagtttctcgtactcacactgataaattctgtgaaactttcgaattagatccagctaaattattagctacttacccagaattcggtaacgttggtccagctggtgttccaatggttttatctaaagctgcttcttctggtcgtttaggtcgtggtgatcgtgttggtttaatgggtatcggttctggtttaaactgtgctatggctgaagttgtttggtagggatccactagtcccggg

SEQ ID NO: 89 - DNA, 1050 bp, unknown
  gamma proteobacterium NOR5-3
ggtaccgggccccatatgcacttcgaatctgttgttatcttatctttagctgctgctgatgctccaatctctttaacttctaaagaaatctctcaacgtttaaaaccaactatggatcgtttaggtgttcgtgaaaacttattagaagaaatctctggtatcgcttctcgtcgtatctggaacccagaaacttctccatctgatgctgctactttagctgctgaaaaagctatccaagattctggtatcgatcgttctcgtatcggtgttatcatctctacttctgtttctcgtgatttcttagaaccatctgctgcttgtatggttcacggtaacttaggtttagcttctgattgtttaaacttcgatgttgctaacgcttgtttaggtttcttaaacggtatggatatcgctgctcgtatgatcgaacgtgaagaattagattacgctttagttgttgctggtgaatcttctcgtccattaatcgaagctactactgaacgtttattagatcaagatgttggtgctgctcaattccgtgaagaattcgcttctttaactttaggttctggtgctgctgctatgatcatgactcgtcgtgaattagctccaggtggtcacacttaccgtggttctgttactcgttctgctactcaattcaaccgtttatgtcaaggtaacatggatcgtatgcgtactgatactggtatgttattatctgctggtttagaattagctgctcaaactttcgaagcttcttgttctactttagattggtctgttgatgaaatggatcaattcatcatccaccaagtttctaaagttcacactgaatctttagttaaaactttaggtttaaacccagataaagttcacgctatctacccacacatgggtaacatcggtccagcttctgttccaatcgttttagctaaagttgaagaagctggtaaattaaaaaaaggtgatcgtatcgctttattaggtatcggttctggtttaaactgtgctatggctgaagttgtttggtagggatccactagtcccggg

SEQ ID NO: 90 - DNA, 1085 bp, Xanthomonas campestri
accatgggcagctcgcatcatcatcatcatcacagcagcggtctggtgccgcgtggtagccatatgaaaatcctggttaccggtggtggtggttttctgggccaagccctgtgtcgtggtttggtcgcacgtggtcacgaggttgtcagctttcagcgcggtgactacccggtcctgcacacgttgggcgtgggccaaatccgtggtgacctggcagaccctcaggcggtccgtcacgctttggcaggtattgatgccgtttttcacaatgccgccaaagcgggtgcatggggcagctatgattcttatcatcaagcgaatgtcgttggtactcaaaatgtcctggatgcgtgtcgcgcgaacggcgtcccgcgtttgatctacacctccaccccgtcggtgacgcatcgtgcgacgaatccggttgagggtttgggtgcggatgaagttccgtacggtgaggacttgcgtgcgccgtacgctgcgaccaaggctatcgcggagcgtgcggtcctggcagccaacgacgcgcaattggcaaccgttgcgctgcgcccacgcctgatttggggtccgggtgacaatcacctgctgccgcgtctggcagcgcgtgcccgtgccggtcgcctgcgtatggtcggtgatggcagcaacctggtggactctacctatatcgataatgcagcccaggcccacttcgatgcgtttgcgcacctggcgcctggtgcagcttgcgcgggtaaggcatacttcattagcaacggcgaaccgctgccgatgcgtgagctgctgaaccgtctgctggcagcggtggatgccccagcggtgacccgtagcctgagcttcaaaaccgcgtaccgcatcggcgctgtgtgcgaaaccctgtggccgctgctgcgcctgccgggtgaggttccgctgacgcgtttcttggttgaacagctgtgcactccgcactggtacagcatggaaccagcacgtcgcgacttcggctatgttccgcagatttctatcgaggaaggcctgcagcgtttgcgttccagcagcagccgcgacattagcattacgcgctgactcgaggatcc

SEQ ID NO: 91 - DNA, 1073 bp, Xylella fastidiosa
accatgggtagctcgcatcatcatcatcatcacagcagcggcctggtgcctcgcggcagccatatgcgtattctggtcacgggcggtagcggtttcctgggtgaggccctgtgtcgtggcctgttgaaacgcggttaccaggtggtgagctttcagcgtagccactatcaagcattgcaggccctgggtgttgtccaaatttgtggcgatttgtccgatttccacgccgtccgtcacgcggttcgtggtgtcgacgcggtttttcacaacgcggcaaaagttggtgcgtggggctcttatacgtcctatcaccagattaacgttatcggcacgcaacatgtcctggacgcgtgccgtgcagaaaacatcaataaactggtgtataccagcacccctagcgttattcatcgcagcaattacccggtcgaaggtctggacgcggaccaggttccgtacagcaacgctgtgaaagtgccgtacgcagccactaaggctatggcggaacaagcagttctggctgcaaatagcgtagatctgaccaccgtcgcactgcgtccgcgtatgatctggggtccgggcgatccgcatctgatgccgcgcttggtcgcgcgtgcgcgtgccggtcgtctgcgtctgattggtgacggtcgtaacctggtcgacagcacctacatcgataatgcagcgcaggcccacttcgacgcttttgagcacctgatgccgggtgcggcatgtgccggcaaggcttatttcatttccaatggtgagcctctgcagatgcgcgagctgatcaacaagttgctggcaacgacgaatgcaccgccggtgacccaaagcctgagcttcaagaccggctactgcattggtgccttctgcgagatgctgtggagcctgctgccgctgccgggtgaaccgctgttgacccgctttctggtcgagcaaatgagcaccccgcactggtattccatcgaaccggctaaacgtgattttggctacgttccgcgtgtcagcattgaagagggtctggtgcgtctgctgagcgagactcgcgtgacctgctgactcgaggatcc

SEQ ID NO: 92 - DNA, 1088 bp, unknown
  gamma proteobacterium NOR5
accatgggtagctcgcatcatcatcatcatcacagctcgggtctggttccgcgtggtagccatatgaagatcttggtcaccggtggcggtggtttcctgggtcaagaaatctgccacatgctgctggcgcaaggcgacgagccggtcgccttccaacgcggcgaggcacgcgcgctggcccaagcgggtatcgaagtccgtcgtggtgatattggtcgtctgcaggacgtgctggcagcggcggagggttgtgaggcggtgatccacacggcgggtaaggccggtgcatggggtgacgcgcaactgtaccgtgcggtcaacgtaagcggtacccaaaacgttctgcaagcgtgcgaagcgctgggtatccaacgcctggtttttaccagctccccgagcgtggcgcattgcggtggcgacatcgccggtggcgatgaaagcctgccgtacccgcgtcattatgcggcaccgtatccgcagactaaagctgcggcagaacagctggttatggcggcatccggcagcggtctgaataccgtgtccctgcgtccgcacctggtgtggggtcctggtgacaatcagctgttgccgcgtttggtcgagcgtgcgcgtcgtggtaccctgcgcctgccgggtgcggataagttgatcgatgcaacttacatttacaatgccgctcgcgcacacctgctggcattggcagcactggacaataacgaagcgtgtcacggtaaaacctatttcatcagcaacggtgaaccgtggccgcaagccaagattattgccgcactgctgaacgcagtgggcgtgaacgctgatatcaagccgattgcggcaggtgctgcaaaactggcgggtattttggcggagtcttggtggcgcttgagccagcgcgacgatgagcctccggtgacccgctggagcgcggaacaactggcgacggcgcactggtacgacattagcgcggcacgtaaggatttgggttacgaacctgttatcagcatggcagagggtctgaaacgtctggcccagagcgctgagaatgcccgtctggctgacgatatccagacgaaatgactcgaggatcc

SEQ ID NO: 93 - DNA, 1070 bp, Chloroflexus auranticus
accatgggtagcagccatcatcatcatcatcacagcagcggcctggtcccgcgtggcagccatatgatcgcgctggtcaccggcggtaacggtttcgttggtcgttacattgttgagcaactgctggcccgtggtgatcacgttcgtgtgattggtcgtggtgcgtatccagagctgcagtccctgggcgcagaaacctaccaggcagatctgacgttgcctgaatctgcgccggtgctggcacgtgcaatgcgtggtgtcacgaccgtgtttcacgtcgcggcaaaagcaggtctgtggggttcgtacgatgacttttaccgtgcgaacgtatccgcgacccagcgtgttgttaaagcggccatccgtgccggcgttccgaaactggtgtataccagcaccccgagcgttgttattggtcatgaagatatccatggtggcgatgaacacctgccgtatcctcgccgttacctggccccgtacccacacacgaaggcgatcgcagagcgctatgtgctggcgcaaacggacatcgcgacggtcagcctgcgtccgcacctgatttggggtccgcgcgacccgcacattctgccgcgcttgctgcgtcgtgcgcgtcgccgtatgttgttccaaatcggtgacggcacgaacctggtcgacgtctgctatgtggaaaatgcggcaaccgcgcatatccaggcggccagcgccctgaatgaacgtagcccgctgcgtggccgtgcgtacttcattggtcaggagcgtccggtgaatttgtggcaattcattggtgagatcctgaaggctgcgaattgtccgcctgttcgtggccgcatttccgcgagcgctgccaccattctggctaccggcctggaactgctgtatactatcttgcgtctgccgggtgagccgccactgactcgcctgatggtccatgagctgtctcactcccactggttcagccacgctgcggccgagcgtgactttggctacacgcctcgtattagcatcgaggaaggtctggaacgtacctttgcactgaccggctcccaaccgtgactcgaggatcc

SEQ ID NO: 94 - DNA, 1069 bp, Xanthomonas campestri
accatgggcagctcgcatcatcatcatcatcacagcagcggtctggtgccgcgtggtagccatatgaaaatcctggttaccggtggtggtggttttctgggccaagccctgtgtcgtggtttggtcgcgtggtcacgaggttgtcagctttcagcgcggtgactacccggtcctgcacacgttgggcgtgggccaaatccgtggtgacctggcagaccctcaggcggtccgtcacgctttggcaggtattgatcgtttttcacaatgccgccaaagcgggtgcatggggcagctatgattcttatcatcaagcgaatgtcgttggtactcaaaatgtcctggatgcgtgtcgcgcgaacggcgtcccgcgtttgatctaactccaccccgtcggtgacgcatcgtgcgacgaatccggttgagggtttgggtgcggatgaagttccgtacggtgaggacttgcgtgcgccgtacgctgcgaccaaggctatcgcggagcgtgcggcctgcagccaacgacgcgcaattggcaaccgttgcgctgcgcccacgcctgatttggggtccgggtgacaatcacctgctgccgcgtctggcagcgcgtgcccgtgccggtcgcctgcgtatggtcgtgaggcagcaacctggtggactctacctatatcgataatgcagcccaggcccacttcgatgcgtttgcgcacctggcgcctggtgcagcttgcgcgggtaaggcatacttcattagcaacggcgaccgctccgatgcgtgagctgctgaaccgtctgctggcagcggtggatgccccagcggtgacccgtagcctgagcttcaaaaccgcgtaccgcatcggcgctgtgtgcgaaaccctgtggccgctgcgcgcctccgggtgaggttccgctgacgcgtttcttggttgaacagctgtgcactccgcactggtacagcatggaaccagcacgtcgcgacttcggctatgttccgcagatttctatcgaggaaggctgcagcgttgcgttccagcagcagccgcgacattagcattacgcgctgactcgaggatcc

SEQ ID NO: 95 - protein, 17 aa, artificial
  consensus sequence of catalytic domains
XXGXXXXXKXXXXEXXD

(19) United States
(12) Patent Application Publication
(10) Pub. No.: US 2012/0015414 A1
(43) Pub. Date: Jan. 19, 2012

(54) Production of organic compounds

(76) Inventors: Lawrence P. Wackett, St. Paul, MN (US); Jennifer L. Seffernick, Ft. Collins, CO (US); Janice A. Frias, Minneapolis, MN (US); David J. Sukovich, San Francisco, CA (US)

(21) Appl. No.: 13/066,438

(22) Filed: Apr. 13, 2011

Related U.S. Application Data
(60) Provisional application No. 61/342,418, filed on Apr. 13, 2010.

(51) Int. Cl. C12P 7/40; C12P 5/00; C12N 1/21; C07C 11/00; C12P 1/00; C07C 49/00; C12N 15/63; C12P 7/26; C12N 15/74

(52) U.S. Cl. 435/136; 435/148; 435/166; 435/252.3; 435/471; 435/41; 568/303; 435/320.1; 585/1

(57) ABSTRACT

The present invention provides methods for the production of hydrocarbons, particularly alkanes and alkenes, using biosynthetic routes, as well as genes and enzymes involved therein.

### RELATED APPLICATIONS

The present application claims priority to U.S. Provisional Application Ser. No. 61/342,418, filed on Apr. 13, 2010, the entirety of which is incorporated herein by reference.

### STATEMENT OF GOVERNMENT RIGHTS

This work was supported in part by the Initiative for Renewable Energy and the Environment under Grant Number LG-B 13. The government may have certain rights in the invention.

### BACKGROUND

The global economy and contemporary life is dependent on hydrocarbon fuels and feedstocks. Hydrocarbons come from petroleum that has been formed by diagenesis (“slow burning”) of biological material in sediments over hundreds of millions of years. Drawbacks of petroleum use are the liberation of sulfur and nitrogen oxides that cause acid rain, respiratory diseases and other pollution problems. The location of petroleum fuel reserves, principally in the Middle East, has important geo-political implications for highly industrialized countries that do not contain petroleum reserves commensurate with their needs. And globally, known oil reserves are declining. In light of these issues, a biological process to transform renewable resources to hydrocarbons would have important implications for society.

### SUMMARY

The present invention provides methods for the production of hydrocarbons, particularly alkanes and alkenes, using biosynthetic routes, as well as genes and enzymes involved therein.

The present invention provides various methods. In one embodiment, there is provided a method of producing a ketone, the method comprising: providing one or more fatty acids; providing one or more modified cells and/or modified organisms that produce one or more OleA proteins; providing conditions effective to produce the one or more OleA proteins; and providing conditions effective to produce one or more ketones from said one or more fatty acids in the presence of the one or more OleA proteins.

In another embodiment, there is provided a method of producing a beta-keto-acid, the method comprising: providing one or more fatty acids; providing one or more modified cells and/or modified organisms that produce one or more OleA proteins; providing conditions effective to produce the one or more OleA proteins; and providing conditions effective to produce one or more beta-keto-acids from said one or more fatty acids in the presence of the one or more OleA proteins.

In another embodiment, there is provided a method of producing a ketone, the method comprising: providing one or more fatty acids; providing one or more isolated and purified OleA proteins; and combining the fatty acids with the isolated and purified OleA proteins under conditions effective to produce one or more ketones from said one or more fatty acids.

In another embodiment, there is provided a method of producing a beta-keto-acid, the method comprising: providing one or more fatty acids; providing one or more isolated and purified OleA proteins; and combining the fatty acids with the isolated and purified OleA proteins under conditions effective to produce one or more beta-keto-acids from said one or more fatty acids.

In another embodiment, there is provided a method of producing a hydrocarbon, the method comprising: providing one or more modified cells and/or modified organisms that produce one or more fatty acids and produce at least one OleA protein, at least one OleC protein, and at least one OleD protein; providing conditions effective to produce at least one OleA protein, at least one oleC protein, and at least one OleD protein; and providing conditions effective to produce one or more hydrocarbons from said one or more fatty acids in the presence of at least one OleA protein, at least one OleC protein, and at least one OleD protein, whether said proteins function in one reaction volume or not (preferably, they function simultaneously).

In another embodiment, there is provided a method of producing a hydrocarbon, the method comprising: providing one or more fatty acids; providing an isolated and purified OleA protein, an isolated and purified OleC protein, and an isolated and purified OleD protein; and providing conditions effective to produce one or more hydrocarbons from said one or more fatty acids in the presence of at least one OleA protein, at least one OleC protein, and at least one OleD protein, whether said proteins function in one reaction volume or not (preferably, they function simultaneously).

The present invention also provides modified bacterial organisms (i.e., altered relative wild-type organisms that are found in nature). In one embodiment such modified bacterial organism has altered hydrocarbon production relative to the wild-type bacterial organism. In another embodiment, such modified bacterial organism has altered ketone production relative to a corresponding unmodified bacterial organism.

In another embodiment, the present invention provides a method of modifying a bacterial organism to produce altered hydrocarbon production relative to the wild-type bacterial organism, the method comprising: removing genomic nucleic acid that encodes OleA, OleB, OleC, or OleD proteins; and inserting nucleic acid that encodes a heterologous protein having fatty acyl condensase function.

In another embodiment, the present invention provides a method of controlling the synthesis of an unsaturated hydrocarbon, the method comprising: providing a modified bacterial organism as disclosed herein; and culturing the modified bacterial organism under conditions effective to produce one or more unsaturated hydrocarbons.

In another embodiment, the present invention provides a method of controlling the synthesis of a ketone, the method comprising: providing a modified bacterial organism as disclosed herein; and culturing the modified bacterial organism under conditions effective to produce one or more ketones.

In another embodiment, the present invention provides a method of controlling the synthesis of an energy storage molecule, the method comprising: providing a modified bacterial organism as disclosed herein; and culturing the modified bacterial organism under conditions effective to produce one or more energy storage molecules.

The present invention also provides a hydrocarbon mixture and ketone mixtures produced by methods described herein.

In other embodiments are provided an isolated and purified nucleic acid construct comprising nucleic acids encoding an oleA protein, a vector comprising said isolated and purified nucleic acid, as well as a cell comprising said vector.

The present invention also provides a method of extracting a mixture of ketones from a biological culture comprising: providing a culture comprising a modified bacterial organism disclosed herein; growing the culture under conditions wherein said ketones are produced in said culture; preparing an organic extract from said culture; and purifying ketones from said extract; thereby producing an extract containing a mixture of ketones.

The terms “comprises” and variations thereof do not have a limiting meaning where these terms appear in the description and claims.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, “a,” “an,” “the,” “at least one,” and “one or more” are used interchangeably.

As used herein, the term “or” is generally employed in its sense including “and/or” unless the content clearly dictates otherwise.

The term “and/or” means one or all of the listed elements or a combination of any two or more of the listed elements.

Also herein, the recitations of numerical ranges by endpoints include the endpoints as well as all numbers subsumed within that range (e.g., 1 to 5 includes 1, 1.5, 2, 2.75, 3, 3.80, 4, 5, etc.).

The phrase “fatty acid” as used herein, unless otherwise specified, refers to a biologically produced fatty acid. These are typically carried by Coenzyme A or by an acyl carrier protein.

The phrase “isolated and purified” as it applies to nucleic acids or proteins means such nucleic acid or protein is separated from at least one component with which it is associated in nature. In the case of a polypeptide (e.g., protein) or polynucleotide (i.e., nucleic acid) that is naturally occurring, it is preferred that such polypeptide or polynucleotide be isolated or purified. Preferably, an “isolated” polypeptide or polynucleotide is one that is separate and discrete from its natural environment. Preferably, a “purified” polypeptide or polynucleotide is one that is at least 60% free, preferably 75% free, and most preferably 90% free from other components with which they are naturally associated. Polypeptides and nucleotides that are produced outside the organism in which they naturally occur, e.g., through chemical or recombinant means, are considered to be isolated and purified by definition, since they were never present in a natural environment.

The term “recombinant” or “altered” or “modified” means the molecule, nucleic acid, protein, cell, organism, etc. has been manipulated or altered in the laboratory intentionally “by the hand of man.”

“Polynucleotide” and “nucleic acid” and “nucleic acid sequence” are used interchangeably to refer to a polymeric form of nucleotides of any length, either ribonucleotides or deoxynucleotides, and includes both double- and single-stranded DNA and RNA. A polynucleotide can be linear or circular in topology. A polynucleotide can be obtained using any method, including, without limitations, common molecular cloning and chemical nucleic acid synthesis. A polynucleotide may include nucleotide sequences having different functions, including for instance coding sequences, and non-coding sequences. As used herein “coding sequence” and “coding region” and “open reading frame” are used interchangeably and refer to a polynucleotide that encodes a polypeptide when placed under the control of appropriate regulatory sequences. The boundaries of the coding region are generally determined by a translation start codon at its 5′ end and a translation stop codon at its 3′ end.

“Polypeptide” as used herein, refers to a polymer of amino acids and does not refer to a specific length of a polymer of amino acids. Thus, for example, the terms peptide, oligopeptide, protein, and enzyme are included within the definition of polypeptide, whether naturally occurring or synthetically derived, for instance, by recombinant techniques or chemically or enzymatically synthesized. This term also includes post-produceion modifications of the polypeptide, for example, glycosylations, acetylations, phosphorylations, and the like. The following abbreviations are used:

### A=Ala=Alanine T=Thr=Threonine

### V=Val=Valine C=Cys=Cysteine

### L=Leu=Leucine Y=Tyr=Tyrosine

### I=Ile=Isoleucine N=Asn=Asparagine

### P=Pro=Proline Q=Gln=Glutamine

### F=Phe=Phenylalanine D=Asp=Aspartic Acid

### W=Trp=Tryptophan E=Glu=Glutamic Acid

### M=Met=Methionine K=Lys=Lysine

### G=Gly=Glycine R=Arg=Arginine

### S=Ser=Serine H=His=Histidine

As used herein, “similarity” or “structural similarity” refers to the identity between two polypeptides. For polypeptides, structural similarity is generally determined by aligning the residues of the two polypeptides to optimize the number of identical amino acids along the lengths of their sequences; gaps in either or both sequences are permitted in making the alignment in order to optimize the number of identical amino acids, although the amino acids in each sequence must nonetheless remain in their proper order. A pair-wise comparison analysis of protein sequences can carried out using the BESTFIT algorithm in the GCG package (version 10.2, Madison Wis.). Alternatively, polypeptides may be compared using the Blastp program of the BLAST 2 search algorithm, as described by Tatiana et al., (FEMS Microbiol Lett, 174, 247-250 (1999)), and available on the world wide web at ncbi.nlm.nih.gov/BLAST/. The default values for all BLAST 2 search parameters may be used, including matrix=BLOSUM62; open gap penalty=11, extension gap penalty=1, gap x_dropoff=50, expect=10, wordsize=3, and filter on. In the comparison of two amino acid sequences, structural similarity may be referred to by percent “identity” or may be referred to by percent “similarity.” “Identity” refers to the presence of identical amino acids and “similarity” refers to the presence of not only identical amino acids but also the presence of conservative substitutions. Polypeptides of the present invention have at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% sequence similarity to a specified polypeptide.

As used herein, “sequence identity” refers to the identity between two polynucleotide sequences. Sequence identity is generally determined by aligning the residues of the two polynucleotides to optimize the number of identical nucleotides along the lengths of their sequences; gaps in either or both sequences are permitted in making the alignment in order to optimize the number of shared nucleotides, although the nucleotides in each sequence must nonetheless remain in their proper order. For example, two polynucleotide sequences can be compared using the Blastn program of the BLAST 2 search algorithm, as described by Tatiana et al., FEMS Microbiol Lett., 1999; 174: 247-250, and available on the world wide web at ncbi.nlm.nih.gov/BLAST/. The default values for all BLAST 2 search parameters may be used, including reward for match=1, penalty for mismatch=−2, open gap penalty=5, extension gap penalty=2, gap x_dropoff=50, expect=10, wordsize=11, and filter on. In some aspects of the present invention, the polynucleotides of the present invention include nucleotide sequences having a sequence identity of at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% identity to a specified nucleic acid.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. I-1. Gas chromatogram of: (A) the S. oneidensis hydrocarbon compound I (20.2 min) and (B) the product of its hydrogenation (20.8 min) that co-migrates with, and has an identical mass spectrum as, n-hentriacosane.

FIG. I-1S. Gas chromatogram of S. oneidnesis MR-1 strains: (i) wild-type, (ii) mutant lacking oleABCD, (iii) mutant lacking oleABCD complemented with pBBR1MCS2 containing oleABCD.

FIG. I-2. Nuclear Magnetic Resonance (NMR) spectrum of the hydrocarbon compound I produced by S. oneidensis strain MR-1 in deuterated chloroform (d-CHCl₃) with tetramethylsilane (TMS) as the reference standard. The fragment representing each resonance and the number of protons on integration are indicated. The structure of the compound represented by the spectrum is shown at the top.

FIG. I-3. The oleABCD genes are required for long-chain olefin production by S. oneidensis. Shown are: (a) the oleABCD and oleC regions deleted and plasmid pOleC containing the oleC gene that complemented the oleC deletion (b) DNA gel confirming gene deletion and complementation (primers used for analysis are SO1744CompF and SO1744CompR), and (c) gas chromatogram of solvent extracts from S. oneidensis: (i) wild-type, (ii) oleC deletion mutant (iii), and oleC mutant complemented with the pOleC plasmid.

FIG. I-4. Gas chromatogram (GC) of a solvent extract from recombinant S. oneidensis producing the heterologous S. maltophilia OleA protein. Compounds were identified as hydrocarbons or ketones by mass spectrometry as described in the text and are designated by the molecular formula next to each major GC peak. The asterisk indicates compound I that is endogenously produced by wild-type S. oneidensis MR-1.

FIG. I-5. Product structure and proposed pathways in S. oneidensis MR-1 wild-type and mutant strains for head-to-head hydrocarbon and ketone formation, respectively. Part (A) shows the structure of compound I identified as described in the text. Part (B) shows the proposed role of OleA in the head-to-head biosynthetic pathway. Part (C) shows a proposed pathway to ketones in the presence of the OleA protein alone.

FIG. I-6. Long-chain polyunsaturated compounds as a function of growth temperature in S. oneidensis MR-1 wild-type and an oleABCD deletion mutant. (a) Hydrocarbon (blue) and ketone (red) content at different temperatures relative to the maximum observed (at 4° C.). (b) Wild-type MR-1 (black) and the corresponding oleABCD deficient mutant (green) were downshifted from 30° C. to 4° C. and the cold temperature growth curves are shown. Experimental points are an average triplicate sampling from 6 treatments. Variation is shown as standard deviation.

FIG. II-1. Structure-based amino acid sequence alignments of OleA, OleB, OleC, OleD from S. oneidensis MR-1 (denoted MR-1 in figure) with highly conserved regions of proteins catalyzing divergent reactions from each respective superfamily. Accession numbers or pdb identifies of the proteins used are listed below: (OleA-Thiolase superfamily) OleA from Shewanella oneidensis MR-1 (gi24373309), β-ketoacyl-acyl carrier protein synthase III (FabH) from Escherichia coli (1EBL), 3-hydroxy-3-methylglutaryl-CoA synthase (HMG_CoA) from Staphylococcus aureus (1XPK), and chalcone synthase (Chalcone) from Medicago sativa (1CGZ). Blue residues indicate the glutamate that abstracts a proton to produce a carbanion for the non-decarboxylative condensation reaction, red indicates the absolutely conserved cysteine of the superfamily that forms a covalent bond with the substrate, and green residues are involved in formation of an oxyanion hole. (OleB-α/β-hydrolase superfamily) OleB from Shewanella oneidensis MR-1 (gi24373310), haloalkane dehalogenase (HAD) from Xanthobacter autotrophicus GJ10 (1B6G), epoxide hydrolase (EH) from Agrobacterium radiobacter AD1 (1EHY), and prolyloligopeptidase (Prolyl) from porcine brain (1H2W). Red residues indicate the catalytic nucleopile (Ser, Asp, or Cys in the whole superfamily), green indicates the general acid, and blue indicates the conserved histidine that activates water. (C) (OleC-AMP-dependent ligase/synthetase superfamily) OleC from Shewanella oneidensis MR-1 (gi24373311), gramicidin synthetase (Gramicidin) from Brevibacillus brevis (1AMU), acetyl-CoA synthetase (AcCoA) from Saccharomyces cervisiae (1RY2), and luciferase from the Japanese firefly (2D1Q). Red indicates absolute conservation in the three consensus regions identified by Conti, et al. ([STG]-[STG]-G-[ST]-[TSE]-[GS]-x-[PALIVM]-K, [YFW]-[GASW]-x-[TSA]-E, [STA]-[GRK]-D) (Conti et al. 1996. Structure. 4:287-98). Blue and green indicate Thr/Ser residues thought to be involved in binding the phosphoryl group in ATP and AMP. (D) (OleD—Short chain dehydrogenase/reductase superfamily) OleD from Shewanella oneidensis MR-1 (gi24373312), UDP-galactose-4-epimerase (Udp-gal-4 epim) from humans (1EK6), 7-□-hydroxysteroid dehydrogenase (7a-HOstroid DH) from Escherichia coli (1AHH), and D-3-hydroxybutyrate dehydrogenase (D-3-HObut DH) from Pseudomonas fragi (3ZTL). Blue identifies the tyrosine anion that abstracts the proton from the substrate, red is a lysine that stabilizes the tyrosine anion, green is a glycine rich region involved in cofactor NAD(P)⁺ binding, and pink is the serine that orients the substrate or stabilizes intermediates.

FIG. II-1S (3 pages). A more detailed set of alignments.

FIG. II-2. Analysis of the gene regions of: (A) S. oneidensis MR-1, (B) G. bemidijiensis Bem, and (C) G. sulfurreducans PCA. Genes denoted oleA and fabH are homologs to the oleA from S. oneidensis MR-1. A predicted oleA gene region is shown for (A) S. oneidensis and (B) G. bemidijiensis Bem, clustering with oleBCD genes. The fabH gene that is an oleA homolog with highest percent identity in G. sulfurreducans PCA fails to cluster with oleBCD homologs.

FIG. II-3. The ole gene regions of different bacteria. The gene region configuration is shown on the left, and the bacteria containing each are listed at the right. The “//” marks indicate that the genes on either side are not contiguous. Green indicates oleA, yellow oleB, red oleC, blue oleD, orange the oleBC fusion, and white indicates other genes not currently identified as being involved in hydrocarbon biosynthesis. The different parts represent: (A) The most common four contiguous gene configuration (B) The three gene cluster in which the oleB and oleC genes are in a single gene oleBC fusion. (C) Gene organization with various insertions between the identified ole genes. The white boxes indicate multiple genes that may be encoded in the same or opposite directions to the ole genes. In particular, various Xanthomonas strains have different numbers of genes identified in the indicated locations. (D) Chloroflexi that have an oligopeptidase inserted between oleB and oleC. Also the oleA homolog is located after the other genes. (E) A configuration in which pairs of genes are in different parts of the genome. (F) A configuration in which the oleA and oleB located in different parts of the genome but oleC and oleD are clustered. Note: hydrocarbon production was confirmed in at least one organism in each class A-F. Identifiers for each of the genes are listed in Table II-1S.

FIG. II-4. Gas chromatograms of extracts from different bacteria containing ole genes identified by bioinformatics. Bacteria were extracted and extracts analyzed by GC-MS as described in the Methods section. The major products are labeled with their chemical formulas. No hydrocarbon peaks were identified beyond the elution range shown.

FIG. II-5. Gas chromatograms of extracts from wild-type and mutant S. oneidensis strains with and without the oleA gene from S. maltophilia. Extracts are from the following strains: (A) S. oneidensis MR-1 wildtype, (B) S. oneidensis MR-1 wildtype with S. maltophilia oleA, (C) S. oneidensis ΔoleA, (D) S. oneidensis ΔoleA with S. maltophilia oleA, and (E) S. oneidensis ΔoleABCD with S. maltophilia oleA.

FIG. II-6. Network protein sequence clusters for (A) OleA, (B) OleB, (C)OleC, and (D) OleD are shown. The nodes represent protein sequences, and the edges represent a blast linkage that connects the two proteins with an e-score better than e⁻⁷³. The nodes are numbered to identify the organism from which each Ole protein derived. The organism names and number identifiers for each sequence are listed in the Examples Section. The nodes are colored to reflect the type of hydrocarbon produced by that organism: white indicates a C₃₁H₄₆ nonaene product; dark grey indicates diene, triene, or tetraene products; and light grey indicates monoene products.

FIG. II-6S (4 pages). Additional network diagrams that depict divergence of the clusters.

FIG. II-7. Parallel biological reaction sequences. At left, is the known reaction sequence leading to ketones in human live. At right, is the proposed reaction sequence leading to ketones in bacteria producing OleA.

FIG. III-1. Fundamentally different condensation mechanisms have been proposed for OleA:
(A) decarboxylative condensation between β-keto ester and an acyl thioester (Beller, et al), or
(B) non-decarboxylative condensation between two acyl thioesters.

FIG. III-1S (2 pages). oleA genes (as ordered from DNA 2.0 in cloning vectors).

FIG. III-2. SDS-PAGE gel showing: (A) standard molecular weight markers, and (B) purified OleA.

FIG. III-2S (2 pages). oleD genes (as ordered through DNA 2.0 in PJproduce produceion vectors).

FIG. III-3. OleA reaction products with [¹⁴C]-myristoyl-CoA as the substrate. (A) HPLC profile showing radioactive peaks. (Inset) Plot of the radioactivity detected in product 1 and product 2 over the course of 6 hours when a reaction mixture was incubated at room temperature. (B) Schematic of the reactions leading to the formation of product 1 and product 2.

FIG. III-4. Mass spectra for products from the reaction of diazomethane with: (A) product 1 (40 min retention time) from the reaction of OleA with 2-myristoylmyristic acid, and (B) synthetic 2-myristylmyristic acid.

FIG. III-5. Gas chromatogram with mass detector showing products observed on co-incubations with OleA and OleC (foreground trace) and OleA, OleC and OleD (background trace).

FIG. III-6. Gas chromatogram and accompanying mass spectra of peaks eluting between 20.3 and 20.4 minutes from:
(A) extract of reaction mixture containing OleC and OleD incubated with 2-myristoyl myristic acid;
(B) extract of reaction mixture containing OleC and OleD incubated with 14-heptacosanone; and
(C) extract of reaction mixture containing OleC incubated with 14-heptacosanone.

FIG. III-7. Proposed reaction cycle for OleA. The top of the cycle (A) shows the resting enzyme that reacts with an acyl-CoA to start the reaction cycle. CoASC(O)CH₂R₁ and CoASC(O)CH₂R₂ represent the first and second reacting acyl-Coenzyme A, respectively. B: attached by a line to Enz represents an enzyme base. The products of the reaction, 2 molecules of Coenzyme A (CoASH) and a b-keto acid, are highlighted by boxes.

FIG. IV-1. Information regarding X. campestris OleA obtained from E. coli.

FIG. V-1. GC results for a solvent extract from a wildtype C. aurantiacus culture. Compounds were identified as hydrocarbons or ketones by mass spectrometry.

FIG. V-2. GC results for a solvent extract from a recombinant S. oneidensis ΔoleABCD producing the heterologous C. aurantiacus OleA protein. Compounds were identified as hydrocarbons or ketones by mass spectrometry.

FIG. V-3. GC results for a solvent extract from wildtype Ralstonia eutropha and recombinant R. eutropha producing the heterologous C. aurantiacus OleA protein. Compounds were identified as ketones by mass spectrometry.

FIG. VI-1. Photomicrograph of mixed cultures of Synechococcus and Shewanella. The large, bright cells are Synechococcus and the small, dimmer cells are Shewanella.

FIG. VIII-1. Metabolic network analysis of Shewanella Oneidensis MR-1 showing points of modification to increase fatty acid and ketone and hydrocarbon formation.

FIG. VIII-2. Relative hydrocarbon production of different modified Shewanella oneidensis strains.

FIG. IX-1. Gas chromatograms of the extracts shown with the chain length of the ketones produced by E. coli containing OleA from Xanthomonas.

### DETAILED DESCRIPTION OF ILLUSTRATIVE EMBODIMENTS

The present invention provides methods for the production of organic compounds such as hydrocarbons and ketones, using biosynthetic routes, as well as materials involved therein.

Currently, there is industrial interest in non-gaseous microbial hydrocarbons for specialty chemical applications and, more recently, as high-energy biofuels. While many details remain to be revealed, there appear to be several different pathways by which microbes biosynthesize long-chain hydrocarbons. The most studied of the pathways involves the condensation of isoprene units to generate hydrocarbons with a multiple of five carbon atoms (C₁₀, C₁₅, C₂₀, etc.). A more obscure biosynthetic route is a reported decarbonylation of fatty aldehydes to generate a Cₙ₋₁ hydrocarbon chain. The latter offers a clean route to diesel fuels from fatty acids.

Certain microbes also make a distinctly different class of long chain hydrocarbons, generally C₂₅-C₃₃ in chain length, that contain a double bond near the middle of the chain. These long-chain olefinic hydrocarbons are thought to derive from processes different than isoprene condensation and decarbonylation mechanisms. This class of hydrocarbons has been shown by carbon-14 labeling studies to derive from fatty acids. The process has become known as head-to-head hydrocarbon biosynthesis. In this pathway, the hydrocarbons are described to arise from the formation of a carbon-to-carbon bond between the carboxyl carbon of one fatty acid and the α-carbon of another fatty acid. This condensation results in a particular type of hydrocarbon with chain lengths of C₂₃-C₃₃ and containing one or more double bonds, wherein one double bond involves the median carbon in the chain at the point of fatty acid condensation. An example of this overall biosynthetic pathway leading to the formation of specific C₂₉ olefinic hydrocarbon isomers from fatty acid precursors has been demonstrated in vivo and in vitro. In cell extracts, it has been shown that one of the fatty acid carboxyl groups is lost as carbon dioxide with the remaining carbon atoms being retained in the resultant hydrocarbon, which contains a double bond at the point of condensation.

Products of the head-to-head mechanism have been identified in gram positive bacteria such as Micrococcus luteus and Arthrobacter aurescens, and in Gram negative bacteria such as Stenotrophomonas maltophilia. Micrococcus and Arthrobacter strains produce fatty acids that are methyl branched terminally and subterminally. The long-chain olefinic hydrocarbons from those strains similarly contain a mixture of terminal and sub-terminal methyl group branching.

More recently, the genes encoding head-to-head fatty acid condensation pathway enzymes from Micrococcus luteus have been described. These are known as ole genes for the olefin products formed. Three genes from Micrococcus luteus were shown to confer on Escherichia coli the ability to make long-chain olefinic hydrocarbons. A three or four gene cluster has also been described as being involved in head-to-head hydrocarbon biosynthesis to make olefins, including homologs to ole genes in different bacteria, such as strains of Shewanella. A cluster of four genes, oleABCD, was shown to be involved in olefin biosynthesis by Shewanella oneidensis strain MR-1.

Bacteria of the genus Shewanella have been heavily studied over the last decade because they are widespread and have the ability to use a startling variety of electron acceptors for respiration. There are more than twenty completed genome sequences for Shewanella strains. The model system for studying Shewanella is S. oneidensis MR-1. The genome sequencing of S. oneidensis MR-1 was reported in 2002 and the organism has been shown to be highly amenable to genetic manipulation.

While genetic and biochemical data has provided evidence for Ole proteins producing long chain olefins in M. luteus and S. oneidensis, there are many outstanding details of the biosynthesis that remain to be elucidated. Moreover, the extent to which microbial and other species produce head-to-head olefins is unclear. International Patent Application No. WO 2008/113041 presented tables of genes homologous to the ole genes described by Beller et al. Appl. Environ. Micro. 76:1212-1223. However, the homologs identified included genes from mouse and tree frog, organisms not known to produce head-to-head hydrocarbons. Additionally, hydrocarbon biosynthetic genes from Arthrobacter sp. FB24 were claimed in International Patent Application No. WO 2008/147781 as well as International Patent Application No. WO 2008/113041, but that strain was later shown not to produce hydrocarbons under identical conditions for which other Arthrobacter strains did (Frias et al. 2009. Microbiol. 75:1774-1777).

In this context, in the present disclosure, the protein sequence families of Ole proteins and the configurations of putative ole genes within genomes were studied to identify those most likely to be involved in head-to-head hydrocarbon biosynthesis. This was followed up with experimental testing for the presence of long chain head-to-head hydrocarbons in representative bacteria from diverse phyla. This study also found that, of closely related bacteria, some produce head-to-head hydrocarbons and others do not.

A previous study of in vitro olefin biosynthesis from myristyl-CoA showed ketone and olefin biosynthesis in vitro and proposed a mechanism requiring the participation of ancillary proteins not encoded in the oleABCD gene cluster (Beller et al. Appl. Environ. Micro. 76:1212-1223). The mechanism proposed fatty acyl oxidation to generate a β-keto acid that is the substrate for the OleA protein. In fact, different mechanisms have been suggested previously for the biosynthesis of head-to-head olefins and different roles for the OleA protein have been proposed. It is not possible to deduce the olefinic biosynthetic pathway or individual reaction types based on protein sequence alignments alone because this pathway is unique, differing markedly from isoprenoid or decarbonylation hydrocarbon biosynthesis pathways. Moreover, the individual Ole proteins are each homologous to proteins that collectively catalyze diverse reactions.

In this context, the present disclosure provides: (a) a more detailed study of the Ole protein superfamilies, (b) the identification of likely olefin (ole) biosynthetic genes out of thousands of homologs, (c) evidence of experimentally tested bacteria from different Phyla for long-chain olefins, (d) insights into the role of OleA in head-to-head olefin biosynthesis, and (e) an alternative mechanism for head-to-head condensation of fatty acyl groups.

In one aspect of the present invention, Shewanella oneidensis strain MR-1 is used as a model system to investigate hydrocarbon biosynthetic genes and the possible biological function of the proteins they encode. The hydrocarbon produced by the Ole proteins in S. oneidensis MR-1 was found to be very different from hydrocarbons previously identified as deriving from a head-to-head condensation mechanism. The product was identified here as 3,6,9,12,15,19,22,25,28-hentriacontanonaene by chemical modification studies, mass spectrometry, and nuclear magnetic resonance spectroscopy. Previously, a similar polyolefin had been identified in many Antarctic bacteria. Cloning of a heterologous oleA gene into S. oneidensis MR-1 was found to produce a completely different set of products. A hydrocarbon deletion mutant showed a distinctly longer growth lag than wild-type cells when shifted to a lower temperature, suggesting that the ole genes in S. oneidensis MR-1 may aid the cells in adapting to a sudden drop in temperature.

More specifically, a polyolefinic hydrocarbon was found in non-polar extracts of Shewanella oneidensis MR-1 and identified as 3,6,9,12,15,19,22,25,28-hentriacontanonaene (I) by mass spectrometry, chemical modification, and nuclear magnetic resonance spectroscopy. Compound I was shown to be the product of a head-to-head fatty acid condensation biosynthetic pathway dependent on genes denoted as ole (olefin biosynthesis). Four ole genes were present in S. oneidensis MR-1. Deletion of the entire oleABCD gene cluster led to the complete absence of non-polar extractable products. Deletion of the oleC gene alone generated a strain that lacked compound I, but produced a structurally analogous ketone. Complementation of the oleC gene eliminated formation of the ketone and restored the biosynthesis of compound I. A recombinant S. oneidensis strain containing oleA from Stenotrophomonas maltophilia strain R551-3 produced at least 17 related long-chain compounds in addition to compound I, 13 of which were identified as ketones. A potential role for OleA in head-to-head condensation was proposed. It was further proposed that long-chain polyunsaturated compounds aid in adapting cells to a rapid drop in temperature, based on three observations. In S. oneidensis wild-type cells, the cellular concentration of polyunsaturated compounds increased significantly with decreasing growth temperature. Secondly, the oleABCD deletion strain showed a significantly longer lag phase compared to the wild-type strain when shifted to a lower temperature. Lastly, compound I has been identified in a significant number of bacteria isolated from cold environments.

Previous studies identified the oleABCD genes involved in head-to-head olefinic hydrocarbon biosynthethesis. The present study more fully defined the OleABCD protein families within the thiolase, α/β-hydrolase, AMP-dependent ligase/synthase, and short chain dehydrogenase superfamilies, respectively. Only 0.1-1% of each superfamily represent likely Ole proteins. Sequence analysis based on structural alignments and gene context was used to identify highly likely ole genes. Selected microorganisms from the Phyla Verucomicrobia, Planctomyces, and Chloroflexi, Proteobacteria, and Actinobacteria were tested experimentally and shown to produce long-chain olefinic hydrocarbons. However, different species from the same genera sometimes lack the ole genes and fail to produce olefinic hydrocarbons. Overall, only 1.9% of 3558 genomes analyzed showed clear evidence for containing ole genes. The type of olefins produced by different bacteria differed greatly with respect to the number of carbon-carbon double bonds. The greatest number of organisms surveyed biosynthesized a single long-chain olefin, 3,6,9,12,15,19,22,25,28-hentriacontanonaene, that contained nine double bonds. Xanthomonas campestris produced the greatest number of distinct olefin products, fifteen compounds ranging in length from C₂₈ to C₃₁ and containing one to three double bonds. The type of long-chain product formed was shown to be dependent on the oleA gene in experiments with Shewanella oneidensis MR-1 ole gene deletion mutants containing native or heterologous oleA genes produced in trans. A strain deleted in oleABCD and containing oleA in trans produced only ketones. Based on these observations, it was proposed that OleA catalyzes a non-decarboxylative thiolytic condensation of fatty acyl chains to generate a β-ketoacyl intermediate that can decarboxylate spontaneously to generate ketones.

In one embodiment, the present invention provides a method of producing a ketone, the method comprising: providing one or more fatty acids; providing one or more modified cells and/or modified organisms that produce one or more OleA proteins; providing conditions effective to produce the one or more OleA proteins; and providing conditions effective to produce one or more ketones from said one or more fatty acids in the presence of the one or more OleA proteins.

In another embodiment, the present invention provides a method of producing a beta-keto-acid, the method comprising: providing one or more fatty acids; providing one or more cells and/or organisms that produce one or more OleA proteins; providing conditions effective to produce the one or more OleA proteins; and providing conditions effective to produce one or more beta-keto-acids from said one or more fatty acids in the presence of the one or more OleA proteins.

In another embodiment, the present invention provides a method of producing a ketone, the method comprising: providing one or more fatty acids; providing one or more isolated and purified OleA proteins; and combining the fatty acids with the isolated and purified OleA proteins under conditions effective to produce one or more ketones from said one or more fatty acids.

In still another embodiment, the present invention provides a method of producing a beta-keto-acid, the method comprising: providing one or more fatty acids; providing one or more isolated and purified OleA proteins; and combining the fatty acids with the isolated and purified OleA proteins under conditions effective to produce one or more beta-keto-acids from said one or more fatty acids.

In another embodiment, a method of producing a hydrocarbon is provided, the method comprising: providing one or more fatty acids; providing an isolated and purified OleA protein, an isolated and purified OleC protein, and an isolated and purified OleD protein; and providing conditions effective to produce one or more hydrocarbons from said one or more fatty acids in the presence of at least one OleA protein, at least one OleC protein, and at least one OleD protein, whether said proteins function in one reaction volume (preferably, simultaneously).

In any of these methods, preferably the OleA protein is encoded by a nucleic acid having at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% sequence identity to nucleic acids identified in the following table, and the protein encoded by said nucleic acid functions as a condensase, preferably a fatty acyl condensase.

Alternatively, or additionally, in any of these methods, preferably the OleA protein has at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% similarity to proteins identified in the following table, and the protein functions as a condensase, preferably a fatty acyl condensase.

| | Protein | |
|---|---|---|
| Organism | accession | Nucleic acid accession |
| Shewanella | NP_717352 | NC_004347.1: |
| oneidensis MR-1 | | 1821611 . . . 1822660 |
| Congregibacter | ZP_01103251 | complement(NZ_AAOA01000007.1: |
| litoralis KT71 | | 57428 . . . 58447) |
| Xanthomonas | NP_635607 | NC_003902.1: 267353 . . . 268369 |
| campestris pv. | | |
| campestris str. | | |
| ATCC 33913 | | |
| Xylella fastidiosa | NP_299252 | complement(NC_002488.3: |
| 9a5c | | 1880089 . . . 1881105) |
| Plesiocystis | ZP_01906524 | complement(NZ_ABCS01000010.1: |
| pacifica SIR-1 | | 10353 . . . 11399) |
| gamma | ZP_05127044 | complement(NZ_DS999405.1: |
| proteobacterium | | 1429205 . . . 1430221) |
| NOR5-3 | | |

The information provided by the accession numbers in the above table are incorporated herein by reference.

Herein, preferred oleA proteins produced using heterologous nucleic acid. Examples of oleA nucleic acid (e.g., oleA genes) has been obtained from Stenotrophomonas and Xanthomonas bacteria, which are preferred and produced in Shewanella bacteria (better for oleA nucleic acid from Stenotrophomonas) and/or E. coli (better for oleA nucleic acid from Xanthomonas). Alternatively, oleA nucleic acid (e.g., oleA genes) has been obtained from Xylella and produced in E. coli.

The present invention also provides isolated and purified nucleic acid constructs comprising nucleic acids encoding an OleA protein (optionally with OleB proteins and/or OleC proteins and/or OleD proteins). Vectors comprising such nucleic acids, and cells comprising such vectors are also provided.

The fatty acids typically function as substrates for the bacteria. In certain embodiments, the fatty acids comprise one or more saturated or unsaturated (e.g., mono-, di-, or poly-unsaturated) fatty acids. Preferably, the fatty acids include saturated, mono-unsaturated, and di-unsaturated. The fatty acids can be produced in situ (e.g., by the organism that produces the desired protein). Alternatively, the fatty acids can be provided by an external source (e.g., to the organism that produces the desired protein). For example, for cells or organisms deficient in one or more fatty acids, fatty acids can be added to the culture. That is, it is possible to supply an unusual exogenous fatty acid which might get incorporated into final products.

When ketones are produced by methods described herein, the ketone can optionally include functional groups other than a ketone. Examples of such functional groups include alcohols, esters, ethers, phosphates, cofactors (e.g., AMP or CoA or ACP), phosphodiester, enol, thioester, thioether, phosphonate, thiol, thione, carboxylic acid, aldehyde, ketene, epoxide, or cyclic rings that result from the formation of any of these groups.

Ketones can be converted, via one step synthetic processes, to thioketones, ethers, imines, hydrzones, oximes, amines, dichlorides, dibromides, alcohols, epoxides, cyanohydrins (Smith et al. 2007. Advanced Organic Chemistry. 6^{th} Ed., Wiley Interscience, New York). Via biochemical reactions, the ketones could be converted to other functional groups, for example, alcohols, phosphate esters and esters. The alcohol would be generated by reduction by a dehydrogenase. The phosphate ester could be generated by the combined action of a dehydrogenase and a kinase using ATP as a co-substrate. The ketone could be converted to an ester via a Baeyer-Villager type monooxygenase.

Organisms described herein can include a photosynthetic organism, such as a cyanobacterium (e.g., a Synechococcus bacterium). In certain embodiments, a mixture of a photosynthetic organism and a non-photosynthetic organism can be used. For example, a mixture of organisms can include a cyanobacterium and a heterotrophy, such as a mixture of a Synechococcus bacterium (a cyanobacterium) and a Shewanella bacterium (a heterotroph). Various mixtures of one, two, three, or more organisms can be used if desired.

The present disclosure provides unexpected benefits as a result of a cyanobacterium supporting the growth of a heterotroph Shewanella. The studies were conducted with Synechococcus elongatus and Shewanella oneidensis, and the results are provided in the Examples Section. In this experiment, Shewanella were grown on medium that Synechooccus had been grown in, or in a control, media alone. The medium was devoid of nutrients unless material had been secreted by the Synechococcus (cyanobacterial) strain. That carbon must come from carbon dioxide since no other carbon was present.

A photosynthetic strain such as a Synechococcus bacterium (a cyanobacteria) that produces either lactate or sugars (glucose and fructose) can be used to secrete lactate or sugars, which are then fed to a Shewanella bacteria, containing OleA, that make hydrocarbons and ketones. The OleA can be from Stenotrophomonas maltophilia or Chloroflexus auranticus, for example, as described in the Examples Section. Other photosynthetic cyanobacteria suitable for this use include, for example strains of (genera name listed), Anaebena, Bacularia, Gleobacter, Gleocapsa, Halothece, Microcystis, Nostoc, Oscillatoria, Prochlorococcus, Prochloron, Prochlorothrix, Radiocystis, Spirulina, Synechococcus, and Synechocystis.

According to the present disclosure, mixtures of bacteria can be used to provide several benefits. In one aspect, each organism is a specialist and does its function better than any single organism alone. For example, the mixture of a photosynthetic bacterium and a heterotrophic bacterium provides for a system that captures carbon dioxide as the carbon source and makes hydrocarbons or ketones efficiently. The photosynthetic bacterium can be made to excrete organic compounds, reduced carbon sources that feed the heterotrophic bacterium and provide carbon for the production of ketones and hydrocarbons.

Another benefit of the system is the flexibility to produce hydrocarbons driven by light and photosynthesis during the day and to switch to a cheap carbon source during the dark. Thus, production can continue around the clock.

A co-culture can be better than a monoculture because two organisms may help each other by ways other than one feeding the other sugars. For example, the cyanobacterium may feed the heterotroph and the heterotroph may take away toxic species produced by the cyanobacterium. An example of such a toxic compound may be reactive oxygen species such as superoxide anion, hydrogen peroxide, other peroxides, and hydroxyl radical. Reactive oxygen species are known to be generated during photosynthesis.

In certain embodiments of methods and organisms of the present invention, one or more OleA proteins are produced in a greater amount than any Ole B, C, or D proteins, if any of these are even present in the methods or organisms. In certain embodiments, one or more OleA proteins is produced and substantially no Ole B, C, or D proteins.

In certain embodiments of the present invention, a method of producing a hydrocarbon is provided, the method comprising: providing one or more modified cells and/or modified organisms that produce one or more fatty acids and produce at least one OleA protein, at least one OleC protein, and at least one OleD protein; providing conditions effective to produce at least one OleA protein, at least one oleC protein, and at least one OleD protein; and providing conditions effective to produce one or more hydrocarbons from said one or more fatty acids in the presence of at least one OleA protein, at least one OleC protein, and at least one OleD protein, whether said proteins function in one reaction volume (preferably, simultaneously).

In such methods, the OleA protein, OleC protein, and OleD protein can be produced by the same cell or organism or by different cells or organisms.

The present invention also provides a modified bacterial organism that has altered hydrocarbon production relative to the wild-type bacterial organism. The present invention also provides a modified bacterial organism that has altered ketone production relative to the wild-type bacterial organism. Preferably, herein the modified bacterial organism produces one or more hydrocarbons and/or ketones and comprises a modified OleABCD nucleic acid region encoding the corresponding ABCD proteins. In certain embodiments, the modified bacterial organism produces substantially no OleB protein. In certain embodiments, the modified bacterial organism includes a modified OleABCD nucleic acid region that encodes OleA protein and substantially no OleB protein, OleC protein, or OleD protein.

The present invention also provides a modified bacterial organism containing substantially no genomic nucleic acid (i.e., that which has not been modified relative to that which occurs naturally in an organism) that encodes OleA, OleB, OleC, or OleD proteins (in that particular wild-type organism), and includes nucleic acid encoding a heterologous protein having condensase function, preferably fatty acyl condensase function. Such modified bacterial organisms typically further include regulatory elements to regulate produceion of the nucleic acid encoding the heterologous protein. Such regulatory elements typically include promoters, silencers, enhancers, and combinations thereof. In certain embodiments the modified bacterial organism of any one of claims 31 through 38 which is a Shewanella bacterium, in particular a Shewanella oneidensis bacterium, such as a Shewanella oneidensis strain MR-1.

The present invention also provides a method of modifying a bacterial organism to produce altered hydrocarbon production relative to the wild-type bacterial organism comprising: removing genomic nucleic acid that encodes OleA, OleB, OleC, or OleD proteins; and inserting nucleic acid that encodes a heterologous protein having fatty acyl condensase function.

The present invention also provides a method of controlling the synthesis of a saturated hydrocarbon, unsaturated hydrocarbon, a ketone, and/or other energy storage molecules, the method comprising: providing a modified bacterial organism as described herein; and culturing the modified bacterial organism under conditions effective to produce one or more saturated hydrocarbons, unsaturated hydrocarbons, and/or ketones and/or other energy storage molecules. Such methods can further involve regulating the substrate composition available to the modified bacterial organism for conversion. Such substrate composition can include one or more fatty acids (e.g., one or more saturated or unsaturated, such as mono- or poly-unsaturated, fatty acids). Preferably, such substrate composition can include one or more saturated fatty acids, mono-unsaturated fatty acids, and/or di-unsaturated fatty acids. Novel mixtures of hydrocarbons, ketones, and/or other energy storage molecules can be produced by such methods.

The synthesis of hydrocarbons, ketones, and/or other energy storage molecules can take place in either a batch or continuous process that utilize either free or immobilized biomaterial (e.g., cells and/or organisms), for example. Batch cultures can include fed-batch cultures and perfusion cultures. Free systems can utilize one or more continuously stirred-tank bioreactors. Immobilization techniques include the use of dialysis membranes, biomaterial covalently bonded to a solid support, or entrapment of biomaterials within natural or synthetic polymers.

The present invention also provides a method of extracting a mixture of ketones from a biological culture comprising: providing a culture comprising a modified bacterial organism as described herein; growing the culture under conditions wherein said ketones are produced in said culture; preparing an organic extract from said culture; and purifying ketones from said extract; thereby producing an extract containing a mixture of ketones. Separation of the products can be accomplished through any number of known separation techniques. Examples include, but are not limited to, distillation, including flash distillation, filtration (including membrane filtration), electrodialysis using bipolar membranes, and solvent extraction methods.

### Illustrative Embodiments

1. A method of producing a ketone, the method comprising:
   providing one or more fatty acids;
   providing one or more modified cells and/or modified organisms that produce one or more OleA proteins;
   providing conditions effective to produce the one or more OleA proteins; and
   providing conditions effective to produce one or more ketones from said one or more fatty acids in the presence of the one or more OleA proteins.
2. A method of producing a beta-keto-acid, the method comprising:
   providing one or more fatty acids;
   providing one or more modified cells and/or modified organisms that produce one or more OleA proteins;
   providing conditions effective to produce the one or more OleA proteins; and
   providing conditions effective to produce one or more beta-keto-acids from said one or more fatty acids in the presence of the one or more OleA proteins.
3. A method of producing a ketone, the method comprising:
   providing one or more fatty acids;
   providing one or more isolated and purified OleA proteins; and
   combining the fatty acids with the isolated and purified OleA proteins under conditions effective to produce one or more ketones from said one or more fatty acids.
4. A method of producing a beta-keto-acid, the method comprising:
   providing one or more fatty acids;
   providing one or more isolated and purified OleA proteins; and
   combining the fatty acids with the isolated and purified OleA proteins under conditions effective to produce one or more beta-keto-acids from said one or more fatty acids.
5. The method of any one of embodiments 1 through 4 wherein the OleA protein is encoded by a nucleic acid having at least 30% sequence identity to nucleic acids identified as

| | Protein | |
|---|---|---|
| Organism | accession | Nucleic acid accession |
| Shewanella | NP_717352 | NC_004347.1: |
| oneidensis MR-1 | | 1821611 . . . 1822660 |
| Congregibacter | ZP_01103251 | complement(NZ_AAOA01000007.1: |
| litoralis KT71 | | 57428 . . . 58447) |
| Xanthomonas | NP_635607 | NC_003902.1: 267353 . . . 268369 |
| campestris pv. | | |
| campestris str. | | |
| ATCC 33913 | | |
| Xylella fastidiosa | NP_299252 | complement(NC_002488.3: |
| 9a5c | | 1880089 . . . 1881105) |
| Plesiocystis | ZP_01906524 | complement(NZ_ABCS01000010.1: |
| pacifica SIR-1 | | 10353 . . . 11399) |
| gamma | ZP_05127044 | complement(NZ_DS999405.1: |
| proteobacterium | | 1429205 . . . 1430221) |
| NOR5-3 | | |

and wherein the protein encoded by said nucleic acid functions as a fatty acyl condensase.
6. The method of embodiment 5 wherein the OleA protein is encoded by a nucleic acid having at least 80% sequence identity to said nucleic acids.
7. The method of any one of embodiments 1 through 6 wherein the fatty acids comprise one or more unsaturated fatty acids (preferably, mono-unsaturated or di-unsaturated), one or more saturated fatty acids, or combinations thereof.
8. The method of embodiment 1 or embodiment 3 and embodiments 5 through 7 as they depend on embodiment 1 or embodiment 3, wherein the ketone comprises functional groups other than a ketone.
9. The method of embodiment 8 wherein the other functional groups comprise alcohols, esters, ethers, phosphates, cofactors, phosphodiester, enol, thioester, thioether, phosphonate, thiol, thione, carboxylic acid, aldehyde, ketene, epoxide, or cyclic rings that result from the formation of any of these groups.
10. The method of any one of embodiments 1 or embodiment 2 wherein the one or more cells and/or organisms produce the one or more fatty acids.
11. The method of embodiment 1 or embodiment 2 and embodiments 5 through 10 as they depend on embodiment 1 or embodiment 2, wherein the organism is a photosynthetic organism, or wherein the method further comprises providing one or more photosynthetic organisms to produce nutrients for the one or more organisms that produce one or more OleA proteins.
12. The method of embodiment 11 wherein the photosynthetic organism is a cyanobacterium.
13. The method of embodiment 12 wherein the cyanobacterium is a Synechococcus bacterium.
14. The method of embodiment 1 or embodiment 2 wherein providing conditions effective to produce the one or more OleA proteins occurs in a greater amount than any Ole B, C, or D proteins, if present.
15. The method of embodiment 14 wherein providing conditions effective to produce the one or more OleA proteins in a greater amount than Ole B, C, or D proteins comprises providing conditions effective to produce the one or more OleA proteins and substantially no Ole B, C, or D proteins.
16. The method of embodiment 1 or embodiment 2 and embodiments 5 through 13 as they depend on embodiment 1 or embodiment 2, wherein the one or more cells and/or organisms produce one or more OleA proteins and substantially no Ole B, C, or D proteins.
17. The method of embodiment 1 or embodiment 2 and embodiments 5 through 13 as they depend on embodiment 1 or embodiment 2, wherein the one or more cells and/or organisms comprises a mixture of a photosynthetic organism and a non-photosynthetic organism.
18. The method of embodiment 17 wherein the mixture of organisms comprises a cyanobacterium and a heterotroph.
19. The method of embodiment 18 wherein the mixture of organisms comprises a Synechococcus bacterium and a Shewanella bacterium.
20. A method of producing a hydrocarbon, the method comprising:
   providing one or more modified cells and/or modified organisms that produce one or more fatty acids and produce at least one OleA protein, at least one OleC protein, and at least one OleD protein;
   providing conditions effective to produce at least one OleA protein, at least one oleC protein, and at least one OleD protein; and
   providing conditions effective to produce one or more hydrocarbons from said one or more fatty acids in the presence of at least one OleA protein, at least one OleC protein, and at least one OleD protein.
21. A method of producing a hydrocarbon, the method comprising: providing one or more fatty acids;
   providing an isolated and purified OleA protein, an isolated and purified OleC protein, and an isolated and purified OleD protein; and
   providing conditions effective to produce one or more hydrocarbons from said one or more fatty acids in the presence of at least one OleA protein, at least one OleC protein, and at least one OleD protein.
22. The method of embodiment 20 wherein the OleA protein, OleC protein, and OleD protein are produced by the same cell or organism.
23. The method of any one of embodiments 20 through 22 wherein the OleA protein is encoded by a nucleic acid having at least 30% sequence identity to nucleic acids identified as

| | Protein | |
|---|---|---|
| Organism | accession | Nucleic acid accession |
| Shewanella | NP_717352 | NC_004347.1: |
| oneidensis MR-1 | | 1821611 . . . 1822660 |
| Congregibacter | ZP_01103251 | complement(NZ_AAOA01000007.1: |
| litoralis KT71 | | 57428 . . . 58447) |
| Xanthomonas | NP_635607 | NC_003902.1: 267353 . . . 268369 |
| campestris pv. | | |
| campestris str. | | |
| ATCC 33913 | | |
| Xylella fastidiosa | NP_299252 | complement(NC_002488.3: |
| 9a5c | | 1880089 . . . 1881105) |
| Plesiocystis | ZP_01906524 | complement(NZ_ABCS01000010.1: |
| pacifica SIR-1 | | 10353 . . . 11399) |
| gamma | ZP_05127044 | complement(NZ_DS999405.1: |
| proteobacterium | | 1429205 . . . 1430221) |
| NOR5-3 | | |

and wherein the protein encoded by said nucleic acid functions as a fatty acyl condensase.
24. The method of embodiment 23 wherein the OleA protein is encoded by a nucleic acid having at least 80% sequence identity to said nucleic acids.
25. The method of any one of embodiments 20 through 24 wherein the fatty acids comprise one or more unsaturated fatty acids (preferably, mono-unsaturated or di-unsaturated), one or more saturated fatty acids, or combinations thereof.
26. A modified bacterial organism that has altered hydrocarbon production relative to the wild-type bacterial organism.
27. A modified bacterial organism that has altered ketone production relative to a corresponding unmodified bacterial organism.
28. The modified bacterial organism of embodiment 26 or embodiment 27 wherein the organism produces one or more ketones and comprises a modified OleABCD nucleic acid region encoding the corresponding ABCD proteins.
29. The modified bacterial organism of embodiment 28 which produces substantially no OleB protein.
30. The modified bacterial organism of embodiment 28 wherein the modified OleABCD nucleic acid region encodes OleA protein and substantially no OleB protein, OleC protein, or OleD protein.
31. The modified bacterial organism of embodiment 28 wherein the modified OleABCD nucleic acid region encodes OleA protein and substantially no OleB protein, OleC protein, or OleD protein.
32. The modified bacterial organism of embodiment 26 or embodiment 27 containing substantially no genomic nucleic acid that encodes OleA, OleB, OleC, or OleD proteins and comprising nucleic acid encoding a heterologous protein having fatty acyl condensase function.
33. The modified bacterial organism of embodiment 32 further comprising regulatory elements to regulate produceion of the nucleic acid encoding the heterologous protein having fatty acyl condensase function.
34. The modified bacterial organism of embodiment 32 wherein the regulatory elements comprise promoters, silencers, enhancers, and combinations thereof.
35. The modified bacterial organism of any one of embodiments 26 through 34 which is a Shewanella bacterium.
36. The modified bacterial organism of embodiment 35 which is a Shewanella oneidensis bacterium.
37. The modified bacterial organism of embodiment 36 which is a Shewanella oneidensis strain MR-1.
38. A method of modifying a bacterial organism to produce altered hydrocarbon production relative to the wild-type bacterial organism comprising:
   removing genomic nucleic acid that encodes OleA, OleB, OleC, or OleD proteins; and
   inserting nucleic acid that encodes a heterologous protein having fatty acyl condensase function.
39. A method of controlling the synthesis of a hydrocarbon, the method comprising:
   providing a modified bacterial organism of any one of embodiments 26 through 37; and
   culturing the modified bacterial organism under conditions effective to produce one or more hydrocarbons.
40. A method of controlling the synthesis of a ketone, the method comprising:
   providing a modified bacterial organism of any one of embodiments 26 through 37; and
   culturing the modified bacterial organism under conditions effective to produce one or more ketones.
41. A method of controlling the synthesis of an energy storage molecule, the method comprising:
   providing a modified bacterial organism of any one of embodiments 26 through 37; and
   culturing the modified bacterial organism under conditions effective to produce one or more energy storage molecules.
42. The method of any one of embodiments 38 through 41 further comprising regulating the substrate composition available to the modified bacterial organism for conversion.
43. The method of embodiment 42 wherein the substrate composition comprises one or more fatty acids.
44. The method of embodiment 43 wherein the one or more fatty acids comprise one or more unsaturated fatty acids (preferably, mono-unsaturated or di-unsaturated), one or more saturated fatty acids, or combinations thereof.
45. A hydrocarbon mixture produced by the method of embodiment 39 or any one of embodiments 42 through 44 as they depend on embodiment 39.
46. A ketone mixture produced by the method of embodiment 40 or any one of embodiments 42 through 44 as they depend on embodiment 40.
47. An isolated and purified nucleic acid construct comprising nucleic acids encoding an oleA protein.
48. A vector comprising the isolated and purified nucleic acid of embodiment 47.
49. A cell comprising the vector of embodiment 48.
50. A method of extracting a mixture of ketones from a biological culture comprising:
   providing a culture comprising a modified bacterial organism of any one of embodiment 27 and embodiments 28 through 37 as dependent on embodiment 27;
   growing the culture under conditions wherein said ketones are produced in said culture;
   preparing an organic extract from said culture; and
   purifying ketones from said extract;
   thereby producing an extract containing a mixture of ketones.

### EXAMPLES

Objects and advantages of this invention are further illustrated by the following examples, but the particular materials and amounts thereof recited in these examples, as well as other conditions and details, should not be construed to unduly limit this invention.

I. Head-to-Head Hydrocarbon in Shewanella oneidensis Strain MR-1: Structure, Function and Insights Into Biosynthesis

A polyolefinic hydrocarbon was found in non-polar extracts of Shewanella oneidensis MR-1 and identified as 3,6,9,12,15,19,22,25,28-hentriacontanonaene (I) by mass spectrometry, chemical modification, and nuclear magnetic resonance spectroscopy. Compound I was shown to be the product of a head-to-head fatty acid condensation biosynthetic pathway dependent on genes denoted as ole (olefin biosynthesis). Four ole genes were present in S. oneidensis MR-1. Deletion of the entire oleABCD gene cluster led to the complete absence of non-polar extractable products. Deletion of the oleC gene alone generated a strain that lacked compound I, but produced a structurally analogous ketone. Complementation of the oleC gene eliminated formation of the ketone and restored the biosynthesis of compound I. A recombinant S. oneidensis strain containing oleA from Stenotrophomonas maltophilia strain R551-3 produced at least 17 related long-chain compounds in addition to compound I, 13 of which were identified as ketones. A potential role for OleA in head-to-head condensation was proposed. It was further proposed that long-chain polyunsaturated compounds aid in adapting cells to a rapid drop in temperature, based on three observations. In S. oneidensis wild-type cells, the cellular concentration of polyunsaturated compounds increased significantly with decreasing growth temperature. Secondly, the oleABCD deletion strain showed a significantly longer lag phase compared to the wild-type strain when shifted to a lower temperature. Lastly, compound I has been identified in a significant number of bacteria isolated from cold environments.

### I-A. Materials and Methods

Bacterial strains, culture conditions and growth. A list of Shewanella strains used in this study can be found in Table I-1. Cultures of S. oneidensis MR-1 were routinely grown in Luria-Bertani (LB) medium under ideal conditions (aerobic, 30° C.) unless stated otherwise. Cultures were grown to early stationary phase at 36° C., 22° C., 15° C. or 4° C. for experiments in which the relative amount of hydrocarbon was determined (n=6). In cold-adaption experiments (n=6), the oleABCD mutant and wild-type strains were first grown to a similar OD on LB medium overnight at 30° C. and then diluted by the same dilution factor into fresh medium at 4° C. with a beginning optical density (OD) of approximately 0.01. Aerobic growth was continued at 4° C. and optical densities were measured using a Beckman DU 7400 Spectrophotometer. For each treatment (6 flasks), three OD measurements were made and then averaged.

For maintenance of plasmids in S. oneidensis strains, 50 μg/ml of kanamycin (Km) was added to the media. For selection for recombinants (see Section: Mutagenesis), Km was added to a final concentration of 50 μg/ml while sucrose was added to a final concentration of 5% (w/v). Escherichia coli strains and their genotypes are listed in Table I-1. All E. coli strains were grown aerobically at 37° C. in LB. Where appropriate, Km was added to the growth medium at a final concentration of 50 μg/ml and diaminopimelic acid was added to a final concentration of 0.3 mM.

TABLE I-1
Strains and plasmids used in this study
*Saltikov et al. 2003. Proc. NAS. 19: 10983-10988.

| Strain or plasmid | Genotype or relative characteristic(s) | Ref |
|---|---|---|
| Strains | | |
| Shewanella oneidensis MR-1 | Wildtype | |
| Shewanella oneidensis Δole | S. oneidensis MR-1, Δole; does not produce | This |
| | hydrocarbon | study |
| Shewanella oneidensis ΔoleC | S. oneidensis MR-1, ΔoleC; does not produce | This |
| | hydrocarbon | study |
| Shewanella oneidensis ΔpfaA | S. oneidensis MR-1, ΔpfaA; does not produce | This |
| | hydrocarbon | study |
| Escherichia coli UQ950 | E. coli DH5α λ(pir) host for cloning; F- | * |
| | Δ(argF-lac)169 Φ80dlacZ58(ΔM15) | |
| | glnV44(AS) rfbD1 gyrA96(NalR) recA1 endA1 | |
| | spoT1 thi-1 hsdR17 deoR λpir+ | |
| Escherichia coli WM3064 | Donor strain for conjugation: thrB1004 pro thi | * |
| | rpsL hsdS lacZΔM15 RP4-1360 | |
| | Δ(araBAD)567 ΔdapA1341::[erm pir(wt)] | |
| Plasmid | | |
| pSMV3 | 9.5-kb vector; Km^{r}-only version of pSMV8; | * |
| | lacZ; sacB | |
| pSMV3-Δole | 2.3-kb fusion PCR fragment containing Δole | This |
| | cloned into the SpeI/SacI site of pSMV3; used | study |
| | to make the S. oneidensis Δole strain | |
| pSMV3-ΔoleC | 2.2-kb fusion PCR fragment containing ΔoleC | This |
| | cloned into the SpeI/SacI site of pSMV3; used | study |
| | to make the S. oneidensis ΔoleC strain | |
| pSMV3-ΔpfaA | 2.0-kb fusion PCR fragment containing ΔpfaA | This |
| | cloned into the SpeI/ApaI site of pSMV3; used | study |
| | to make the S. oneidensis ΔpfaA strain | |
| pBBR1MCS-2 | 5.1-kb broad-host range plasmid; lacZ; Km^{r} | ** |
| pOleC | 2.1-kb PCR fragment containing the S. oneidensis | This |
| | oleC, cloned into the SpeI/SacI site | study |
| | of pBBR1MCS-2 | |
| pPfaA | 7.6-kb PCR fragment containing the S. oneidensis | This |
| | pfaA, cloned into the ApaI/SpeI site | study |
| | of pBBR1MCS-2 | |
| pOleA-S.m. | 1.1-kb PCR fragment containing the S. maltophilia | This |
| | oleA, cloned into the SpeI/SacI site | study |
| | of pBBR1MCS-2 | |

*Saltikov et al. 2003. Proc. NAS. 19: 10983-10988.
** Kovach et al. 1995. Gene 166: 175-176.

Hydrocarbon ad ketone analysis. Hydrocarbons and ketones were analyzed by gas chromatography-mass spectrometry as previously described (Frias et al. 2009. Appl. Environ. Microbiol. 75:1774-1777). Early stationary-phase cultures, cells and media together, were extracted. The resulting evaporated residue was recovered in 1 ml methyl-t-butyl ether and applied to a 4.0 g silica gel column, eluted with 35 ml hexanes, concentrated, and subjected to molecular distillation using a Bantamware sublimation apparatus. The hydrocarbon distillate was collected between 100-115° C. (0.02 Torr); the ketone distillate between 120-130° C. (0.02 Torr). The distillates were recovered in 1 ml pentanes and subjected to GC-MS analysis using an HP6890 gas chromatograph connected to an HP5973 mass spectrometer (Hewlett Packard, Palo Alto, Calif.). GC conditions consisted of: helium gas, 1 ml/min; HP-1 ms column (100% dimethylpolysiloxane capillary 30 m by 0.25 mm by 0.25 μm); temperature ramp, 100-320° C.; 10° C./min, with a 5 min hold at 320° C. The mass spectrometer was run in electron impact mode at 70 eV and 35 μA.

The 3,6,9,12,15,19,22,25,28-hentriacontanonaene (I) produced by wild-type S. oneidensis MR-1 was purified and identified through GC-MS and NMR analyses. NMR was performed using a Varian INOVA 500 MHz NMR. Olefin hydrogenation used 5% palladium on carbon as the catalyst under hydrogen at 1-2 atm pressure. Chemical characterization: TLC (hexanes:dichloromethane, 80:20 v/v): R_{F}=0.13; (hexanes: dichloromethane, 80:20 v/v, silver nitrate): R_{F}=0.027; ¹H-NMR (500 MHz, CDCl₃): 5.28-5.45 p.p.m. (17.8H), 2.76-2.92 (14.0H), 2.14-2.22 (3.9H), 2.00-2.12 (4.8H), 0.94-1.02 (5.9H); UV/vis: λₘₐₓ 208 nm; medium resolution-MS (m/z): [M]⁺ calculated for C₃₁H₄₆: 418.7. found: 418.3.

Mutagenesis. Deletion of the oleABCD cluster and oleC from MR-1 was achieved utilizing homologous recombination between flanking regions of the target gene(s) cloned into a suicide vector (Saltikov et al. 2003. Proc. NAS. 19:10983-10988). Briefly, upstream and downstream regions of the target deletion were cloned into the suicide vector pSMV3 in a compatible E. coli cloning strain UQ950. The suicide vector was transformed into an E. coli mating strain WM3064 and then conjugated into MR-1. The initial recombination event was selected for by resistance to Km. Cells containing the integrated suicide vector were grown in the absence of selection overnight at 30° C., then plated onto LB plates containing 5% sucrose (Saltikov et al. 2003. Proc. NAS. 19:10983-10988). Cells retaining the suicide vector were unable to grow due to the activity of SacB, encoded on the vector, while cells that have undergone a second recombination event formed colonies. Colonies were then screened by PCR to determine strains containing the deletion. For creation of the oleABCD cluster-knockout strain, primers oleclusterUF, oleclusterUR, oleclusterDF, and oleclusterDR containing SpeI, BsaI, BsaI, and SacI restriction sites, respectively, were designed for the regions flanking the two ends of the oleABCD cluster (GIs: 24373309, 24373310, 24373311, and 24373312 respectively; Locus tags: SO_{—}1742, SO_{—}1743, SO_{—}1744, and SO_{—}1745 respectively). For creation of the oleC knockout strain, primers oleCUF, oleCUR, oleCDF, and oleCDR containing SpeI, BsaI, BsaI, and SacI restriction sites, respectively, were designed for the regions flanking the ends of oleC (GI: 24373311; Locus tag: SO_{—}1744). Finally, for the creation of the pfaA knockout strain, primers pfaA1F, pfaA1R, pfaA2F, and pfaA2R containing the SpeI, BamHI, BamHI, and ApaI restriction sites, respectively, were designed for the regions flanking the ends of pfaA (GI: 24373171; Locus tag: SO_{—}1602). Primer names and sequences are listed in Table I-2.

TABLE I-2
Primers used in this study.

| Primer | Sequence |
|---|---|
| oleclusterUF | TTACTAGTATCATGCCAACCCTTTTCGC |
| oleclusterUR | TTGGTCTCCATCGGATAATTGATGCC |
| oleclusterDF | TTGGTCTCTCGATAGAAGAGGGGATG |
| oleclusterDR | AAGAGCTCGCACTCGGTGTTGATACAAA |
| oleCUF | TTACTAGTTTTAACGAAGGTGCGCTAAGG |
| oleCUR | AAGGTCTCCTCGAACAGCGCATCATCCA |
| oleCDF | TTGGTCTCATCGAGCTTGATCAATCTTT |
| oleCDR | AAGAGCTCCAGCTTCAGCTTACCTAAAC |
| pfaA1F | ACTAGTGCACTCAAGTCGCAGATATTGTTCGCA |
| pfaA1R | GGATCCACCAACGATGGCAATGGGCAT |
| pfaA2F | GGATCCAGTAAGACGCTTAACCAAGCAT |
| pfaA2R | GGGCCCGGTCAATGAATCAATCAGTTGCAACAAC |
| SO1744Fcomp | ACTAGTGATTACCCATATCAAGCACTTTATGACT |
| | GAGA |
| SO1744Rcomp | GAGCTCTTGAATGCAATGGGATAATGTTTCATCCC |
| pfaAcomplementF | GGGCCCATGAGCCATACCCCTTCACAGCCT |
| pfaAcomplementR | ACTAGTTAATGCGGCATGTGCGATTGGGTTGAGTG |
| SmclusterCompF | ACTAGTCCCCCTTTTGCCTGAGCCTTGGCGC |
| SmthiolaseCompR | GAGCTCGAAGATCATCGCTGTCCGTCGCGAGC |

Mutant complementation and heterologous gene produceion: Complementation of the oleC and pfaA mutants were performed using the pBBR1MCS-2 produceion vector (Kovach et al. 1995. Gene 166:175-176) using the endogenous lac promoter (which is constitutive in MR-1 due to the absence of lad), and Shine-Dalgarno sites of the vector. Primers SO1744Fcomp and SO1744Rcomp containing SpeI and SacI restriction sites or pfaAcomplementF and pfaAcomplementR containing ApaI and SpeI restriction sites were designed for the regions flanking the ends of oleC (GI: 24373311; Locus tag: SO_{—}1744) or pfaA (GI: 24373171; Locus tag: SO_{—}1602), respectively. The Stenotrophomonas maltophilia oleA (GI: 194363945; Locus tag: Smal_{—}0167) was amplified using primers SmclusterCompF and SmthiolCompR containing the SpeI and SacI restriction sites. Resulting PCR products were ligated into the Strataclone cloning system (Agilent Technologies) followed by ligation of the product into the pBBR1MCS-2 produceion vector. Constructs were introduced into E. coli WM3064 and conjugated into the oleC deletion, pfaA deletion, or wild-type S. oneidensis MR-1 strain. Appropriately orientated inserts were verified by PCR analysis. The produceion of the cloned genes were verified by detection of product activity using GC-MS analysis.

Sequence analysis. Sequences comparisons were made using the National Center for Biotechnology Information BLAST (bl2seq) tool. Ole protein sequences from S. oneidnesis MR-1 and M. luteus were compared. GI numbers and sequences were obtained from the GenBank database.

### I-B. Results and Discussion

Long-chain hydrocarbon present in S. oneidensis cells at all growth phases. The hydrocarbon was identified in the non-polar fraction following solvent extraction from the cultures. Gas chromatography-mass spectrometry showed a single sharp peak at 20.2 min that had a parent ion at 418 mass units (FIG. I-1A). Reduction of the product with hydrogen yielded a single product with a slightly longer retention time and a parent ion of 436 mass units (FIG. I-1). The reduced product behaved identically to the C₃₁ n-alkane hentriacosane. This indicated that the biological product was a hentriacontanonaene, but the positions of the nine double bonds could not be deduced from mass spectrometry. The compound had no appreciable UV absorbance above 230 nm, suggesting that the double bonds were not in conjugation. The proton NMR was decisive (FIG. I-2) and consistent with one nearly centrosymmetric structure only; specifically, 3,6,9,12,15,19,22,25,28-hentriacontanonaene (I). The absolute stereochemistry at the double bonds remains to be determined, but is shown in the figure as all-cis because of further data on its biosynthetic origin (see below). The structure of I was consistent with it being derived from a head-to-head condensation between two fatty acyl chains to produce long-chain olefins containing a double bond between the central and an adjacent carbon atom in the chain.

Origin of the fatty acids undergoing head-to-head condensation. The structure of the hydrocarbon (I) produced by S. oneidensis MR-1 would require the condensation of two molecules of hexa-4,7,10,13-tetraenoic acid or an acyl equivalent of this; for example, the acyl-CoA derivative. This specific acyl derivative is known to be an intermediate in the biosynthesis of long chain polyunsaturated fatty acids (PUFAs) (Metz et al. 2001. Science 293: 290-293). PUFAs such as eicosapentaenoic acid are known to be produced by various Shewanella species (Bowman et al. 1997. Int. J. Syst. Bacteriol. 47:1040-1047). Moreover, PUFA biosynthetic genes from Shewanella have been identified by heterologous produceion (Jeong et al. 2006. Biotechnol. Bioprocess Eng. 11: 127-133) and in S. oneidensis strain MR-1 via genome annotation (Heidelberg et al. 2002. Nat. Biotech. 20: 1118-1123).

To confirm the involvement of the PUFA pathway genes in the biosynthesis of compound I, a pfaA (annotated as a multi-domain beta-keto acyl synthase; GI: 24373171; Locus tag: SO_{—}1602) deletion mutant was constructed. When this mutant was tested for hydrocarbon biosynthesis, neither compound I, nor any hydrocarbon product, could be detected. Hydrocarbon biosynthesis was restored by the presence of the plasmid-encoded pfaA (data not shown).

Genetic analysis of ole gene homologs. We next sought to study the genes responsible for the condensation of a PUFA intermediate leading to the formation of compound I. A cluster of genes in Shewanella oneidensis MR-1 was observed to be homologous to genes (ole) previously implicated in head-to-head hydrocarbon biosynthesis (Friedman et al. 2008. International Publication Number WO 200.8/147781; Friedman et al. 2008. International Publication Number WO 2008/113041). These were Shewanella proteins GI:24373309, GI:24373310, GI:24373311, GI:24373312 that were annotated in the GenBank database as a 3-oxoacyl-(acyl carrier protein) synthase III, an alpha/beta fold family hydrolase, a peptide hydrolase, and a 3-hydroxysteroid dehydrogenase/isomerase family protein, respectively. The first protein (GI:24373309), had 31% sequence identity to the Mlut_{—}13230 protein identified by Beller, et al to be involved in a head-to-head pathway in M. luteus (Beller et al. Appl. Environ. Microbiol. 76:1212-1223). The two proteins GI:243733310 and 24373311 from S. oneidensis MR-1 resembled the N-terminus and carboxy-terminus, respectively, of the protein Mlut_{—}13240 in M. luteus. Protein 4 (GI:24373312) showed 31% sequence identity to the Mlut_{—}13250 protein of M. luteus. The bioinformatics data suggested that S. oneidensis MR-1 proteins GI:24373309 through 24373312 were, like the M. luteus proteins, involved in a head-to-head condensation reaction. This was investigated genetically to both confirm these gene's involvement and to investigate the effect of gene alteration on product formation.

The choice of S. oneidensis strain MR-1 allowed us to use well-established gene deletion methods to test if the oleABCD genes are involved in olefin biosynthesis (FIG. I-3A). In-frame deletions of the entire ole cluster, and of oleC individually, were generated. The gene deletion was verified using PCR. A 1.7 kb band corresponding to the oleC-containing gene cluster in the wild-type became a 0.3 kb fragment in ΔoleC resulting from deletion of the 1.5 kb oleC (FIG. I-3B). The complement shows both 0.3 and 1.7 kb bands representing the deleted gene region plus the full oleC present on the pOleC plasmid. FIG. I-3C shows the gas chromatograph of the region where compound I, produced by wild-type S. oneidensis, elutes at approximately 20.2 min. The oleC mutant showed no detectable peak in this region. The complemented strain showed a restoration of the 20.2 min peak. The identity of the compound eluting at 20.2 min was confirmed by mass spectrometry. GC experiments were performed in triplicate. Similarly, the oleABCD deletion strain did not produce compound I (FIG. I-1S).

Formation of ketones and implications for the function of OleA. The S. oneidensis MR-1 oleC deletion mutant did not produce a hydrocarbon, but it made another compound that was purified from a different distillation fraction than the hydrocarbon. The mass spectrum of the compound, III, had a parent ion of m/z 434. These data were consistent with a symmetrical molecule with 8 double bonds and having the carbonyl functionality at the center of the hydrocarbon chain. Compound III was hydrogenated to produce a molecule with m/z 450, and showed an ion fragment of m/z 239. This confirmed the structure of III to be 3,6,9,12,19,22,25,28-hentriacontaoctaene-16-one. Compound III was not found in the S. oneidensis MR-1 oleABCD mutant.

Ketone products were also observed in an additional experiment involving heterologous oleA gene produceion into S. oneidensis MR-1. The oleA gene homolog from S. maltophilia strain R551-3 was cloned into S. oneidensis strain MR-1. The heterologous strain grew normally but produced a much wider range of non-polar extractable products (FIG. I-4). The endogenous compound I was present and readily identified by GC retention time and mass spectrum and is shown in FIG. I-4 with an asterisk and the chemical formula, C₃₁H₄₆. The recombinant Shewanella strain produced at least 17 additional long-chain compounds, of which 13 were monoketones (FIG. I-4). The chemical formulas are shown, indicating the degree of unsaturation of the hydrocarbon chains. All of the compounds are significantly more saturated than the endogenous C₃₁H₄₆ hydrocarbon, suggesting that the Stenotrophomonas OleA protein, unlike the Shewanella OleA protein, condenses fatty acids not derived from the polyunsaturated fatty acid pathway. The ketones were identified from their characteristic mass spectra; both the parent ions and ion fragments were consistent with these assignments. Moreover, the observation of a single major carbonyl ion, or two such ions of similar molecular weight, is consistent with the carbonyl functional group being present at the median carbon for odd numbered chain lengths. This observation is consistent with these products arising from a head-to-head fatty acid condensation mechanism.

The data shown in FIG. I-4 was striking because the native Shewanella only made a single endogenous C₃₁H₄₆ hydrocarbon, compound I. By contrast, S. maltophilia is known to produce a large number of different hydrocarbons with chain lengths of C₂₆-C₃₀ (Suen et al. 1988. J. Ind. Microbiol. 2:337-48) and the S. maltophilia oleA gene alone directed the formation of a much wider range of products in Shewanella. The observation here of diverse hydrocarbons and ketones has implications for the production of molecules for fuel or specialty chemical applications via the heterologous produceion of different oleA genes in Shewanella.

Ketone formation could potentially result from the OleA protein alone and this would be consistent with the data presented here. OleA is in the thiolase superfamily that catalyzes both decarboxylative and non-decarboxylative acyl group condensation reactions (Haapalainen et al. 2005. Trends Biochem. Sci. 31:64-71; Heath et al. 2002. Nat. Prod. Rep. 19:581-96). A non-decarboxylative thiolytic condensation would produce an intermediate that could give rise to ketones (FIG. I-5). FIG. I-5 shows the structure of the natively-produced polyolefin, compound I. Hydrocarbons and ketones could both be derived from an intermediate generated by OleA and that is consistent with reactions catalyzed by thiolase superfamily members, of which OleA is a member. Thioester cleavage could occur by the action of: (a) OleA, (b) a thioesterase, or (c) spontaneous hydrolysis (Fredslund et al. 2006. J. Mol. Biol. 361:115-127) to generate a β-keto acid (compound II in FIG. I-5C). β-Keto acids are known to be unstable and decarboxylate spontaneously (Pedersen et al. 1929. J. Am. Chem. Soc. 51:2098-2107). Spontaneous decarboxylation of β-keto acids in biological systems is well-known and underlies the production of ketone bodies in mammalian liver (Hird et al. 1962. Biochem. J. 84:212-216). In the case of the S. oneidensis MR-1 oleC mutant, intermediate II would be generated and decarboxylate to generate compound III, the observed ketone. When the OleA from Stenotrophomonas was produced in Shewanella, a narrower specificity for the Shewanella enzymes could lead to the build up of different intermediates that undergo hydrolysis and decarboxylation to yield the ketones. An alternative mechanism for the OleA-catalyzed condensation reaction is proposed in the literature (Beller et al. Appl. Environ. Microbiol. 76:1212-1223).

Potential role of ole gene product(s) in cold adaption. A hydrocarbon that appears to be identical to compound I was previously identified in a significant number of Antarctic bacterial isolates (Nichols et al. 1995. FEMS Microbiol. Lett. 125:281-286). The hypothesis that long-chain olefins might contribute to cold adaption was tested directly with S. oneidensis strain MR-1 wild-type, which grows within the temperature range of 4-37° C. (optimal growth at 30° C.). The first observation supporting the hypothesis in this study was that decreasing the growth temperature led to significant increases in the amount of compound I and compound III present in cells (FIG. I-6A).

In other experiments, wild-type and olefin-deficient strains were grown at 30° C. and then inoculated into medium at 4° C. (FIG. I-6B). Although there was not much difference in the growth rate during exponential phase, the olefin-deficient oleABCD mutant strain showed a significantly longer lag phase prior to exponential growth (FIG. I-6B). When the oleABCD mutant was pre-grown at 4° C., this lag in growth following transfer was not observed. These data suggested at least one role for long-chain olefins in facilitating growth following a shift to colder temperatures. We expect that the polyolefin would increase membrane fluidity, a beneficial property following a decrease in temperature.

Structurally analogous long chain alkadienes and alkatrienes are prominent in the lipids of marine photosynthetic eukaryotes such as Isochrysis galbana that grow at cold oceanic temperatures (Rieley et al. 1998. Lipids 33:617-625). They are also present, along with long-chain alkenones, in the lipid fractions of Emiliania huxleyi (Rieley et al. 1998. Lipids 33:617-625), a photosynthetic eukaryote which is so common that oceanic algal blooms are observable by satellite photographs (Brown et al. 1994. J. Geophys. Res. 99(C4): 7467-7482). The mechanism of hydrocarbon formation in these eukaryotes remains open, but our findings here, coupled with ongoing genome sequencing of these organisms, may help provide insight. It is interesting that the amount and degree of unsaturation of the long-chain hydrocarbons and alkenones increase with decreasing temperature (Prahl et al. 1987. Nature 330:367-369). This suggests that long-chain hydrocarbons and ketones could be involved in cold adaption in both bacteria and eukaryotes.

### II. Widespread Head-to-Head Hydrocarbon Biosynthesis in Bacteria and the Role of OleA

Previous studies identified the oleABCD genes involved in head-to-head olefinic hydrocarbon biosynthethesis. The present study more fully defined the OleABCD protein families within the thiolase, α/β-hydrolase, AMP-dependent ligase/synthase, and short chain dehydrogenase superfamilies, respectively. Only 0.1-1% of each superfamily represent likely Ole proteins. Sequence analysis based on structural alignments and gene context was used to identify highly likely ole genes. Selected microorganisms from the Phyla Verucomicrobia, Planctomyces, and Chloroflexi, Proteobacteria, and Actinobacteria were tested experimentally and shown to produce long-chain olefinic hydrocarbons. However, different species from the same genera sometimes lack the ole genes and fail to produce olefinic hydrocarbons. Overall, only 1.9% of 3558 genomes analyzed showed clear evidence for containing ole genes. The type of olefins produced by different bacteria differed greatly with respect to the number of carbon-carbon double bonds. The greatest number of organisms surveyed biosynthesized a single long-chain olefin, 3,6,9,12,15,19,22,25,28-hentriacontanonaene, that contained nine double bonds. Xanthomonas campestris produced the greatest number of distinct olefin products, fifteen compounds ranging in length from C₂₈ to C₃₁ and containing one to three double bonds. The type of long-chain product formed was shown to be dependent on the oleA gene in experiments with Shewanella oneidensis MR-1 ole gene deletion mutants containing native or heterologous oleA genes produced in trans. A strain deleted in oleABCD and containing oleA in trans produced only ketones. Based on these observations, it was proposed that OleA catalyzes a non-decarboxylative thiolytic condensation of fatty acyl chains to generate a β-ketoacyl intermediate that can decarboxylate spontaneously to generate ketones.

### II-A. Materials and Methods

Strains and culture conditions. Wild-type and recombinant bacteria used in this study are listed in Table II-1. All organisms, including recombinant strains, were grown aerobically in 50 ml culture flasks on a rotary shaker at 225 rpm except for Geobacter strains which were grown in 100 ml anaerobic culture flask flushed for 30 minutes with a nitrogen/carbon dioxide gas mix prior to culture inoculation (Rollefson et al. 2009. J. Bacteriol. 191:4207-4217). All organisms were grown at 30° C. (Bauld et al. 1976. J. Gen. Microbiol. 97:45-55; Burnes et al. 2000. Appl. Environ. Microbiol. 66:5201-5205; Kinoshita et al. 1988. J. Ferment. Technol. 66:145-152; Kovacs et al. 1999. Int. J. Syst. Bacteriol. 49:167-173; Rollefson et al. 2009. J. Bacteriol. 191:4207-4217) except for Shewanella amazonensis (35° C.) (71), S. frigidimarina (22° C.) (Venkateswaran et al. 1999. Int. J. Syst. Bacteriol. 49:705-724), Opitutaceae bacterium TAV2 (22° C.) (Stevenson et al. 2004. Appl. Environ. Microbiol. 70:4748-4755), Brevibacterium fuscum (22° C.) (Kinoshita et al. 1988. J. Ferment. Technol. 66:145-152), and Colwellia psychrerythraea (4° C.) (Yumoto et al. 1998. Int. J. Syst. Bacteriol. 48:1357-1362), Chloroflexus aurantiacus (55° C.) (Pierson et al. 1974. Arch. Microbiol. 100:5-24), and all Escherichia coli strains (37° C.) (Saltikov et al. 2003. Proc. Natl. Acad. Sci., USA 19:10983-10988), and allowed to achieve stationary phase prior to hydrocarbon extraction and analysis. All organisms were grown in Luria broth (DIFCO) (Kinoshita et al. 1988. J. Ferment. Technol. 66:145-152; Kovacs et al. 1999. Int. J. Syst. Bacteriol. 49:167-173; Venkateswaran et al. 1998. Int. J. Syst. Bacteriol. 48:965-972; Venkateswaran et al. 1999. Int. J. Syst. Bacteriol. 49:705-724) except for S. frigidimarina (Bowman et al. 1997. Int. J. Syst. Bacteriol. 47:1040-1047), C. psychrerythraea (Yumoto et al. 1998. Int. J. Syst. Bacteriol. 48:1357-1362), and P. maris (Marine broth, DIFCO) (Ward-Rainey et al. 1997. J. Bacteriol. 179:6360-6366), Geobacter species (Geobacter medium, DSMZ) (Rollefson et al. 2009. J. Bacteriol. 191:4207-4217), C. aurantiacus (Chloroflexus media, DSMZ) (Pierson et al. 1974. Arch. Microbiol. 100:5-24), Opitutaceae bacterium TAV2 (R2A medium, DIFCO) (Schmidt, personal communication), and X. campestris (Nutrient Broth, DIFCO) (Burnes et al. 2000. Appl. Environ. Microbiol. 66:5201-5205).

TABLE II-1
Organisms, plasmids, and primers used in this study

| | | Source |
|---|---|---|
| Organism, plasmid, or primer | Genotype or relevant characteristic(s) | or Ref. |
| Genetically modified organisms | | |
| S. oneidensis ΔoleA | S. oneidensis MR-1, ΔoleA; hydrocarbon minus | This study |
| S. oneidensis Δole | S. oneidensis MR-1, Δole; hydrocarbon minus | 1 |
| E. coli UQ950 | E. coli DH5α λ(pir) host for cloning; F-Δ | 2 |
| | (argF-lac) 169 Φ80dlacZ58(ΔM15) glnV44(AS) | |
| | rfbD1 gyrA96 (NalR) recA1 endA1 spoT1 thi-1 | |
| | hsdR17 deoR λpir+ | |
| E. coli WM3064 | Donor strain for conjugation: thrB1004 pro | 2 |
| | thi rpsL hsdS lacZΔM15 RP4-1360 Δ(araBAD) | |
| | 567ΔdapA1341:: [erm pir(wt)] | |
| Plasmids | | |
| pSMV3 | 9.5-kb vector; Km^{r} version of pSMV8; lacZ; | 3 |
| | sacB | |
| pSMV3-ΔoleA | 0.9-kb fusion PCR fragment containing ΔoleA | This study |
| | cloned into the SpeI/BamHI site of pSMV3; | |
| | used to make the S. oneidensis ΔoleA strain | |
| pBBR1MCS-2 | 5.1-kb broad-host range plasmid; lacZ; Km^{r} | 4 |
| pOleA-S.m. | 1.1-kb PCR fragment containing the | 1 |
| | S.maltophilia olnA, cloned into the | |
| | SpeI/SacI site of pBBR1MCS-2 | |
| pOleA | 1.9-kb PCR fragment containing the | This study |
| | S. oneidensis oleA, cloned into the SpeI/ | |
| | SacI site of pBBR1MCS-2 | |
| Primers | | |
| oleASoF1 | ACTAGTTACATGTGCGTTTATTGCAACTGGCC | |
| oleASoR1 | CCAGAGATATAGAGGCGCGAGGCGAGATTC | |
| oleASoF2 | GGTCTCATGGCACACGATCAAGGCTTTTTAC | |
| oleASoR2 | GGATCCCCAACAAATCAGTGTCGGCACC | |
| SooleACompF | ACTAGTTACATGTGCGTTTATTGCAACTGGCC | |
| SooleACompR | GAGCTCGTTAAAGCATCGGCTAAGGCAGATAACAA | |

Wild-type organisms
Shewanella oneidensis MR-1 (5,6), Shewanella putrefaciens CN-32 (7), Shewanella baltica OS185 (8), Shewanella frigidimarina NCIMB 400 (9), Shewanella amazonensis SB2B (10), Shewanella denitrificans OS217 (11), Colwellia psychretythraea 34H (12), Geobacter bemidjiensis Bem (13), Geobacter sulfurreducens PCA (14), Opitutaceae bacterium TAV2 (15), lanctomyces marls DSM 8797 (16), Chloroflexus aurantiacus J-10-fl (17), Kocuria rhizophila DC2201 (18), Brevibacterium fuscum ATCC 15993 (19), Xanthomonas campestris (20), Vibrio furnissii MI (21), E. coli K12 (lab supply)
REFS: 1. Sukovich et al. 2010. Appl. Environ. Microbiol.; 2. Saltikov, C. W. and D. K. Newman. 2003. Genetic identification of a respiratory arsenate reductase. Proc. Natl. Acad. Sci., USA 19: 10983-10988; 3. Baron et al. 2009. J. Biol. Chem. 284: 28865-28873; 4. Kovach et al. 1995. Gene 166: 175-176; 5. Stackebrandt et al. 1999. Int. J. Syst. Bacteriol. 49: 705-724; 6. Venkateswaran et al. 1999. Int. J. Syst. Bacteriol. 49: 705-724; 7. Jorgensen et al. 1989. Int. J. Food Microbiol. 9: 51; 8. Ziemke et al. Int. J. Syst. Bacteriol. 8: 179-186; 9. Bowman et al. 1997. Int. J. Syst. Bacteriol. 47: 1040-1047; 10. Venkateswaran et al. 1998. Int. J. Syst. Bacteriol. 48: 965-972; 11. Brettar et al. 2002. Int. J. Syst. Evol. Microbiol. 52: 2211-2217; 12. Deming et al. 1988. Appl. Microbiol. 10: 152-160; 13. Nevin et al. 2005. Int. J. Syst. Evol. Microbiol. 55: 1667-1674; 14. Caccavo et al. 1994. Appl. Environ. Microbiol. 60: 3752-3759; 15. Stevenson et al. 2004. Appl. Environ. Microbiol. 70: 4748-4755; 16. Bauld et al. 1976. J. Gen. Microbiol. 97: 45-55; 17. Pierson et al. 1974. Arch. Microbiol. 100: 5-24; 18. Kovacs et al. 1999. Int. J. Syst. Bacteriol. 49: 167-173; 19. Saito et al. 1964. Agr. Biol. Chem. 28: 48-55; 20. Vauterin et al. 1995. Int. J. Syst. Bacteriol. 45: 472-489; 21. Park et al. 2001. Appl. Microbiol. Biotechnol. 56: 448-452. 22. Beller et al. 2010, Appl.Environ.Microbiol. 76: 1212-1223. 23. Davis et al. 1987. J. iol. Chem. 262: 82-89. 24. Gavalda et al. 2009. J. Biol. Chem. 284: 19255-19264. 25. Davies et al. 2000. Structure. 8: 185-195. 26. Clinkenbeard et al. 1975. J. Biol. Chem. 250: 3124-3135.

Hydrocarbon and ketone extraction, chromatography and characterization. Early stationary-phase cultures were extracted as previously described (Wackett et al. 2007. Appl. Environ. Microbiol. 73:7192-7198). Briefly, both cells and media from a 50 ml bacterial cultures that had reached stationary phase were extracted using a mixture of spectrophotometric-grade methanol (Sigma-Aldrich), HPLC-grade chloroform (Sigma-Aldrich), and distilled water in a 1:1:0.8 ratio. The resulting non-polar phase was collected and dried under vacuum. Evaporated residue was recovered in 1 ml MTBE and applied to a 4.0 g silica gel column (Sigma-Aldrich), eluted with 35 ml HPLC-grade hexanes (Fischer Scientific), followed by 35 ml of MTBE and 25 ml of HPLC-grade ethyl acetate (Sigma). Each solvent fraction was concentrated, and subjected to GC-MS analysis using an HP6890 gas chromatograph connected to an HP5973 mass spectrometer (Hewlett Packard, Palo Alto, Calif.). GC conditions consisted of: helium gas, 1 ml/min; HP-1 ms column (100% dimethylpolysiloxane capillary, 30 m by 0.25 mm by 0.25 μm); temperature ramp, 100-320° C.; 10° C./min, with a 5 min hold at 320° C. The mass spectrometer was run in electron impact mode at 70 eV and 35 μA. Alkene and ketone products were identified from the parent ions and corresponding fragmentation patterns. Major compounds were further analyzed by hydrogenation over palladium on carbon (Sigma-Aldrich) and observing the corresponding increase in mass to confirm the number of double bonds present.

Gene deletion and oleA gene complementation. All deletion strains, plasmids, and primers used are listed in Table II-1. Gene deletions were made using homologous recombination between flanking regions of oleA cloned into a suicide vector, pSMV3 (Saltikov et al. 2003. Proc. Natl. Acad. Sci., USA 19:10983-10988). Briefly, using oleASoF1, oleASoR1, oleASoF2, and oleASoR1, the upstream and downstream regions surrounding the gene were cloned using the restriction sites SpeI and BamHI into the suicide vector in a compatible E. coli cloning strain (UQ950) (Saltikov et al. 2003. Proc. Natl. Acad. Sci., USA 19:10983-10988). This plasmid was transformed into an E. coli mating strain (WM3064) (Saltikov et al. 2003. Proc. Natl. Acad. Sci. USA 19:10983-10988) then conjugated into MR-1. While E. coli were commonly grown at 37° C., when S. oneidensis were present cells were incubated at 30° C. The initial recombination event was selected for by resistance to Kanamycin. Cells containing the integrated suicide vector grew in the absence of selection overnight at 30° C., and then were plated onto LB plates containing 5% sucrose (Saltikov et al. 2003. Proc. Natl. Acad. Sci., USA 19:10983-10988). Cells retaining the suicide vector were unable to grow due to the activity of SacB, encoded on the vector, while cells that underwent a second recombination event form colonies. Colonies were then screened by PCR to determine strains containing the deletion. The oleABCD gene cluster deletion of S. oneidensis MR-1 was created as described above.

Complementation of the S. oneidensis oleA mutant was performed using the pBBR1MCS-2 produceion vector (Kovach et al. 1995. Gene 166:175-176) using the endogenous lac promoter (which is constitutive in MR-1 due to the absence of lacI). Primers oleASoFcomp and oleASoRcomp containing SacI and SpeI restriction sites were designed for the regions flanking the ends of oleA. Resulting PCR products were ligated into the strataclone cloning system (Agilent Technologies), followed by digestion and ligation of the product into the pBBR1MCS-2 produceion vector. The Stenotrophomonas maltophilia oleA gene was introduced into pBBR1MCS-2 as described above. Constructs were introduced into E. coli WM3064 prior to conjugation with the oleA deletion, the ole cluster deletion, or wild-type MR-1 strains. All constructs were verified through PCR and sequencing analysis. Following conjugation, all constructs were maintained using kanamycin.

Identification of oleABCD containing organisms. The oleABCD genes in S. oneidensis MR-1 were used to find homologous gene clusters in the GenBank non-redundant database using the BLAST algorithm (Altschul et al. 1990. J. Mol. Biol. 215: 403-410). Subsequently, the OleA homologs in Stenotrophomonas maltophilia strain R551-3 (gi 194346749), Arthrobacter aurescens TC1 (gi119962129) and Micrococcus luteus NCTC 2665 (gi 239917824) were used as additional queries to the database. Other homologous thiolases were identified. The genome context of each of these thiolases was investigated and allowed for the assembly of a set of organisms with either a four or three gene cluster encoding OleA, B, C, and D protein domains. A lack of clustering did not preclude the existence of the pathway in an organism. Therefore, those organisms that lacked clustered genes were searched for oleBCD genes in other locations of their genome. Organisms with clustering of at least two identifiable ole homologs and had all four genes located in their genome were included as potential hydrocarbon producers and investigated experimentally.

Superfamily sequence identification and alignments. The PSI-BLAST algorithm with default conditions was used with S. oneidensis MR-1 or A. aurescens TC1 Ole protein sequences as queries. Thousands of homologous sequences were found. The sequence and catalytic diversity within each superfamily is sufficiently broad that standard sequence alignment tools did not align amino acids residues that are known to comprise the active sites in proteins for which X-ray structures are available (Conti et al. 1996. Structure. 4:287-98; Gulick et al. 2004. Biochemistry. 43:8670-9; Haapalainen et al. 2005. Trends Biochem. Sci. 31:64-71; Heath et al. 2002. Nat. Prod. Rep. 19:581-96; Jiang et al. 2008. Mol. Phylogenet. Evol. 49:691-701; Jörnvall et al. 1995. Biochemistry. 34:6003-6013; Nardini et al. 1999. Curr. Opin. Struc. Biol. 9:732-737; Qian et al. 2007. Biotechnol. J. 2:192-200; Steussy et al. 2006. Biochemistry. 45:14407-14; Thoden et al. 1997. Biochemistry. 36:10685-10695; Verschueren et al. 1993. Nature. 363:693-8; Wu et al. 2008. Biochemistry. 47:8026-39). Thus, to properly align Ole protein sequences with other proteins in their respective superfamilies, it was necessary to generate structure-based alignments. For each OleABCD alignment, 6-10 homologous proteins that had previously described high-resolution X-ray structures were structurally superposed, using the Match command in Chimera (Meng et al. 2006. BMC Bioinformatics. 7:339-349).

Conserved residues within each superfamily of homologs were derived from the literature (Conti et al. 1996. Structure. 4:287-98; Gulick et al. 2004. Biochemistry. 43:8670-9; Haapalainen et al. 2005. Trends Biochem. Sci. 31:64-71; Heath et al. 2002. Nat. Prod. Rep. 19:581-96; Jiang et al. 2008. Mol. Phylogenet. Evol. 49:691-701; Jörnvall et al. 1995. Biochemistry. 34:6003-6013; Nardini et al. 1999. Curr. Opin. Struc. Biol. 9:732-737; Qian et al. 2007. Biotechnol. J. 2:192-200; Steussy et al. 2006. Biochemistry. 45:14407-14; Thoden et al. 1997. Biochemistry. 36:10685-10695; Verschueren et al. 1993. Nature. 363:693-8; Wu et al. 2008. Biochemistry. 47:8026-39) and their locations plotted onto the protein backbone to confirm alignments. Sequence alignments based on the structure alignments were exported. Sequence alignments of each of the OleABCD families were made with 41-55 sequences, using clustalw (Chenna et al. 2003. Nucleic Acids Res. 31:497-500). In the case of the OleA alignments, 14 OleA homologs with genes that did not cluster with oleBCD genes were also included for sequence comparison purposes. A profile-profile alignment between the structural superfamily alignments and the family sequence alignments was produced, using clustalw (Chenna et al. 2003. Nucleic Acids Res. 31:497-500). These superfamily-ole sequence alignments were viewed in chimera with the overlaid superfamily crystal structures linked to the alignments so that the position of residues in the alignment could be viewed (Meng et al. 2006. BMC Bioinformatics. 7:339-349). For OleBC fusion proteins, the individual domains were used in sample alignments in the appropriate families.

Analysis of protein superfamilies. The Superfamily database (26) was searched with each of the S. oneidensis MR-1 Ole protein sequences. The superfamilies identified by these searches confirmed assignments made independently as described above. The number of distinct proteins in each superfamily was kindly provided by the Superfamily database (personal communication). The relevant superfamily categories in the Superfamily database are: thiolase-like, α/β-hydrolases, acetyl-CoA synthetase-like, and NAD(P) Rossman-fold domains. It should be noted that the NAD(P) Rossman-fold domains superfamily, as listed in the Superfamily database, consists of a number of families in which the proteins share the ability to bind NAD(P), and contains a total of 136,722 proteins as of Feb. 1, 2010. These proteins have a second domain involved in substrate binding and which confer the catalytic residues. These differentiations are made in the Superfamily database at what are denoted as the family level. The OleD proteins belong to the tyrosine-dependent-oxidoreductase domain family. This set was used for our analysis and was equivalent to the set given superfamily status by Jornvall et al. and described as the short-chain dehydrogenase/reductase superfamily (Jörnvall et al. 1995. Biochemistry. 34:6003-6013).

Network clustering of OleABCD proteins. Network clustering of each of the OleABCD proteins was done using previously described procedures (Morris et al. 2007. Bioinformatics. 23:2345-2347; Seffernick et al. 2009. J. Biotech. 143:7-26). This method was used to make an all-by-all blastp library for each of the OleABCD proteins using sequences from 15 organisms. The sequences used were: 1—S. oneidensis MR-1, OleA gi24373309, OleB gi24373310, OleC gi24373311, OleD gi24373312; 2—Shewanella amazonensis SB2B, OleA gi119774319, OleB gi119774320, OleC gi119774321, OleD gi119774322; 3—Shewanella baltica OS185, OleA gi153000075, OleB gi153000076, OleC gi153000077, OleD gi153000078; 4—Shewanella denitrificans OS217, OleA gi91792727, OleB gi91792728, OleC gi91792728, OleD gi91792730; 5—Shewanella frigidimarina NCIMB 400, OleA gi114562543, OleB gi114562544, OleC gi114562545, OleD gi114562546; 6—Shewanella putrefaciens CN-32, OleA gi146292545, OleB gi146292546, OleC gi146292547, OleD gi146292548; 7—Colwellia psychrerythraea 34H, OleA gi71279747, OleB gi71279056, OleC gi71281286, OleD gi71280771; 8—Geobacter bemidjiensis Bem, OleA gi197118484, OleB gi197118483, OleC gi197118482, OleD gi197118481; 9—Planctomyces maris DSM 8797, OleA gi149174448, OleB gi149178001, OleC gi149178707, OleD gi149178706; 10—Opitutaceae bacterium TAV2, OleA gi225164858, OleB gi225164858, OleC gi225155590, OleD not cluster; 11—Stenotrophomonas maltophilia R551-3, OleA gi194363945, OleB gi194363946, OleC gi194363948, OleD gi194363949; 12—Xanthomonas campestris pv. campestris str. B 100, OleA gi188989629, OleB gi188989631, OleC gi188989633, OleD gi188989637; 13—Chloroflexus aurantiacus J-10-fl, OleA gi163849058, OleB gi163849062, OleC gi163849060, OleD gi163849059; 14—Arthrobacter aurescens TC1, OleA gi119962129, OleB gi119960515 (residues 1-310), OleC gi119960515 (residues 389-921), OleD gi119962242; 15—Arthrobacter chlorophenolicus A6, OleA gi220911225, OleB domain gi220911226 (residues 1-296), OleC gi220911226 (residues 370-927), OleD gi220911227; 16—Kocuria rhizophila DC2201, OleA gi184200698, OleB gi184200697 (residues 1-312, OleC gi184200697 (residues 392-909), OleD gi184200696; and 17—Micrococcus luteus NCTC 2665, OleA gi239917824, OleB gi239917825 (residues 1-330), OleC gi239917825 (residues 439-978), OleD gi239917826. From these sequences, a network diagram was created. The nodes represent protein sequences and the edges represent a blast linkage that connects the two proteins. A shorter edge represents a lower e-score (greater relatedness). Expectation values from e⁻² to e⁻²⁰⁰ were analyzed for connectivity and divergence of OleA, B, C, and D protein sequence clusters, respectively.

### II-B. Results and Discussion

Ole protein superfamily analysis. Thousands of homologous sequences were identified for each of the OleA, OleB, OleC, and OleD sequences from S. oneidensis MR-1 (Table II-2). OleA is homologous to members of the thiolase superfamily, also known as the condensing enzyme superfamily. The sequence relatedness between different OleA proteins and FabH, a thiolase superfamily member, has been noted previously even though sequence identities of OleA to other superfamily members are generally low, in the range of 20-30% (Beller et al. Appl. Environ. Micro. 76: 1212-1223; Friedman et al. International Patent Application No. WO 2008/147781; Friedman et al. International Patent Application No. WO 2008/113041). OleB is a member of the α/β hydrolase superfamily. OleC is a member of the AMP-dependent ligase/synthase superfamily, also known as the acetyl-CoA synthetase-like superfamily. OleD is a member of the short chain dehydrogenase/reductase superfamily.

TABLE II-2
Ole proteins superfamilies, homolog characteristics, number of Ole proteins and homologs

| Proteins | Superfamily | | # of | # of Ole |
|---|---|---|---|---|
| in this | name | Enzymatic activities and biological | homologs in | proteins |
| study | (alternative names) | functions in the superfamily | superfamily | identified |
| OleA | Thiolase | Acyl-ACP synthase, thiolase (degradative), | 13,586 | 69 |
| | (condensing | thiolase (biosynthetic), 3-hydroxyl-3- | | |
| | enzymes) | methylglutaryl-CoA synthase, fatty acid | | |
| | | elongase, stage V sporulation protein, 6- | | |
| | | methylsalicylate synthase, Rhizobium | | |
| | | nodulation protein NodE, chalcone synthase, | | |
| | | stilbene synthase, naringenin synthase, β- | | |
| | | ketosynthase domains of polyketide synthase | | |
| OleB | α/β-Hydrolase | Esterase, haloalkane dehalogenase, protease, | 67,923 | 69 |
| | | lipase, haloperoxidase, lyase, epoxide | | |
| | | hydrolase, enoyl CoA hydratase/isomerase, | | |
| | | MhpC C—C hydrolase (carbon-carbon bond | | |
| | | cleavage) | | |
| OleC | AMP-dependent | Firefly luciferase, nonribosomal peptide | 19,660 | 69 |
| | ligase/synthase | synthase, acyl-CoA synthase (AMP forming), | | |
| | (LuxE; acyl- | 4-chlorobenzoate:CoA ligase, acetyl-CoA | | |
| | adenylate/thioester | synthetase, o-succinylbenzoic acid-CoA ligase, | | |
| | forming, Acetyl- | fatty acyl ligase, acetyl-CoA synthetase, 2-acyl- | | |
| | CoA synthetase- | glycerophospho-ethanolamine acyl transferase, | | |
| | like) | enterobactin synthase, amino acid adenylation | | |
| | | domain, dicarboxylate-CoA ligase, | | |
| | | crotonobetaine/carnitine-CoA ligase | | |
| OleD | Short-chain | Nucleoside-diphosphate sugar epimerase/ | 25,454 | 69 |
| | dehydrogenase/ | dehydratase/reductase, aromatic diol | | |
| | Reductase | dehydrogenase, steroid dehydrogenase/ | | |
| | | isomerase, sugar dehydrogenase, acetoacetyl- | | |
| | | CoA reductase, 3-oxoacyl-ACP reductase, | | |
| | | alcohol dehydrogenase, carbonyl reductase, 4- | | |
| | | α-carboxysterol-C3-dehydrogenase/C4- | | |
| | | decarboxylase, flavonol reductase, cinnamoyl | | |
| | | CoA reductase, NAD(P) dependent cholesterol | | |
| | | dehydrogenase, | | |

FIG. II-1 shows conserved regions of a structure-based multiple sequence alignment for each of the Ole A, B, C, and D proteins with three of their respective superfamily members. FIG. II-1 focuses on regions containing catalytically important residues that are highly conserved amongst the homologous proteins. A more detailed set of alignments is available in FIG. II-1S. The superfamily members shown in FIG. II-1 were selected to represent proteins serving quite different biological functions. So while OleABCD are clearly seen to contain critical catalytic residues of each respective superfamily, a precise prediction of the biochemical reaction catalyzed is difficult due to the enormous functional diversity found within each Ole protein's superfamily.

The superfamilies to which Ole proteins belong each consist of between 10⁴ and 10⁵ curated protein members that have been identified for inclusion in the Superfamily database (Table II-2). The present study suggested that only 0.1%-1% of the proteins in each superfamily represent Ole proteins that participate in head-to-head hydrocarbon biosynthesis. The identification of these Ole proteins in the sequenced genomes of microorganism is discussed below.

Protein relatedness and gene organization used to identify ole genes out of thousands of homologs. Only a limited number of bacteria to date have been found to produce long-chain olefinic hydrocarbons. For example, amongst ten Arthrobacter strains tested, six produced long-chain olefinic hydrocarbons and four did not (Frias et al. 2009. Appl. Environ. Microbiol. 75:1774-1777). Of three closely related Arthrobacter strains for which genome sequences were available, two (A. aurescens TC1 and A. chlorophenolicus A6) were shown to produce hydrocarbons and one (Arthrobacter sp. FB24) was devoid of long-chain olefinic hydrocarbons. The FB24 strain that did not produce hydrocarbons contained ole gene homologs but the percent identity was much lower, and the genes were distributed within the genome differently. By examining such divergences, a strategy for identifying highly likely ole genes was developed. In this study, the Ole protein sequences and gene organization from Shewanella oneidensis MR-1 and Arthrobacter aurescens TC1 were used as models to query genome sequence sets. OleA sequence homologs were first identified and then sequence relatedness was determined by pairwise and multiple alignments. When putative OleA proteins were identified, homologs to oleBCD genes were sought in the same genomes and examined for their relative locations. The example below is illustrative.

A putative oleA gene region was identified in Geobacter bemidjiensis Bem that, after translation, showed 58% amino acid sequence identity to the OleA protein in S. oneidensis MR-1. Directly downstream from the G. bemidjiensis Bem oleA gene, oleBCD gene homologs were present in a configuration that mirrored that of S. oneidensis MR-1 (FIG. II-2). An OleA homolog was also identified in Geobacter sulfurreducans PCA. It showed significantly lower amino acid sequence identity, 28%, to the OleA from S. oneidensis MR-1. It lacked flanking ole gene neighbors. Closer examination of the two genomes revealed that the OleA homolog in G. sulfurreducans PCA was encoded by a gene region that matched a gene region with identical synteny in G. bemidjiensis Bem. This same gene region was also identified in S. oneidensis MR-1. From this analysis, it was concluded that the OleA homolog in G. sulfurreducans PCA was not involved in a head-to-head condensation reaction and it was suggested that this organism was genetically incapable of making head-to-head olefins Cells of G. sulfurreducans PCA were tested experimentally for the presence of long-chain olefinic hydrocarbons. Hydrocarbons were absent under identical growth condition in which they were present in G. bemidjiensis Bem (see later section on hydrocarbon identification).

A collection of 3558 genomes were examined using methods as described, leading to the identification of several different ole gene arrangements (FIG. II-3; Identifiers for each of the genes are listed in Supplemental Table II-1 S below). One major distinction in ole gene organization had been recognized previously (Friedman et al. International Patent Application No. WO 2008/147781; Friedman et al. International Patent Application No. WO 2008/113041); a significant number of organisms contained either three or four separate ole genes. Of those characterized in this study, the largest set contained four contiguous oleABCD genes. However, some bacteria of the class Actinobacteria contained three ole genes, with the oleB and oleC gene regions fused into one gene (FIG. II-3A&B). Sixty-one organisms had either the four or three gene cluster readily identifiable (FIG. II-1A-D). Genomes that had a clear clustering of homologs of at least two of these genes were included as potential clusters. At least one sample organism from each of the gene clusterings in FIG. II-3A-F was obtained and the phenotype confirmed experimentally by the presence of long-chain olefinic hydrocarbons in solvent extracts of growing cells (see section below). Highly likely ole genes were identified in 69 genomes. This was out of 3558 total genomes: Thus, only 1.9% of the genomes examined contained evidence for ole genes using the methods described here. Of the bacterial genomes, 69 out of 1331, or 5.2%, showed bioinformatic evidence for ole genes. The genome analysis included 2143 Eukaryota and 84 Archaea, none of which showed clear evidence of containing an ole gene cluster. This analysis does not rule out that the head-to-head hydrocarbon genes and pathway will be shown to be present in Archae or Eukaryota, merely that our analysis could not identify them with confidence.

TABLE II-1S
Organisms with oleABCD genes. GI identifiers for each gene are given.

| Strain | OleA | OleB | OleC | OleD |
|---|---|---|---|---|
| Arthrobacter aurescens TC1 | 119962129 | OleBC | 119960515 | 119962242 |
| Arthrobacter chlorophenolicus A6 | 220911225 | OleBC | 220911226 | 220911227 |
| Brachybacterium faecium DSM 4810 | 62425589 | OleBC | 237670144 | 237670143 |
| Brevibacterium linens BL2 | 237670145 | OleBC | 62425588 | 62425587 |
| Chloroflexus aggregans DSM 9485 | 118047293 | 118047297 | 118047295 | 118047294 |
| Chloroflexus aurantiacus J-10-fl | 163849058 | 163849062 | 163849060 | 163849059 |
| Chloroflexus sp. Y-400-fl | 187599902 | 187599906 | 187599904 | 187599903 |
| Clavibacter michiganensis subsp. | 170782221 | OleBC | 170782220 | 170782219 |
| Sepedonicus | | | | |
| Colwellia psychrerthraea 34H | 71279747 | 71279056 | 71281286 | 71280771 |
| Congregibacter litoralis KT71 | 88700054 | OleBC | 88705540 | 88705539 |
| Desulfococcus oleovorans Hxd3 | 158522019 | 158522020 | 158522021 | 158522022 |
| Desulfotalea psychrophila LSv54 | 51244593 | 51246484 | 51246483 | 51246482 |
| Desulfuromonas acetoxidans DSM 684 | 95929232 | 95929233 | 95929234 | 95929235 |
| Gamma proteobacterium NOR5-3 | 225089533 | 225089532 | 225089531 | 225089530 |
| Geobacter bemidjiensis Bem | 197118484 | 197118483 | 197118482 | 197118481 |
| Geobacter lovleyi SZ | 189425328 | 189425329 | 189425330 | 189425331 |
| Geobacter sp. FRC-32 | 110599660 | 110599661 | 110599662 | 110599663 |
| Geobacter sp. M21 | 191164328 | 191160706 | 191160707 | 191160708 |
| Geobacter uraniireducens Rf4 | 148264067 | 148264066 | 148264065 | 148264064 |
| Geodermatophilus obscurus DSM | 227405470 | OleBC | 227405471 | 227405473 |
| 43160 | | | | |
| Kineococcus radiotolerans SRS30216 | 152965648 | OleBC | 152965647 | 152965646 |
| Kocuria rhizophila DC2201 | 184200698 | OleBC | 184200697 | 184200696 |
| Kytococcus sedentarius DSM 20547 | 227995171 | OleBC | 227995172 | 227995173 |
| Micrococcus luteus NCTC 2665 | 239917824 | OleBC | 239917825 | 239917826 |
| Moritella sp. PE36 | 149909209 | 149909208 | 149909207 | 149909206 |
| Nakamurella multipartita DSM 44233 | 229225818 | OleBC | 229225819 | 229225820 |
| Opitutaceae bacterium TAV2 | 225164858 | 225164859 | 225155590 | ** |
| Opitutus terrae PB90-1 | 182415091 | 182415090 | 182412680 | 182412679 |
| Pelobacter propionicus DSM 2379 | 118581504 | 118581505 | 118581518 | 118581519 |
| Photobacterium profundum 3TCK | 90413871 | 90413872 | 90413873 | 90413874 |
| Photobacterium profundum SS9 | 54308655 | 54308656 | 54308657 | 54308658 |
| Planctomyces maris DSM 8797 | 149174448 | 149178001 | 149178707 | 149178706 |
| Plesiocystis pacifica SIR-1 | 149918031 | 149918029 | 149918030 | 149918028 |
| Psychromonas ingrahamii 37 | 119945681 | 119945682 | 119945683 | 119945684 |
| Psychromonas sp. CNPT3 | 90408674 | 90408673 | 90408672 | 90408671 |
| Shewanella amazonensis SB2B | 119774319 | 119774320 | 119774321 | 119774322 |
| Shewanella baltica OS155 | 126173784 | 126173785 | 126173786 | 126173787 |
| Shewanella baltica OS185 | 153000075 | 153000076 | 153000077 | 153000078 |
| Shewanella baltica OS195 | 160874697 | 160874698 | 160874699 | 160874700 |
| Shewanella baltica OS223 | 217973959 | 217973958 | 217973957 | 217973956 |
| Shewanella benthica KT99 | 163751382 | 163751383 | 163751967 | 163751968 |
| Shewanella denitrificans OS217 | 91792727 | 91792728 | 91792729 | 91792730 |
| Shewanella frigidimarina NCIMB 400 | 114562543 | 114562544 | 114562545 | 114562546 |
| Shewanella halifaxensis HAW-EB4 | 167624737 | 167624736 | 167624735 | 167624734 |
| Shewanella loihica PV-4 | 127512642 | 127512643 | 127512644 | 127512645 |
| Shewanella oneidensis MR-1 | 24373309 | 24373310 | 24373311 | 24373312 |
| Shewanella pealeana ATCC 700345 | 157962557 | 157962556 | 157962555 | 157962554 |
| Shewanella piezotolerans WP3 | 212636100 | 212636099 | 212636098 | 212636097 |
| Shewanella putrefaciens 200 | 124547320 | 124547321 | 124547322 | 124547323 |
| Shewanella putrefaciens CN-32 | 146292545 | 146292546 | 146292547 | 146292548 |
| Shewanella sediminis HAW-EB3 | 157374649 | 157374650 | 157374651 | 157374652 |
| Shewanella sp. ANA-3 | 117921156 | 117921155 | 117921154 | 117921153 |
| Shewanella sp. MR-4 | 113970883 | 113970882 | 113970881 | 113970880 |
| Shewanella sp. MR-7 | 114048107 | 114048106 | 114048105 | 114048104 |
| Shewanella sp. W3-18-1 | 120599457 | 120599456 | 120599455 | 120599454 |
| Shewanella woodyi ATCC 51908 | 170727499 | 170727498 | 170727497 | 170727496 |
| Stenotrophomonas maltophilia K279a | 190572283 | 190572284 | 190572286 | 190572287 |
| Stenotrophomonas maltophilia R551-3 | 194363945 | 194363946 | 194363948 | 194363949 |
| Stenotrophomonas sp. SKA14 | 254521309 | * | 254522078 | 254520980 |
| Streptomyces ambofaciens DSM40697 | 117164435 | 117164436 | 117164437 | 117164438 |
| Streptomyces ghanaensis ATCC 14672 | 239928261 | 239928262 | 239928263 | 239928264 |
| Xanthomonas axonopodis pv. citri str. | 21241007 | 21241009 | 77748519 | 21241012 |
| 306 | | | | |
| Xanthomonas campestris pv. | 66766567 | 66766571 | 77761077 | 66766574 |
| campestris str. 8004 | | | | |
| Xanthomonas campestris pv. | 21229690 | 21229694 | 77747740 | 21229697 |
| campestris str. ATCC 33913 | | | | |
| Xanthomonas campestris pv. | 188989629 | 188989631 | 188989633 | 188989637 |
| campestris str. B100 | | | | |
| Xanthomonas oryzae KACC10331 | 58583832 | 58583836 | 122879327 | 58583839 |
| Xanthomonas oryzae MAFF 311018 | 84625635 | 84625639 | 84625641 | 84625642 |
| Xanthomonas oryzae pv. oryzicola | 166710197 | 166710199 | 166710201 | 166710202 |
| BLS256 | | | | |
| Xanthomonas oryzae PXO99A | 188574836 | 188574832 | 188574829 | 188574828 |

* There is a large area with no gene identified between oleA and oleC homologs. When the genome is searched with the oleB homolog DNA sequence from Stenotrophomonas maltophilia K279a, it hits region with 93% identity. Therefore the nucleotide sequence is there but not predicted.
** Unfinished genome - OleABC are similar to Opitutus terrae PB90-1 (58-71% identity) but the OleD homolog is not adjacent to OleC in the genome, though many OleD homologs are present in the genome

Hydrocarbon identification. It could not be inferred from sequence analysis alone whether all of the gene configurations would give rise to hydrocarbon products. In this context, at least one organism from each class (A-F) of FIG. II-3 was tested directly for long-chain olefin biosynthesis. In previous studies (Albro et al. 1969. Biochemistry. 8:394-405; Beller et al. Appl. Environ. Micro. 76:1212-1223; Frias et al. 2009. Appl. Environ. Microbiol. 75:1774-1777; Sukovich et al. 2010. Appl. Environ. Microbiol.; Tornabene et al. 1967. J. Bacteriol. 94:333-343), olefins were produced under all growth conditions for all of the organisms tested; olefin production appears to be constitutive. In this context, each strain was grown under optimum conditions for that strain as described in Table II-1 and the Materials and Methods. From each organism, non-polar material was extracted with solvent and analyzed by chromatography and mass spectrometry. Controls were conducted with solvent blanks and organisms previously described not to produce head-to-head hydrocarbons (Wackett et al. 2007. Appl. Environ. Microbiol. 73:7192-7198) to exclude olefins that were derived from solvents or workup procedures. This study showed that bacteria from different types of gene clusterings shown in FIG. II-3 produced hydrocarbons in direct experimental tests (FIG. II-4 and Table II-3). Different hydrocarbons were produced but all were long chain (>C23) and contained at least one double bond, consistent with their formation by a head-to-head coupling of fatty acyl groups.

TABLE II-3
Compilation of head-to-head olefins produced by different bacteria

| | Total hydrocarbons | | Predominant hydrocarbon | |
|---|---|---|---|---|
| | # hydrocarbons | Size | Mass spectrum¹ | Chemical |
| Microorganism | detected | range | (m/z) | formula |
| Chloroflexus aurantiacus J-10-fl | 1 | C₃₁H₅₈ | 430, 303 | C₃₁H₅₈ |
| Kocuria rhizophila DC2201 | 12 | C₂₄H₄₈-C₂₉H₅₈ | 478, 348 | C₂₇H₅₄ |
| Brevibacterium fuscum ATCC 15993 | 9 | C₂₇H₅₄-C₂₉H₅₈ | 406, 376 | C₂₉H₅₈ |
| Xanthomonas campestris pv. campestris | 15² | C₂₈H₅₆-C₃₁H₅₈ | 402, 303 | C₂₉H₅₄ |
| Shewanella oneidensis MR-1 | 1 | C₃₁H₄₆ | 418, 281 | C₃₁H₄₆ |
| Shewanella putrefaciens CN-32 | 1 | C₃₁H₄₆ | 418, 281 | C₃₁H₄₆ |
| Shewanella baltica OS185 | 1 | C₃₁H₄₆ | 418, 281 | C₃₁H₄₆ |
| Shewanella frigidimarina NCIMB 400 | 1 | C₃₁H₄₆ | 418, 281 | C₃₁H₄₆ |
| Shewanella amazonensis SB2B | 1 | C₃₁H₄₆ | 418, 281 | C₃₁H₄₆ |
| Shewanella denitrificans OS217 | 1 | C₃₁H₄₆ | 418, 281 | C₃₁H₄₆ |
| Colwellia psychrerythraea 34H | 1 | C₃₁H₄₆ | 418, 281 | C₃₁H₄₆ |
| Geobacter bemidjiensis Bem | 1 | C₃₁H₄₆ | 418, 281 | C₃₁H₄₆ |
| Opitutaceae bacterium TAV2 | 1 | C₃₁H₄₆ | 418, 281 | C₃₁H₄₆ |
| Planctomyces maris DSM 8797 | 1 | C₃₁H₄₆ | 418, 281 | C₃₁H₄₆ |

¹Identifying ions for the predominant long-chain olefin
²Number readily identifiable by gas chromatography-mass spectrometry

Shewanella amazonensis Sb2B, isolated from the Amazon River delta off of the coast of Brazil (Venkateswaran et al. 1998. Int. J. Syst. Bacteriol. 48:965-972) contained recognizable ole genes. It produced a single product with a carbon chain length of 31 and 9 double bonds (C₃₁H₄₆). The GC retention time and the mass spectrum indicated that the compound was identical to that produced by S. oneidensis strain MR-1 that had been described above. The hydrocarbon in S. oneidensis MR-1 is the C₃₁ polyolefin 3,6,9,12,15,19,22,25,28-hentriacontanonaene. Additional Shewanella strains were tested in this study and all produced the C₃₁ polyolefin as the only discernible hydrocarbon (Table II-3).

Colwellia psychreryhtraea is an obligate psychrophile that grows at temperatures below 0° C., Geobacter bemidjiensis Bem was isolated from a petroleum-contaminated aquifer sediment, Opitutaceae TAV2 is a member of the phylum Verrucomicrobia but not well-studied (Schmidt, T. D. Nov. 23, 2009. Genome project: Opitutaceae bacterium TAV2. World Wide Web, URL=http://genome.jgi-psf.org/opiba/opiba.info.html), and Planctomyces maris DSM8797 is in the phylum Planctomycetes, isolated from the open ocean (Bauld et al. 1976. J. Gen. Microbiol. 97:45-55). Despite the great phylogenetic and ecological diversity of these bacteria, they all produced a single hydrocarbon product with the same retention time (20.2 min) and mass spectrum, consistent with its identity as 3,6,9,12,15,19,22,25,28-hentriacontanonaene (FIG. II-4 and Table II-3).

A closely migrating, but clearly distinct, hydrocarbon product was produced by Chloroflexus aurantiacus strain J-10-fl (FIG. II-4), a bacterium isolated from hot springs that grows optimally at 55° C. (Pierson et al. 1974. Arch. Microbiol. 100:5-24). The Chloroflexus hydrocarbon migrated more slowly on the GC column (20.4 min) and the mass spectrum indicated a chemical formula of C₃₁H₅₈, consistent with a hydrocarbon consisting of 31 carbon atoms and three double bonds. These data are consistent with previous reports that identified hentriaconta-9,15,22-triene (C₃₁H₅₈) in microbial mats (van der Meer et al. 1999. Org. Geochem. 30:1585-1587) and being formed by Chloroflexus spp. in pure culture (van der Meer et al. 2001. J. Biol. Chem. 276:10971-10976).

Kocuria rhizophila strain DC2201 was isolated for its ability to withstand organic solvents (Fujita et al. 2006. Enzyme Microb. Technol. 39:511-518) and its complete genome sequence was reported by Hiromi et al. 2008. J. Bacteriol. 190: 4139-4146. Here it was shown to produce multiple olefinic hydrocarbon products that ranged from 24 to 29 carbon atoms (Table II-3). Each identified compound contained one double bond. The clusters of compounds eluting at approximately 16 min, 16.8 min, 17.5 min and 19 min (FIG. II-4) represent isomeric clusters of C₂₅, C₂₆, C₂₇, and C₂₉ chain lengths, respectively, based on mass spectrometry. This type of hydrocarbon cluster resembled, but was not identical to, those found in Arthrobacter (Frias et al. 2009. Appl. Environ. Microbiol. 75: 1774-1777) and Micrococcus species (Albro et al. 1969. Biochemistry. 8:394-405; Beller et al. Appl. Environ. Micro. 76:1212-1223; Tornabene et al. 1970. Lipids. 5:929-934) that have been studied previously. The major compounds in Kocuria analyzed here contained 25 and 27 carbon atoms. Another Actinobacterial strain that had not yet been tested for the presence of head-to-head hydrocarbons, Brevibacterium fuscum ATCC15993, similarly produced isomeric clusters of hydrocarbons but in the range of 27 to 29 carbon atoms (Table II-3).

The most extensive array of hydrocarbon products from those organisms tested here was observed with Xanthomonas campestris (FIG. II-4 and Table II-3), a bacterium that causes a range of plant diseases (Burnes et al. 2000. Appl. Environ. Microbiol. 66:5201-5205; Vauterin et al. 1995. Int. J. Syst. Bacteriol. 45:472-489). X. campestris produced hydrocarbons with chain length of C₂₈, C₂₉, C₃₀ and C₃₁. Based on the mass spectra, hydrocarbons containing one, two, or three double bonds could be identified. There was additional structural complexity that was likely due to isomerization that could arise from different types of methyl-branching at the hydrocarbon termini. The complexity of the mixture precluded precise structural determinations that would require the availability of synthetic standards.

Negative controls were run to rule out artifacts that could result, for example, from hydrocarbon contamination external to the cells (Wackett et al. 2007. Appl. Environ. Microbiol. 73:7192-7198). The most telling experimental results were obtained with Geobacter sulfurreducans PCA, an organism closely related to G. bemidjiensis Bem but suggested from bioinformatics analysis in this study to contain an oleA homolog with a different function (FIG. II-2). Olefinic hydrocarbons were not detected in G. sulfurreducans. Additionally, long-chain olefinic hydrocarbons were not detected in cultures of E. coli K12 or Vibrio furnissii M1, both of which were determined not to contain ole genes using the bioinformatics analysis described here.

Most previous studies had investigated bacterial head-to-head hydrocarbon biosynthesis in members of the Actinobacteria that includes Micrococcus (Albro et al. 1969. Biochemistry. 8:394-405; Albro et al. 1969. Biochemistry. 8:1913-1918; Beller et al. Appl. Environ. Micro. 76:1212-1223) and Arthrobacter (Frias et al. 2009. Appl. Environ. Microbiol. 75:1774-1777). Long-chain olefinic hydrocarbons had also been demonstrated in Stenotrophomonas maltophilia (Suen et al. 1988. J. Ind. Microbiol. 2:337-48), a member of the phyla Proteobacteria. The present study showed additional Actinobacteria (Brevibacterium) and Proteobacteria (Geobacter sp.) produce head-to-head hydrocarbons. In addition, members of the phyla Verucomicrobia, Planctomyces, and Chloroflexi were shown to contain bona fide ole genes and to produce olefinic hydrocarbons. This greatly expanded the phylogenetic diversity demonstrated experimentally to produce head-to-head olefinic hydrocarbons and revealed the type(s) of hydrocarbon produced. The latter could not be discerned from the ole gene sequences alone based on previous studies. The present study begins to make a link between one of the Ole protein sequences with the hydrocarbon(s) produced as discussed in the section below.

OleA has a major role in determining the type of head-to-head products formed. The different long-chain olefinic hydrocarbons identified in this and other studies showed variable chain lengths and degrees of unsaturation. This could be determined largely by the fatty acid composition within the cell, by the substrate specificity of the Ole proteins, by other proteins, or by some combination of these factors. To begin to investigate this, S. oneidensis MR-1 strains with different oleA gene contents were grown identically and tested for hydrocarbon content. The S. oneidensis MR-1 strains contained respectively: (A) the native Shewanella oleA gene only, (B) the native Shewanella oleA gene plus a Stenotrophomonas oleA gene, (C) no oleA gene, (D) the Stenotrophomonas oleA gene in a Shewanella oleA deletion strain and (E) the Stenotrophomonas oleA gene in a Shewanella oleABCD deletion strain. Each strain (A-E) was grown under the same conditions of medium, temperature and aeration. Each strain was harvested and extracted the same way. Each extract was subjected to the same chromatographic procedures.

The chromatograms shown in FIG. II-5 suggested that the product composition is strongly influenced by the oleA gene. In the same cell type, with cells grown under the same conditions and therefore likely having the same fatty acid precursor pools, the product distribution was completely different when oleA genes from different organisms were present. When oleA genes native to Shewanella and Stenotrophomonas were produced in the same cell, the products were additive to what was found with either alone (FIG. II-5B). Moreover, the Stenotrophomonas oleA gene, in the absence of the native oleBCD genes, was sufficient to make products of fatty acid head-to-head condensation (FIG. II-5E). This has implications for the mechanism of olefin biosynthesis and will be discussed in more detail below.

When the Shewanella oleA gene was present, the cells made compound I (FIG. II-5A&B) that had been previously identified as a polyolefin containing nine double bonds derived from an intermediate in the polyunsaturated fatty acid biosynthetic pathway (Sukovich et al. 2010. Appl. Environ. Microbiol.). The presence of the Stenotrophomonas oleA gene led to the formation of new products of fatty acid condensation. All of the later-eluting compounds labeled II-V in FIG. II-5 were ketones. This was apparent from mass spectrometry based on: (a) the parent ions, (b) prominent fragment ions and (c) comparison to an authentic long-chain ketone standard. From known fragmentation of alkyl ketones, and the observed fragmentation with standard 14-heptacosanone, the major fragments expected were R—CH₂—C═O. In the case of 14-heptacosanone, the carbonyl group is directly in the middle and fragmentation at either side on the carbonyl functionality yields a fragment of m/z 211 and this was observed experimentally using GC-MS. Compound II (FIG. II-5) showed fragments of m/z 223 and 225 and a parent ion of m/z 420 consistent with a compound containing a carbonyl functionality directly in the middle of a C₂₉ chain with 14 saturated carbon atoms on one side and a C₁₄ chain with one double bond on the other. Compound III showed a fragment with m/z 223 and a parent ion of m/z 418. This mass spectrum is consistent with a compound containing a carbonyl functionality directly in the middle of a C₂₉ chain flanked by two C₁₄ chains each containing one double bond. Compound IV showed a fragment with m/z 225 and a parent ion of m/z 422. This mass spectrum is consistent with a compound containing a carbonyl functionality directly in the middle of a C₂₉ chain flanked by two saturated C₁₄ chains. Compound V had a very similar mass spectrum as Compound II. This suggested that it a positional isomer of compound II and consisted of hydrocarbon chains with one double bond and a saturated chain, respectively, linked together by a carbonyl functionality.

The data above are consistent with a fatty acid condensation between specific saturated and monounsaturated fatty acids. In separate experiments in which the Shewanella oleABCD deletion mutant was complemented with the Shewanella oleA gene, a compound with m/z 434 was obtained. This mass is consistent with a C₃₁ compound containing one ketone functionality and eight carbon-carbon double bonds. The structure was confirmed by chemical modification. After hydrogenation, the compound had a parent ion of m/z 450 with a major fragment ion of m/z 239. This had the expected parent ion and major ion fragment for 16-hentriacontanone. Like the results shown in FIG. II-5A-E, this result was consistent with an oleA gene product causing specific condensation of two fatty acids. The Shewanella OleA showed selectivity for polyunsaturated fatty acids while the Stenotrophomonas OleA showed selectivity for saturated or mono- or di-unsaturated fatty acids.

A mechanism to explain the formation of ketones in the presence of oleA genes alone is proposed in a section below. In total, these data highlight a potentially strong selectivity difference between OleA proteins from Shewanella and Stenotrophomonas, respectively. The observations here showing that different oleA genes exert a strong influence on fatty acid condensation has implications for potential use of different ole genes to produce targeted hydrocarbon products commercially. Certain hydrocarbon products may be more desirable for industrial applications. In this context, a knowledge of OleA protein specificity would be critical in efforts to control product structure.

Olefin type in divergent bacteria tracks most closely with OleA sequence. Very different types of olefin products were observed in wild-type bacteria, containing a range from one to nine double bonds. Most bacteria in this study made exclusively the nonaene polyolefin previously identified in Shewanella. Data was presented in a previous study indicating that the C₃₁ nonaene compound was derived from polyunsaturated fatty acid precursors (Sukovich et al. 2010. Appl. Environ. Microbiol). However, at most 10% of the fatty acids produced by Shewanella and other bacterial strains are polyunsaturated (Abboud et al. 2005. Appl. Environ. Microbiol. 71:811-6; Hedrick et al. 2009. J. Ind. Microbiol. Biotechnol. 36:205-9; Ivanova et al. 2004. J. Syst. Evol. Microbiol. 54:1773-1788; Lee et al. 2008. J. Microbiol. Biotechnol. 18:1869-73). This strongly suggested that Ole enzymes must show selectivity in condensing certain fatty acids and not others. In light of the observations with oleA genes from Shewanella and Stenotrophomonas (FIG. II-5), the OleABCD protein sequences were analyzed to see if, amongst the diverse bacteria analyzed here, Ole protein sequence relatedness correlated with the type of olefin produced by the cell.

Network clustering software was used to visualize the multi-dimensional relatedness of different sequences, as this method has been shown to be superior to trees for visualizing protein sequence relatedness (Meng et al. 2006. BMC Bioinformatics. 7:339-349). The method makes an all-by-all blastp library of a sequence set. From this data, a network diagram is created in which the nodes represent protein sequences and the edges represent a blast linkage that connects the two proteins. A shorter edge represents a lower e-score (greater relatedness). For example, an e-value cutoff of e⁻⁷³ was used in FIG. II-6. If the e-value of any pairwise comparison is lower (more related) than e⁻⁷³, then the sequences (circles/nodes in FIG. II-6) are connected by a line. Nodes that are not connected, or connected to fewer other nodes, are more divergent sequences. In this way, the network representation allows visualizations of connectivity more fully than protein tree analyses.

The network sequence analysis conducted for 17 each OleA, OleB, OleC and OleD sequences are shown in FIG. II-6 (see FIG. II-6S for more detailed clustering experiments). Those 17 were selected because all had been experimentally tested, shown to produce olefinic hydrocarbons, and the hydrocarbon products were identified. The top left side of FIG. II-6 readily shows that ten of the OleA proteins cluster together (having all pairwise comparisons with e-values less than e⁻⁷³) and all produce the single polyolefinic hydrocarbon that is derived from polyunsaturated fatty acids. One explanation for this, that we favor based on the other data presented, is that the OleA proteins in Shewanella, Geobacter, Planctomyces and Opitutacae specifically condense polyunsaturated fatty acids but do not condense the larger pool of more highly saturated fatty acids found in these classes of bacteria (Choo et al. 2007. Int. J. Syst. Evol. Microbiol. 57:532-7; Hedrick et al. 2009. J. Ind. Microbiol. Biotechnol. 36:205-9; Ivanova et al. 2004. J. Syst. Evol. Microbiol. 54:1773-1788; Kulichevskaya et al. 2007. Int. J. Syst. Evol. Microbiol. 57:2680-7).

FIG. II-6A also shows that the OleA proteins that make moderately saturated head-to-head olefins cluster differently than the OleA found in bacteria that produce the polyolefinic hydrocarbon. For example, Chloroflexus aurantica was known to make a C₃₁ triene hydrocarbon (van der Meer et al. 1999. Org. Geochem. 30:1585-1587) and that was confirmed in this study. A C₃₁ triene would derive from the head-to-head condensation of two monounsaturated fatty acids. Chloroflexus aurantica makes predominantly C₁₆ and C₁₈ saturated fatty acids (van der Meer et al. 2001. J. Biol. Chem. 276:10971-10976). The most obvious explanation is that the head-to-head biosynthetic pathway shows selectivity for only certain fatty acids within Chloroflexus.

Since the Ole-mediated head-to-head condensation process shows selectivity, it was investigated which Ole protein sequence networks clustered most strongly with the type of head-to-head olefin formed. FIG. II-6 parts A, B, C, and D represent the clustering networks of OleA, OleB, OleC and OleD, respectively. For OleA (FIG. II-6A), sequence relatedness tracks with the type of olefinic hydrocarbon produced. For OleB, C, and D (FIG. II-6B,C, and D), the sequences cluster differently and are less reflective of the olefinic hydrocarbon structure. This is perhaps most apparent with OleB (FIG. II-6B).

With the cluster represented by the OleABCD sequences from the Actinobacterial genera Arthrobacter, Kocuria and Micrococcus, it was not possible to discern selectivity. The olefinic hydrocarbons produced are methyl-branched and the major fatty acids in Arthrobacter and Microccus are methyl-branched (Tornabene et al. 1967. J. Bacteriol. 94:333-343; Unell et al. 2007. FEMS Microbiol. Lett. 266:138-143). The OleA proteins in the Actinobacterial branch may be non-selective, or the proteins may have evolved selectivity that mirrors the major fatty acid types produced by the cell.

OleA potential mechanisms. The observation that Shewanella OleA (FIG. II-5), Stenotrophomonas OleA (FIG. II-5), and other OleA proteins (FIG. II-6) confer fatty acid substrate selectivity is consistent with OleA catalyzing the first reaction in head-to-head hydrocarbon formation. An alternative proposal has been advanced in which several (3-oxidation steps precede the OleA-catalyzed condensation reaction and the reaction is coincident with the decarboxylation step (Beller et al. Micrococcus luteus. Appl. Environ. Micro. 76:1212-1223). That mechanism was supported by two observations, the requirement for E. coli cell-free extract to support in vitro olefin synthesis and sequence alignments of the Micrococcus luteus OleA with E. coli FabH. The latter enzyme catalyzes a decarboxylative fatty acyl (Claisen) condensation reaction. OleA proteins show the highest percent sequence identity with thiolase superfamily members like FabH that catalyze decarboxylative Claisen condensations.

This present study offers an alternative mechanism. As illustrated in Table II-2, the thiolase superfamily contains several members that catalyze non-decarboxylative fatty acyl condensation reactions, for example the biosynthetic thiolase involved in PHB biosynthesis (Davis et al. 1987. J. Biol. Chem. 262:82-9) and 3-hydroxyl-3-methylglutaryl-CoA synthase (HMG-CoA synthase) (Steussy et al. 2006. Biochemistry. 45:14407-14). The latter enzyme, and other non-decarboxylative thiolase superfamily enzymes share the same highly conserved residues with those of OleA and FabH (FIG. II-1). The decarboxylative and non-decarboxylative thiolase superfamily proteins use these residues in an analogous manner to acylate a cysteine and then attack the bound acyl group with an enzyme generated carbanion (Haapalainen et al. 2005. Trends Biochem. Sci. 31:64-71). The differences in mechanism are subtle. Thus, sequence arguments cannot rule in or out decarboxylative versus non-decarboxylative mechanisms for OleA proteins.

Moreover, the mechanism proposed by Beller, et al. for OleA is not analogous to that catalyzed by FabH. FabH acts on condensing a fatty acyl group containing an α-carboxy group and this activation mechanism is not shown in the proposed mechanism (Beller et al. Appl. Environ. Micro. 76:1212-1223). Instead, the authors propose a series of steps catalyzed by unidentified enzymes to generate a β-ketoacyl chain that then reacts in condensation with release of coenzyme A and carbon dioxide. To our knowledge, there is no reaction analogous to this catalyzed by a known member of the thiolase superfamily.

An alternative mechanism would be for OleA to catalyze a non-decarboxylative Claisen condensation directly analogous to the reaction catalyzed by biosynthetic thiolases that function in PHB (Davis et al. 1987. J. Biol. Chem. 262:82-9) and steroid synthesis (Haapalainen et al. 2005. Trends Biochem. Sci. 31:64-71). Both biosynthetic and catabolic thiolases show free reversibility so dozens of enzymes in the thiolase superfamily are already known to catalyze this general reaction. While the equilibrium constant for the biosynthetic direction is typically unfavorable, subsequent steps can pull the equilibrium as occurs in PHB and steroid biosynthesis.

The product data are also suggestive that OleA catalyzes the first step in head-to-head hydrocarbon biosynthesis. The product selectivity shown in this study to arise from the oleA gene would be unusual if the OleA protein was in the middle of the biosynthetic pathway as proposed by Beller, et al. (Beller et al. Micrococcus luteus. Appl. Environ. Micro. 76: 1212-1223). Biosynthetic pathways are typically controlled at the first committed step in the pathway (Gunnarsson et al. 2004. Adv. Biochem. Engin. Biotech. 88:137-178; Lehninger et al. 1978. Biochemistry: The Molecular Basis of Cell structure and Function. Worth Publishers, New York, N.Y.). The mechanism proposed by Beller et al. (Beller et al. Micrococcus luteus. Appl. Environ. Micro. 76: 1212-1223) requires additional enzymes to generate the 1,3-diketone that is proposed to undergo OleA-catalyzed condensation with a second fatty acyl chain. Those putative genes were searched for in the present study. The genes would need to be present in organisms producing head-to-head hydrocarbons and they might be expected to be contiguous, at least in some organisms, to the other genes encoding enzymes in the same metabolic pathway. However, we found that the gene regions contiguous to the oleABCD gene clusters were very different from organism to organism, and we failed to identify genes encoding enzymes that act to oxidize an acyl chain to generate a β-ketoacyl chain. This suggests that the OleABCD proteins may be sufficient for ketone and olefin biosynthesis.

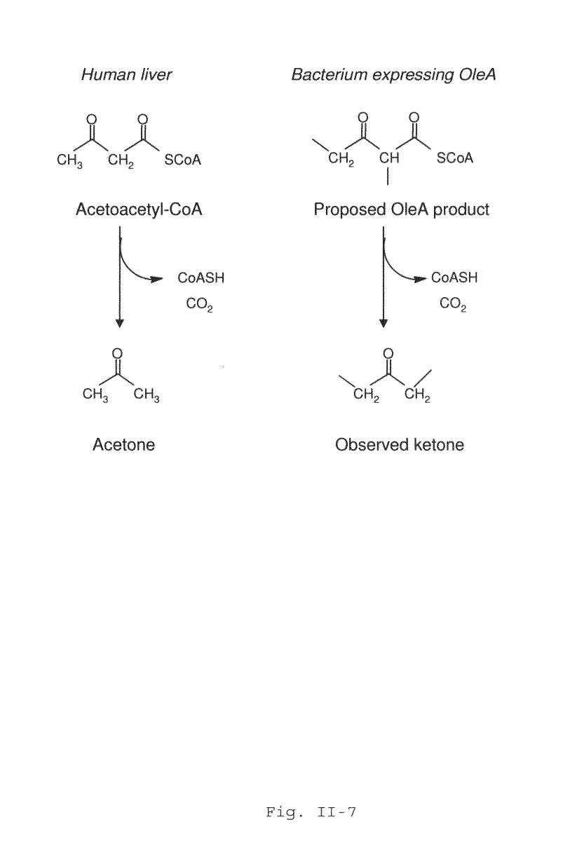

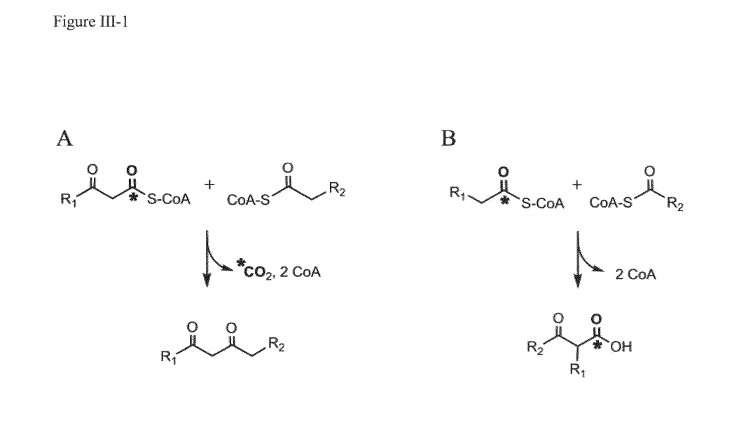

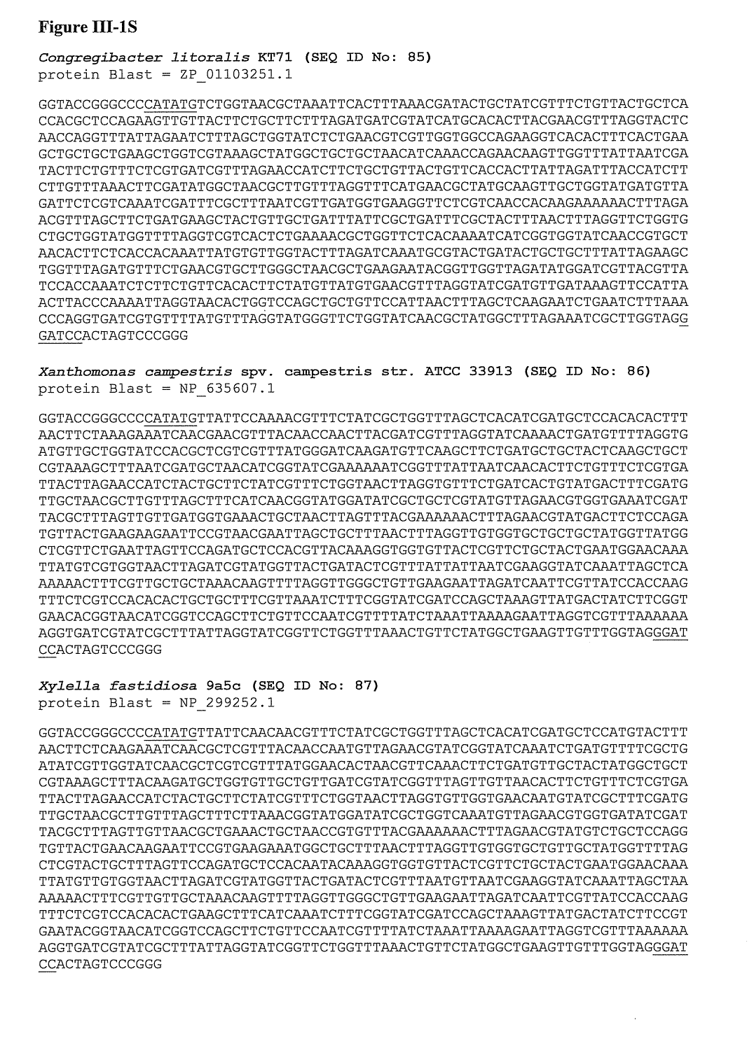

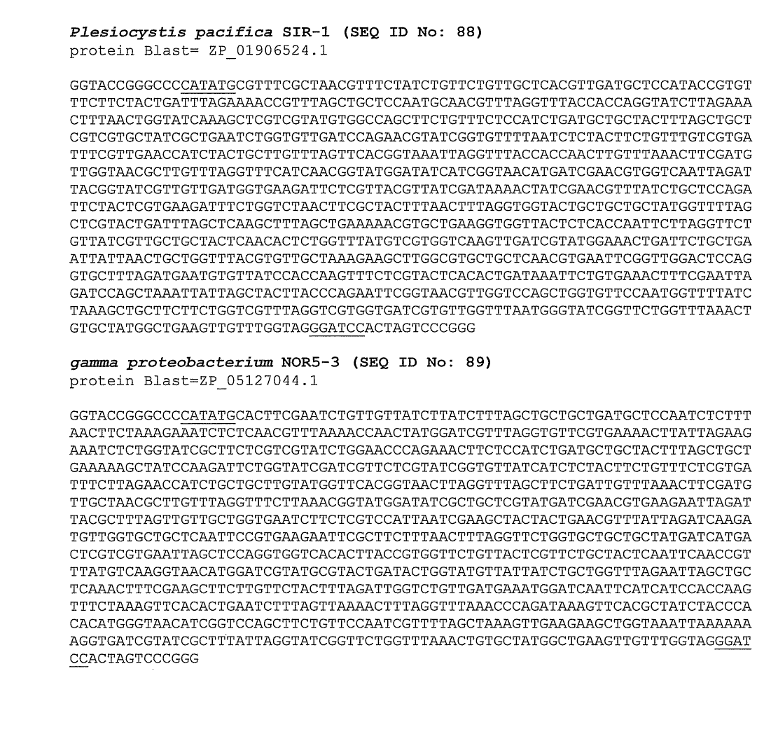

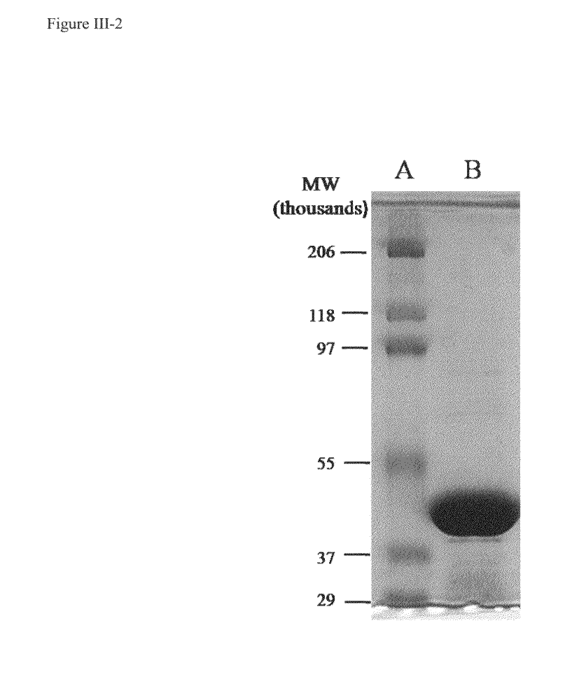

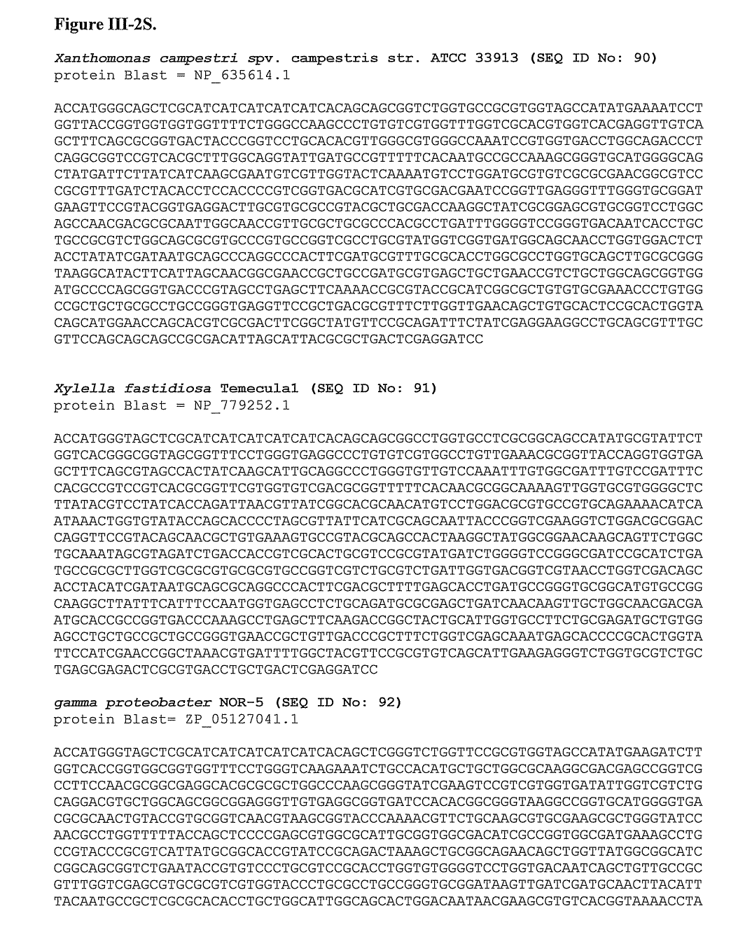

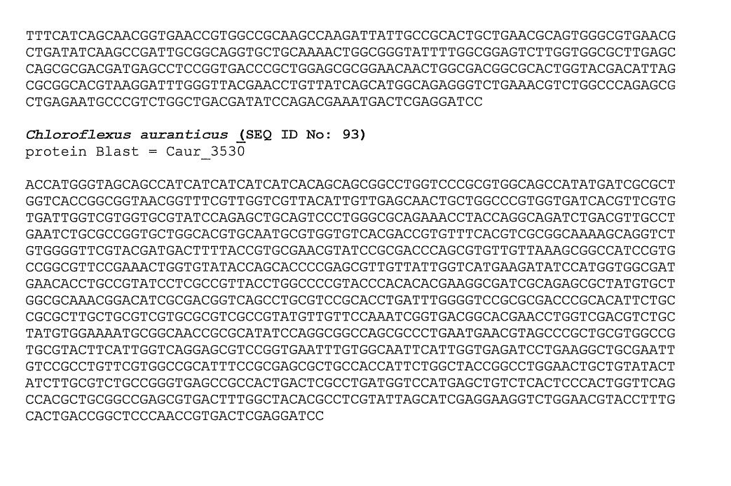

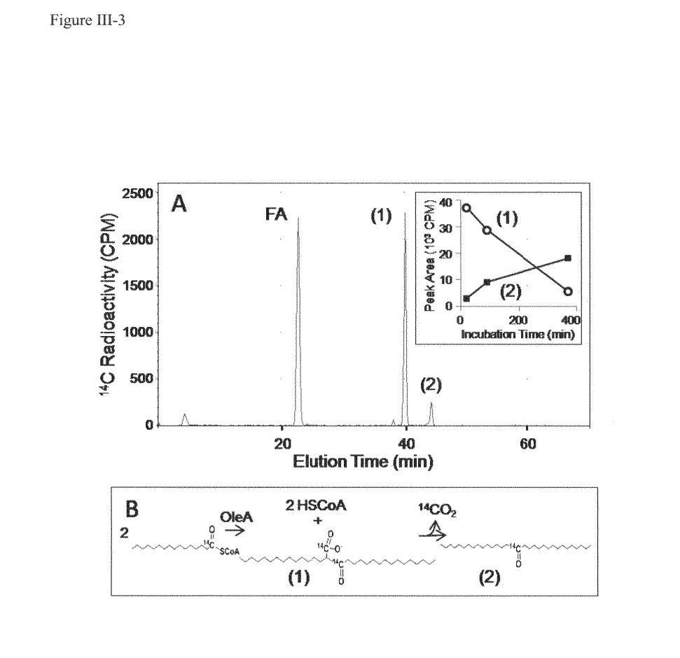

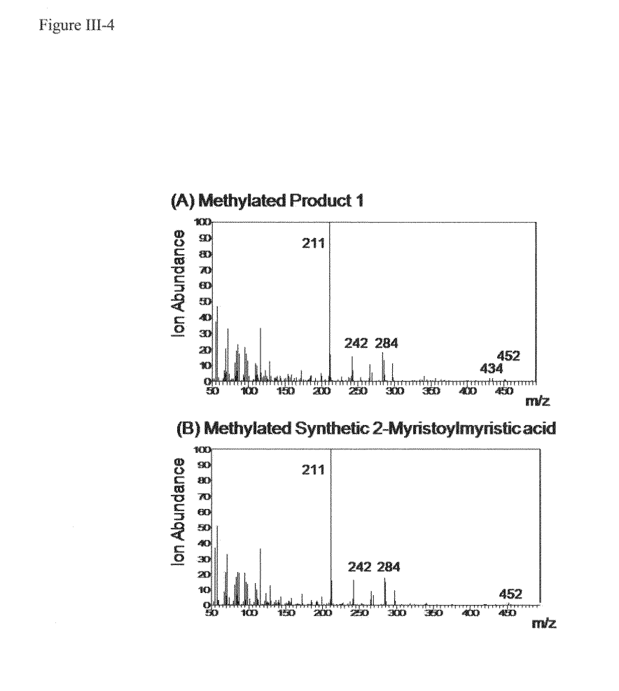

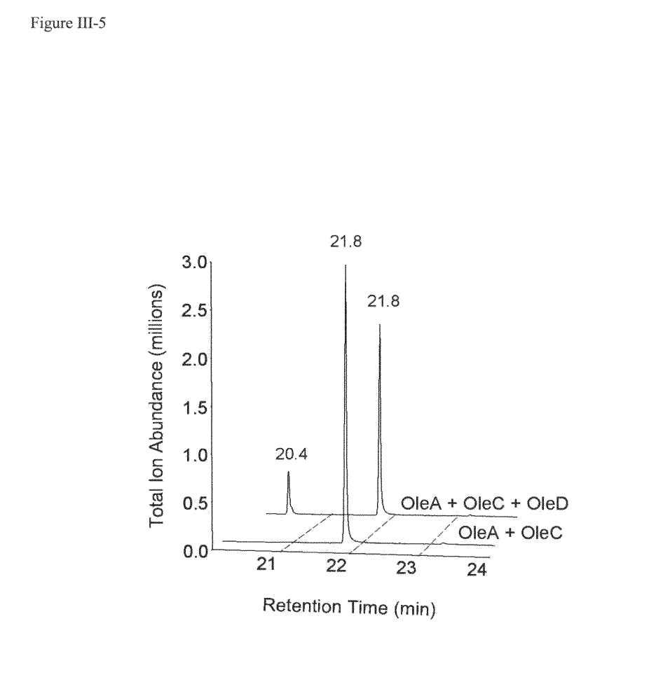

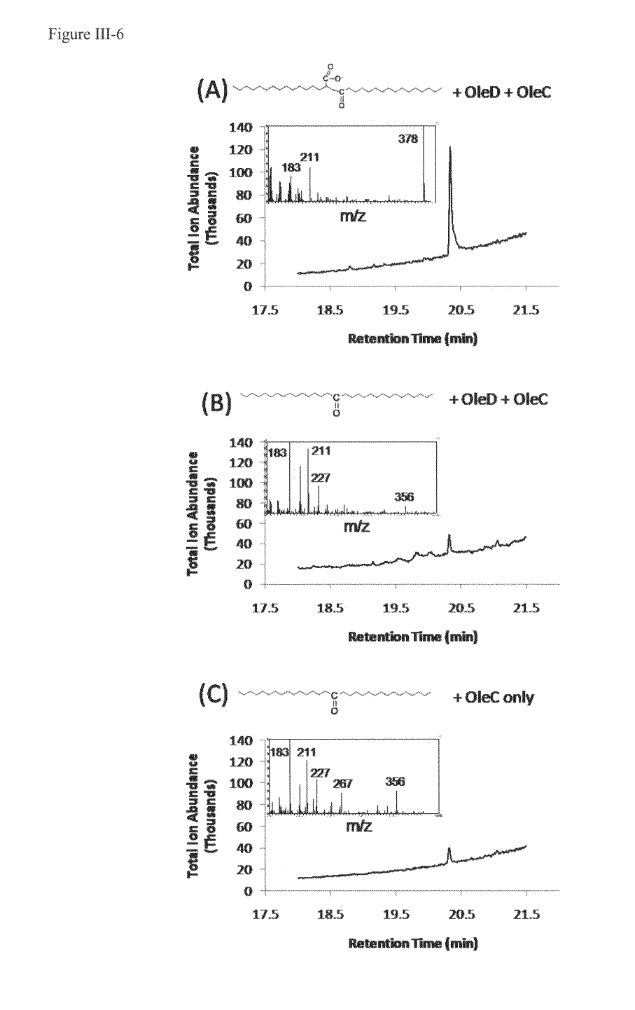

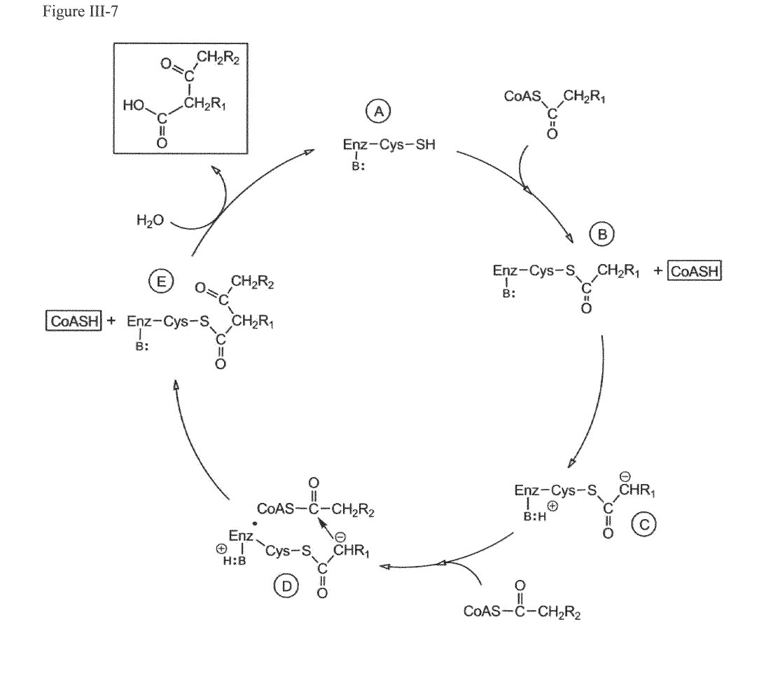

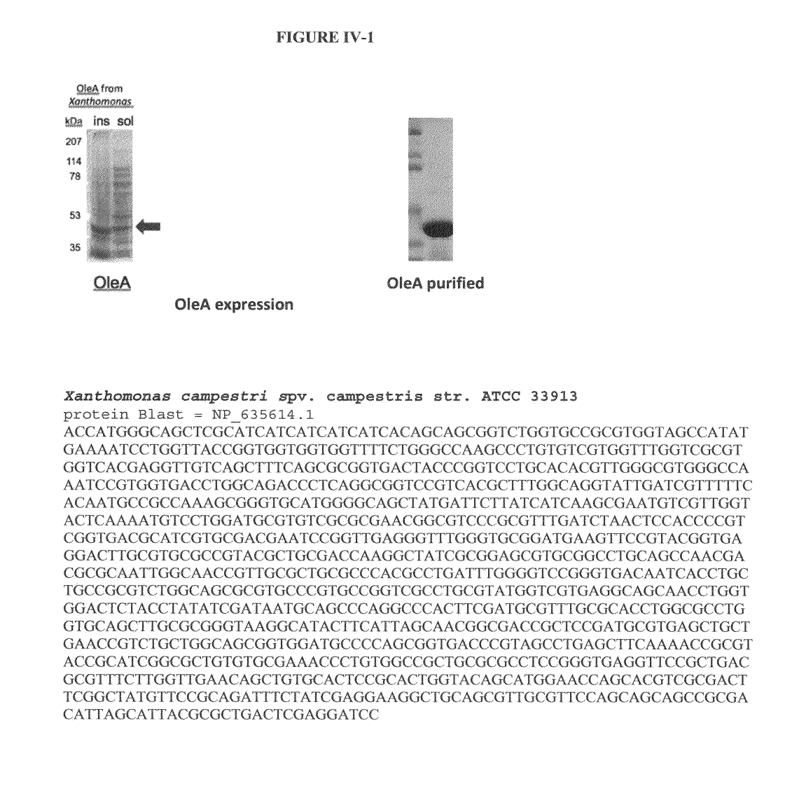

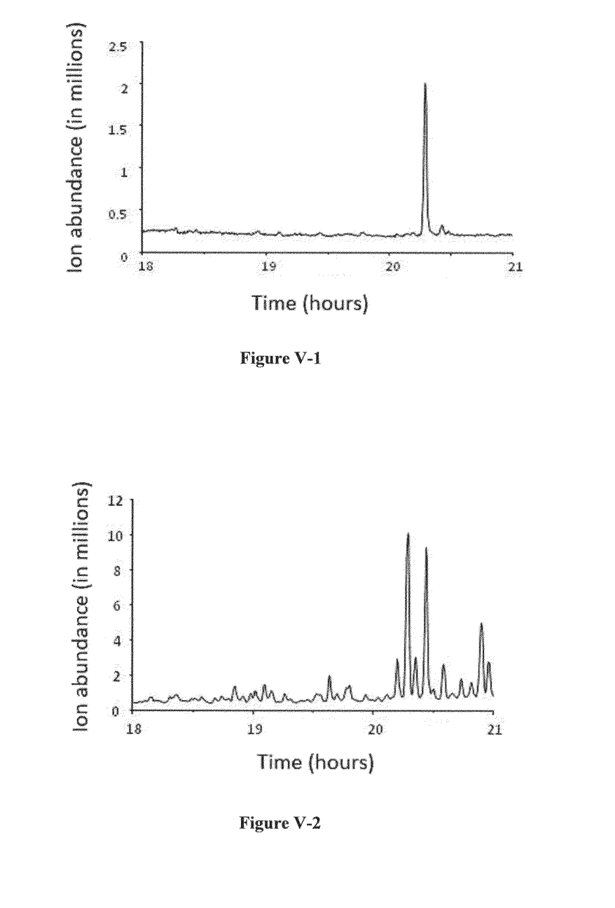

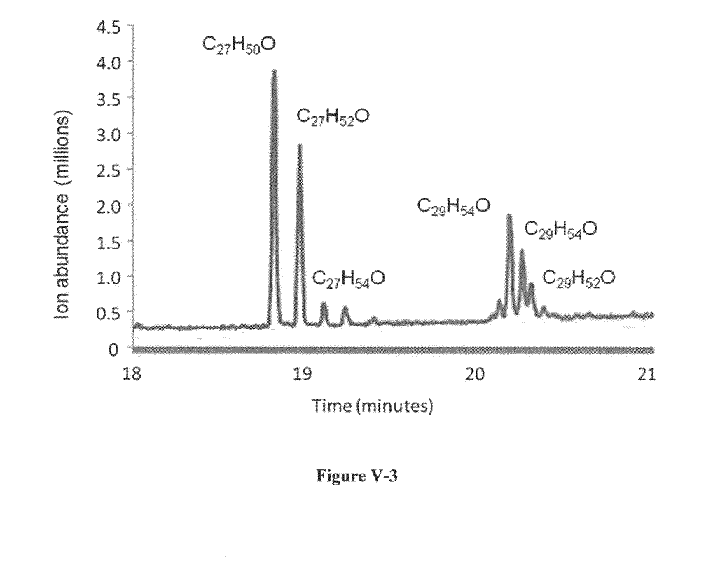

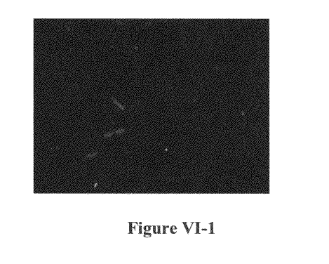

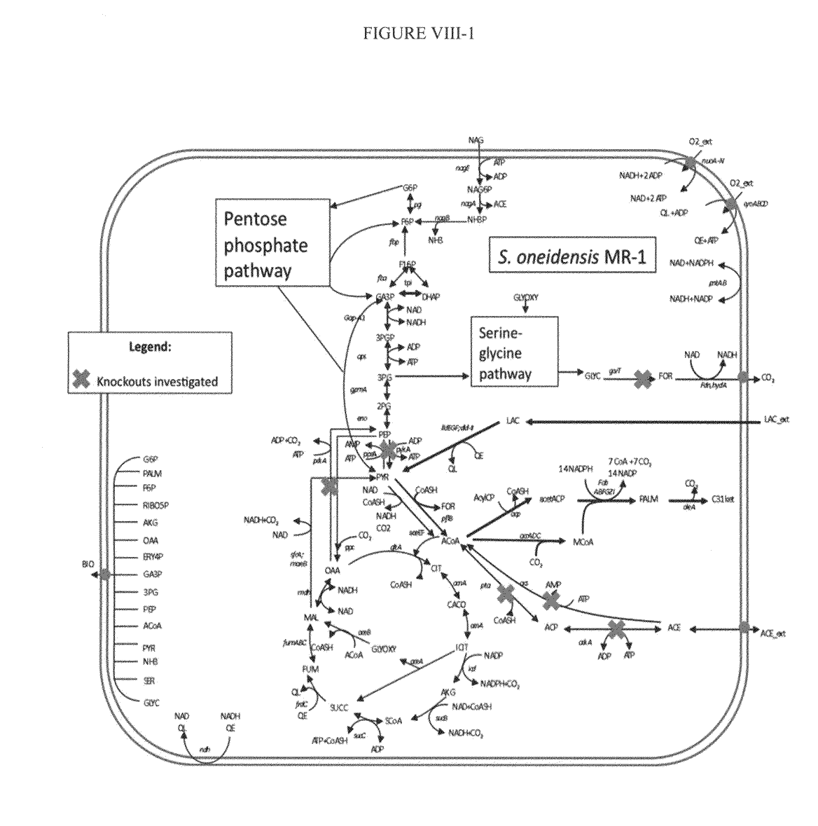

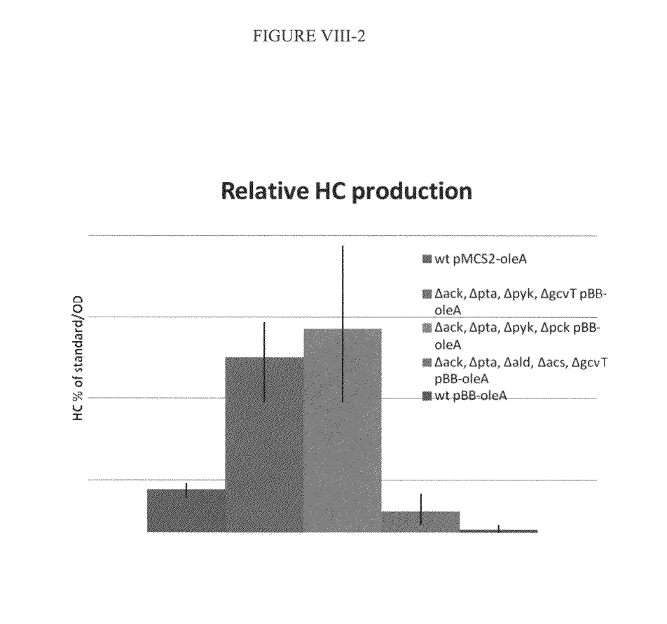

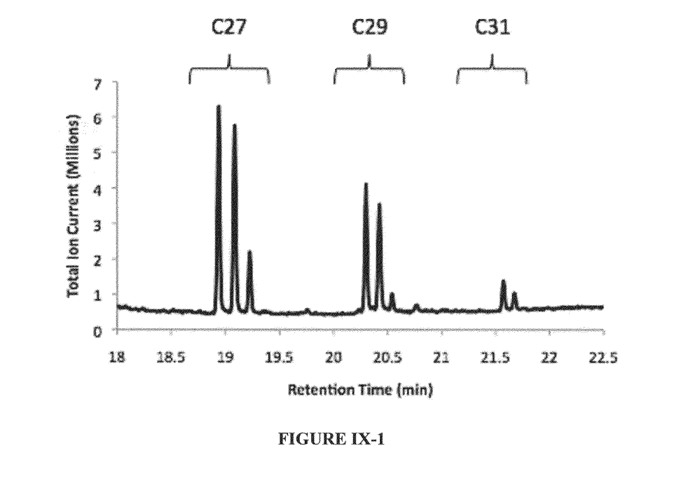

Unlike the previous study (Beller et al. Appl. Environ. Micro. 76:1212-1223), a non-decarboxylative, thiolytic type of fatty acyl condensation is proposed here. The non-decarboxylative type of mechanism would explain the observed formation of ketones with OleA in vivo and in vitro (Albro et al. 1969. Biochemistry. 8:394-405; Beller et al. Appl. Environ. Micro. 76:1212-1223; Friedman et al. 2008. International Patent Application WO 2008/147781; Friedman et al. 2008. International Patent Application WO 2008/113041; 62) and that the proposed 1,3-dione intermediate (Beller et al. Appl. Environ. Micro. 76: 1212-1223) has not been observed to date. Ketone formation following a direct OleA-catalyzed non-decarboxylative coupling of fatty acyl chains is chemically plausible and biochemically precedented (FIG. II-8). This is reminiscent of the formation of acetone in humans via acetoacetyl-CoA (Hird et al. 1962. Biochem. J. 84:212-216). Acetoacetyl-CoA is a beta-ketoacyl compound, as is the thiolytic product of the OleA reaction that we propose (FIG. 11-7). In human liver, excess acetoacetyl-CoA can give rise to acetoacetate that is known to undergo spontaneous decarboxylation to acetone. The spontaneous decarboxylation of β-keto acids of this type has been known for more than 80 years and is quite facile (Pedersen et al. 1929. J. Am. Chem. Soc. 51:2098-2107). In a similar manner, we propose that the product(s) of the OleA reaction, if not acted upon by OleBCD, could undergo thioester hydrolysis either spontaneously (Fredslund et al. 2006. J. Mol. Biol. 361:115-127) or enzymatically, and decarboxylation to generate a ketone(s). Note that the acyl-CoA compounds shown in FIG. 11-7 are directly analogous. They can both arise from thiolytic condensation of either acetyl-CoA or longer chain acyl-CoAs, respectively. The thioester could undergo enzyme-catalyzed hydrolysis. Alternatively, spontaneous thioester hydrolysis is known to be an important step in the mammalian blood clotting cascade (Fredslund et al. 2006. J. Mol. Biol. 361: 115-127). Thioester hydrolysis and facile β-ketoacid decarboxylation offers a plausible explanation as to why monoketones have been observed whenever the oleA gene, by itself, is cloned into a heterologous host Weller et al. Appl. Environ. Micro. 76:1212-1223; Friedman et al. 2008. International Patent Application WO 2008/147781; Friedman et al. 2008. International Patent Application WO 2008/113041). Moreover, ketones were observed in this study when exogenous oleA genes were placed into the S. oneidensis MR-1 background.

III. Purification and Characterization of OleA from Xanthomonas campestris and Demonstration of a Non-decarboxylative Claisen Condensation Reaction

OleA catalyzes the condensation of fatty acyl groups in the first step of bacterial long-chain olefin biosynthesis but the mechanism of the condensation reaction is controversial. In this study, OleA from Xanthomonas campestris was produced in Escherichia coli and, purified to homogeneity. The purified protein was shown to be active with fatty acyl-CoA substrates that ranged from C₈ to C₁₆ in length. With limiting myristoyl-CoA (C₁₄), one mole of the free coenzyme A was released per mole of myristoyl-CoA consumed. Using [¹⁴C]-myristoyl-CoA, the other products were identified as myristic acid, 2-myristoylmyristic acid and 14-heptacosanone. 2-Myristoylmyristic acid was indicated to be the physiologically-relevant product of OleA in several ways. First, 2-myristoylmyristic acid was the major condensed product in short incubations but, over time, it decreased with the concomitant increase of 14-heptacosanone. Second, synthetic 2-myristoylmyristic acid showed similar decarboxylation kinetics in the absence of OleA. Third, 2-myristoylmyristic acid was shown to be reactive with purified OleC and OleD to generate the olefin 14-heptacosene, a product seen in previous in vivo studies. The decarboxylation product, 14-heptacosanone, did not react with OleC and OleD to produce any demonstrable product. Substantial hydrolysis of fatty acyl-CoA substrates to the corresponding fatty acids was observed but it is currently unclear if this occurs in vivo. In total, these data are consistent with OleA catalyzing a non-decarboxylative Claisen condensation reaction in the first step of the olefin biosynthetic pathway previously found to be present in at least 70 different bacterial strains.

### III-A. Experimental Procedures

Chemical Synthesis and analysis. β-Ketocarboxylic acid syntheses have been previously reported (Detalle et al. 2004. JOC. 69:6528-6532), but the literature does not describe the synthesis of higher benzyl esters of β-ketocarboxylic acids derived from fatty acids. The detailed procedure used for the synthesis of 2-myristoylmyristic acid (2MMA, 2-dodecyl-3-ketohexadecanoic acid) is described below.

In brief, the forced Claisen condensation method described by Briese and McElvain (Briese et al. 1933. J. Am. Chem. Soc. 55:1697-1700; Adams et al. 1942. Organic Reactions, vol. 1, 9^{th} Ed. John Wiley & Sons, Inc. New York) was adapted to the coupling of benzyl myristate, using a half equivalent of sodium benzyl alcoholate in benzyl alcohol as the basic promoter, removing benzyl alcohol by heating under vacuum to force the condensation nearly to completion. Benzyl 2-myristoyl-myristate (B2MM, benzyl 2-dodecyl-3-ketohexadecanoate) so prepared (70% yield and approximately 90% purity after bulb-to-bulb distillation) crystallized, mp 34.5-35.8° C. The purity of B2MM was estimated from NMR data in CDCl₃ solution, which show no conclusive evidence for tautomeric enolic forms being present.

Carefully monitored hydrogenolysis of B2MM (Pd/C catalyst, methyl t-butyl ether solvent) isolated by filtration and cooling of the filtrate to −80° C., produced a 5:2 (mol/mol) mixture of 2MMA and its decarboxylated product 14-heptacosanone as determined by GC-MS analysis after methylation of the acid (CH₂N₂). On cold storage (−80° C.) the solution was enriched to an 8:1 2MMA and the derived ketone, apparently by preferential precipitation of the ketone.

Cloning and Production of OleA. Synthetic oleA genes were designed based on oleA genes from Congregibacter litoralis KT71 (ZP_{—}01103251.1), Xanthomonas campestris spv. campestris str. ATCC 33913 (NP_{—}635607.1), Xylella fastidiosa 9a5c (NP_{—}299252.1), Plesiocystis pacifica SIR-1(ZP_{—}01906524.1), and γ-proteobacterium NOR5-3 (ZP_{—}05127044.1), see Supplementary FIG. III-1S, and purchased from DNA 2.0 (Menlo Park, Calif. The genes were cut with NdeI and BamHI restriction enzymes and cloned into pET28b+(Novagen, Madison, Wis.). All five genes were separately transformed into E. coli One Shot BL21 (DE3) (Invitrogen). All five recombinant strains were screened for soluble protein produceion in 50 ml cultures induced for 4 h at 37° C. Two of the five constructs produced soluble protein in E. coli, only X. campestris was found to be active in vitro, and that was selected for further study.

X. campestris for OleA purification was cultivated under two different conditions. Small-scale cultivations were conducted in 2 L flasks containing 500 ml LB with 50 μg/ml kanamycin and induced at an OD₆₀₀ of 0.7-0.85 with 0.1 M isopropyl-β-D-thiogalactopyranoside (IPTG). After 4 h, cells were harvested by centrifugation for 25 min at 3000 g. Large-scale cell cultivation was conducted in the Biotechnology Resource Center, University of Minnesota. A 440 L culture was prepared in a 550 L DCI bioreactor (DCI-Biolafitte, St. Cloud, Minn.) using a Rhapsody digital controller system and induced with 0.5 mM IPTG. Cells were harvested, lyophilized, and then stored at −80° C.

Purification of OleA. Cells were resuspended in 20 mM sodium phosphate buffer, 500 mM NaCl pH 7.4 with EDTA-free protease inhibitor tablets (Roche). Cells were disrupted by 3 passes through a chilled French pressure cell at 1200 psi and centrifuged at 27,000 g for 90 min to obtain the soluble protein fraction. The soluble fraction was centrifuged at 27,000 g for 30 min to clear prior to loading onto a Pharmacia Biotech LCC 501 FPLC equipped with a 5 ml Ni(II)-loaded HisTrap HP column (Amersham Biosciences) equilibrated with 20 mM sodium phosphate, 500 mM NaCl pH 7.4 buffer. The OleA protein was eluted at 135 mM imidazole. Ten g wet weight of cell paste yielded 60 mg purified OleA. Fractions were analyzed by SDS-PAGE and Simply Blue Safestain (Invitrogen). Pooled fractions were concentrated, and imidazole removed, with 3 passes through a 50 ml pressure concentrator (Amicon) using a 10,000 MWCO membrane (Millipore). Alternatively, after concentration of fractions, the OleA protein was dialyzed 3 times at 4° C. to remove the imidazole. Protein concentrated up to 30 mg/ml remained soluble and active.

Identification and Produceion of Active OleD. Using the NCBI Blast algorithm (Altschul et al. 1997. Nucleic Acids Res. 25:3389-3402), oleD genes were identified. Sequences from Chloroflexus auranticus (Caur_{—}3530), γ-proteobacterium NOR5-3 (ZP_{—}05127041.1), Xylella fastidiosa Temecula1 (NP_{—}779252.1), Xanthomonas campestris spv. campestris str. ATCC 33913 (NP_{—}635614.1) were optimized for produceion in E. coli (see Supplementary FIG. III-2S) and cloned into pJproduce produceion vectors with a T7 promoter by DNA 2.0 (Menlo Park, Calif.). Vectors were transformed into E. coli One Shot BL21 (DE3) (Invitrogen). Proteins were screened for activity and produceion using 50 ml LB cultures with 50 μg/ml kanamycin. Cells were induced with 0.1 mM IPTG at an OD₆₀₀ of 0.55-0.75. Soluble cell extracts were combined with OleA, OleC, and cofactors to test for the production of alkenes using the GC-MS enzyme assay described below. The OleD protein originating from X. campestris was the only protein found to support alkene biosynthesis. Cultures were scaled up using 2 L flasks containing 500 ml LB with 50 μg/ml kanamycin. Cultures were grown at 37° C. with agitation at 225 rpm. The culture was induced at an OD₆₀₀ of 0.7-0.8 with 0.1-0.4 mM IPTG and grown at 30° C. shaking at 225 rpm for 20 h. Cells were harvested by centrifugation for 25 mM at 3000 g.

Purification of OleC, OleD, and Assay of OleD. The cloning, produceion, and purification of OleC was previously described (Frias et al. 2010. Acta Crystallogr F. 66:1108-1110). For purification of OleD, cell pellets were resuspended in 20 mM sodium phosphate, 500 mM NaCl, pH 7.4 with EDTA-free protease inhibitor tablets (Roche) and passed through a chilled French pressure cell three times at 1200 psi. The cell lysate was centrifuged at 27,000 g for 90 mM and the soluble fraction was centrifuged for an additional 30 min. The soluble fraction was passed though a 0.20-μm syringe filter prior to chromatography. A 5 ml Ni(II)-loaded HisTrap HP column (Amersham Biosciences) equilibrated with 20 mM sodium phosphate, 500 mM NaCl pH 7.4 buffer was used for purification. Alternatively, 50 mM MOPS, 1% Tween 20, pH 7.0 was used for purification to improve solubility. Fractions were analyzed for purity by SDS-PAGE and Simply Blue Safestain (Invitrogen). Fractions eluting at 450 and 500 mM imidazole were pooled. The pooled protein was concentrated using a 50 ml pressure concentrator (Amicon) with a 10,000 MWCO membrane (Millipore) and dialyzed three times at 4° C. After dialysis, protein was centrifuged at 14,000 g for 15 min at 4° C. to remove precipitated protein.

OleD was previously suggested to be a ketone reductase (Sukovich et al. 2010. Appl. Environ. Microbiol. 76:3850-62). It was shown here to be active in a 250 μl reaction mixture consisting of 100 mM Tris, pH 7.4 containing OleA, OleD, OleC, 8 mM MgCl₂, 80 μM ATP, 260 μM myristoyl-CoA and 120 μM NADPH. NADPH oxidation was followed spectrophotometrically at 340 nm.

Detecting the Release of CoASH Thiol for Assay of OleA Substrate Range. Release of the free thiol group of CoASH was detected by the addition of 5,5′-dithio-bis-(2-nitrobenzoic acid) (DTNB) measured spectrophotometrically at 412 nm (ε₄₁₂=13,600 M⁻¹ cm⁻¹) (Alexson et al. 1988, J. Biol. Chem. 263:13564-13571; Ellman et al. 1958. Arch Biochem. Biophys. 74:443-450). Acyl-CoA substrates, purchased from Sigma Aldrich (Milwaukee, Wis.), were reacted with OleA protein in 100 mM Tris pH 7.4 and incubated at room temperature for 5 min in either 1 ml or 250 μl. DTNB was incubated with the reaction for 2 min and quantified spectrophotometrically.

Hydrocarbon Detection Enzyme Assay. A glass vial containing 250 μl total volume of 100 mM Tris pH 7.4 with 200-600 μg OleA, 5-25 μg OleD, 66 μg OleC, 1.4 mM NADPH, 8 mM MgCl₂, 3 mM ATP, and 1.2 mM myristoyl-CoA or an excess of 14-heptacosanone were incubated overnight at 30° C. with gentle shaking. Products were extracted with 250 μl ethyl acetate using 16-hentriacontanone ketone (Tokyo Kasei Kogyo Co., Ltd., Japan) as an internal standard. After vortexing and 5 min of gentle centrifugation, the top solvent layer was transferred to a glass vial and analyzed using a gas chromatograph equipped with a flame ionization detector HP 7890A (Hewlett Packard, Palo Alto) and mass spectrometer HP 5975C (GC-MS-FID). GC was conducted under the following conditions: helium gas, 1.75 ml/min; HP-1 ms column (100% dimethylsiloxane capillary; 30 m by 250 μm by 0.25 μm); temperature ramp, 100° C. to 320° C.; 10° C./min, hold at 320° C. for 5 min, 250° C. injection port, and split at the outlet between MS and FID. The mass spectrometer was run under the following conditions: electron impact at 70 eV and 35 μA. The flame ionization detector was set at 250° C. with hydrogen flow set at 30 ml/min, air set at 400 ml/min, and helium makeup gas set at 25 ml/min.

GC-MS was also used as described (Frias et al. 2009. Appl. Environ. Microbiol. 75:1774-1777) for detecting ketones derived from spontaneous decarboxylation of the OleA β-keto acid products, using 200-600 μg OleA and 1 mM acyl-CoA substrates.

Radiolabeled Acyl-CoA Assay. Reactions of 200 μl included 0.2 μCi [1-¹⁴C]-myristoyl-CoA, 40-60 mCi/mmol (American Radiolabelled Chemical, St. Louis, Mo.), 750 μM myristoyl-CoA and 1 mg OleA in 100 mM Tris pH 7.4. Samples were analyzed using high pressure liquid chromatography (HPLC) on a Shimadzu HPLC system equipped with a UV detector (Shimadzu, Columbia, Md.) and a β-ram radioflow detector operated with the Laura 4 data acquisition/evaluation software (IN/US Systems, Tampa, Fla.). UV detection was set at 259 or 274 nm. Unfiltered samples of 50 or 100 μl volume were injected onto an analytical reverse phase Alltima HPC8 column with 5 μm packing (Alltech 250×4.6 mm) and C8 guard column. The column was equilibrated in 50% 20 mM ammonium acetate pH 5.4 (A) and 50% 85:15 acetonitrile:methanol (B) and the following method adapted from (22). Linear gradients were as follows: 50% A:50% B 0-10 min, ramp to 70% B 10-15 min, 70% B 15-30 min, ramp to 100% B 30-35 min, 100% B 35-50 min, return to 50% A:50% B 50-55 min and equilibrate 55-70 min. The flow rate was 1 ml/min. The scintillant (Monoflow X; National Diagnostics, Atlanta, Ga.) flow rate was 3 ml/min.

Mass Spectrometry Analysis. Mass spectrometry on enzymatically-produced and synthetic β-keto acid was performed using an LCQ-classic (Thermo Fisher Scientific) ion trap mass spectrometer with electrospray ionization mode (ESI). Samples were introduced by loop injection of 5 μl. Product ion spectra for 2-myristoylmyristic acid m/z 437 (M-H) was identified in negative ion mode, as well two additional ions m/z 393 and m/z 473/475. The fatty acid HPLC peak was analyzed by direct infusion into a Quantum Discovery Max (Thermo Finnigan) mass spectrometer operated in negative ion mode. ESI⁻-MS spectra for myristic acid was m/z 227 (M-H). Electron impact mass spectrometry in conjunction with GC (GC-MS) was performed to obtain the spectra of the β-keto acid derivatized with diazomethane. The solvent was evaporated with N₂, and then taken up in methyl-t-butyl-ether (mtbe) to run on GC-MS as previously described (Frias et al. 2009. Appl. Environ. Microbiol. 75:1774-1777). The ketone molecular ion, m/z 394, was also identified by this method.

Analytical Gel Filtration. A Superdex 75 10/100 GL (Amersham Biosciences) size exclusion column was used on an AKTA (General Electric) FPLC with elution at 0.5 ml/min. The column was equilibrated with 20 mM sodium phosphate, 500 mM NaCl, pH 7.4. Molecular weight standards (Biorad, Hercules, Calif.) were used with a range of 1,350-670,000 to create a standard curve. Three additional standards were used in a closer MW range to where OleA eluted, chymotrypsin (Mᵣ=25 kDa), albumin (Mᵣ=67 kDa) and conalbumin (Mᵣ=77 kDa).

Detailed Methods and Scheme of Chemical Synthesis of 2-Myristoyl Myristic Acid (2MMA) and Analysis.

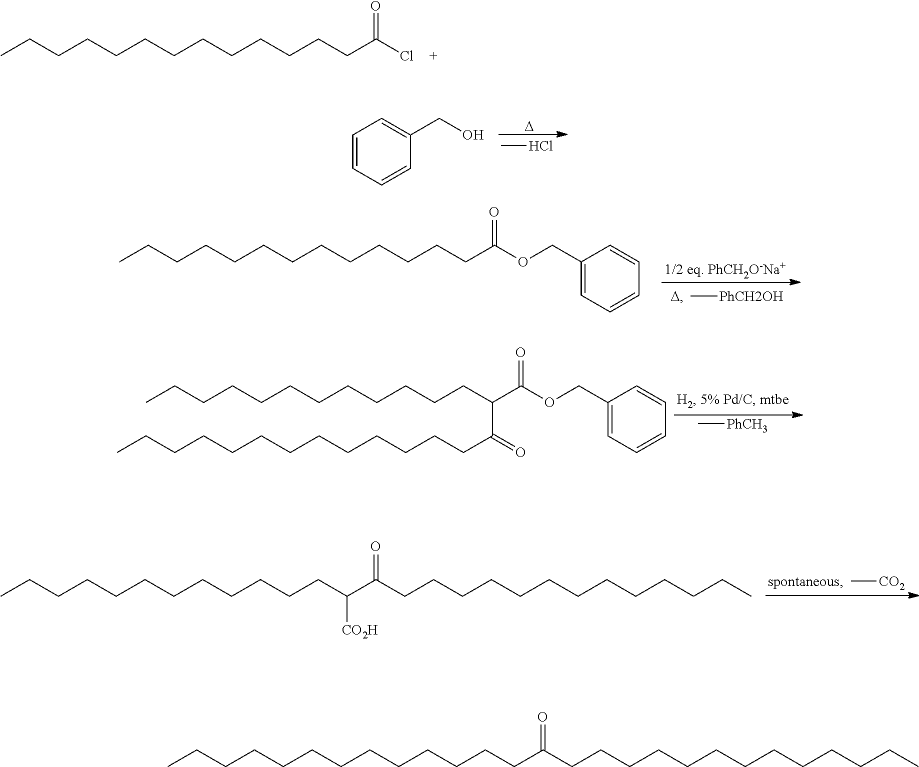

General. ¹H and ¹³C NMR were obtained at 400 and 100 MHz, respectively.

Benzyl myristate. Benzyl myristate was prepared essentially by the method of Shonle and Row (Shonle et al. 1921. J. Am. Chem. Soc. 43:361-5) by the neat (solventless) reaction of myristoyl chloride and slight excess benzyl alcohol. After evacuation to remove volatiles, NMR analysis indicated that no myristoyl chloride and only benzyl myristate, the excess benzyl alcohol and a trace of benzyl chloride remained. This product was used without further purification.

Benzyl 2-Myristoylmyristate (B2MM). A distilling flask was charged with 6 mL of benzyl alcohol and 1 mL of this was distilled (97° C./20 mmHg) to dry the residue and remove volatiles. Sodium hydride powder (0.12 g, 5 μmol, pentane washed oil dispersion, filtered and N₂ dried) added to the cooled dry benzyl alcohol under N₂ evolved hydrogen but fully dissolved only after warming and stirring. The sodium benzyl alkoxide/benzyl alcohol solution was added under N₂ to a reaction flask charged with 3.2 g (10 μmol) of benzyl myristate, a stir bar and steam-heated (100° C.) reflux condenser with vacuum takeoff to a cold trap. The reaction mixture was evacuated (0.1 mmHg) and slowly heated to remove excess benzyl alcohol. Finally, the reaction was heated over two hours from 120 to 132° C./0.1 mmHg leaving a semi-solid residue. Neutralization (HOAc) and NMR analysis indicated that the reaction had proceeded to about 70% conversion. Bulb-to-bulb distillation of 90% of the crude product mixture to 150° C./0.05 mmHg left 1.35 g (ca. 70% yield) of reasonably pure B2MM, which slowly crystallized to a waxy solid (mp 34.5-35.8° C.). ¹H NMR integrations of the aryl and high-field signals are in 10% excess of theory, suggesting that this product is approximately 90% pure B2MM. There is no evidence for tautomeric (enolic) content, which may account in part for the integral disparities.

¹H NMR (CDCl₃): 7.3-7.4 (m, 5H, aryl), 5.17 and 5.14 (prochiral benzylic AB, J=12.3 Hz, 2H), 3.46 (t, 1H, CH, J=7.4), 2.46 and 2.40 (each dt, 2H, prochiral CH₂, J=17.2 and 7.2 Hz), 1.84 (m, 2H, α-CH₂), 1.52 (ft, 2H, 13-CH₂), 1.34-1.16 (m, 10H, CH₂) and 0.88 ppm (t, 6H, CH₃).

¹³C NMR (CDCl₃, assigned as 77.0 ppm): 205.1, 169.7, 135.4, 128.5, 128.3, 128.2, 66.8, 59.1, 41.8, 31.9, 29.63, 29.60, 29.55, 29.45, 29.4, 29.30, 29.28, 29.26, 28.9, 28.2, 27.4, 23.4, 22.6 and 14.0 ppm. (Twenty four of 33 possible carbon singlets are resolved.)

Hydrogenolysis of Benzyl 2-Myristoylmyristate: 2-Dodecyl-3-ketohexadecanoic Acid (2-Myristoylmyristic Acid, 2MMA) and Methyl 2-Dodecyl-3-ketohexadecanoate (Methyl 2-Myristoylmyristate, M2MM). A shielded apparatus with gas inlet (top) and outlet that could be positioned to near the bottom (for complete gas purging) was charged with 17 mg B2MM, 3.4 mg 5% Pd/C catalyst suspended in 3 mL methyl t-butyl ether (mtbe). The apparatus was thoroughly purged with N₂, then with H₂ (industrial grade) and maintained under H₂ at atmospheric pressure (Caution: all oxygen must be removed before hydrogen is introduced: H₂/O₂ or air within the explosive limits plus catalyst will detonate). After 2.5 h, stirring was interrupted, and the settled mixture was sampled. The sample was immediately treated (shield) with slight excess of ethereal diazomethane (yellow persisted) and concentrated. Analyses by NMR and GC showed that the hydrogenolysis was 97% completed. After stirring an additional hour, the reaction mixture (N₂ purged) was filtered and the filtrate and washes were immediately cooled (dry ice), and stored in −80° C. freezer. A sample of this product, warmed to −18° C., treated with diazomethane, and analyzed by GC, showed a 5:2 mixture of M2MM and the product of decarboxylation of the β-keto acid, 14-heptacosanone, plus only minor impurities. After two weeks storage at −80° C., GC analyses showed that the supernate had enriched to 8:1 β-keto acid (→M2MM) and ketone. This indicates that partial crystallization of the ketone had occurred.

NMR of M2MM (CDCl₃): essentially identical patterns (slightly different shifts) to B2MM absent benzyl signals plus methyl ester singlet at 3.72 ppm.

¹H NMR of 14-heptacosanone (CDCl₃): 2.38 (t, 4H, α-CH₂, J=7.4), 1.56 (m, 4H, β-CH₂), 1.32-1.22 (m, 40H), 0.88 (t, 6H, CH₃).

### III-B. Results

Cloning and Produceion of oleA Genes, and Purification of OleA Protein. The oleA genes from Congregibacter litoralis KT71, Xanthomonas campestris spv. campestris str. ATCC 33913, Xylella fastidiosa 9a5c, Plesiocystis pacifica SIR-1, and γ-proteobacterium NOR5-3 were each cloned into E. coli and tested for the production of soluble OleA protein. The recombinant E. coli containing the X. campestris gene showed a high amount of soluble OleA protein as determined by SDS-PAGE and was found to be active in purified form, and that strain was therefore selected for further studies. E. coli cells producing a His-tagged X. campestris OleA protein were grown in a 550 L bioreactor vessel, harvested, and lysed. Following chromatography on a Ni-column, the protein was shown to be homogenous as indicated by SDS-PAGE (FIG. III-2B).

General Characteristics of OleA. The subunit molecular weight of the native OleA protein is 36,629 but, as engineered here with the His-tag, it is 38,792. The protein migrates somewhat higher than this on SDS-PAGE (FIG. III-2B). The OleA protein subunit MW is near the middle of the range found in homologous proteins from the thiolase superfamily (Table III-1). The Mycobacterium Pks13 protein is a large multidomain protein with the condensing enzyme domain defined as 44,122 in the annotation on the NCBI server. The native molecular weight of OleA was estimated to be 62,000 by gel filtration chromatography. This is suggestive of a subunit stoichiometry of two for the native enzyme. Hydroxymethylglutaryl-CoA (HMG-CoA) reductase, FabH and the Mycobacterium Pks13 are all dimers and the Zoogloea thiolase is a tetramer (Davis et al. 1987. J. Biol. Chem. 262:82-89; Gavalda et al. 2009. J. Biol. Chem. 284:19255-19264; Davies et al. 2000. Structure. 8:185-195; Clinkenbeard et al. 1975. J. Biol. Chem. 250:3124-3135).

The OleA protein shows a low sequence relatedness with homologous proteins in the thiolase superfamily (Table III-1). With such divergence, it is not surprising that the cellular functions of the proteins are quite different. However, the general biochemical reaction catalyzed by all of the enzymes shown in Table III-1 involves the condensation of acyl substrates. These condensation reactions occur by either a decarboxylative or non-decarboxylative (Heath et al. 2002. Nat. Prod. Rep. 19:581-596) mechanism and the results described below are consistent with a non-decarboxylative mechanism for OleA. In both cases, thiolase superfamily proteins use a conserved active site cysteine and generate an acyl enzyme intermediate (Haapalainen et al. 2006. TRENDS Biochem. Sci. 31:64-71). OleA shares this conserved active site cysteine residue (Table III-1). In the vicinity of the conserved cysteine, the amino acids in the OleA from X. campestris and M. luteus (Beller et al. 2010. Appl. Environ. Microbiol. 76:1212-1223) are highly conserved (Table III-1).

TABLE III-1
Properties of OleA compared with homologous proteins in the thiolase
superfamily.

| | | % | | | | | |
|---|---|---|---|---|---|---|---|
| Protein | | Seq | Calc | Calc | Cellular | Claisen | Sequence |
| (organism) | Accession # | ID* | MW | pI | Function | Mechanism | Signature^{#} |
| OleA (X. | NP_635607 | 100 | 36,629 | 5.6 | Alkene | Proposed | NACLAFING |
| campestris) | | | | | bio- | Non- | |
| | | | | | synthesis | Decarboxylative | |
| OleA (M. | YP_00295738 | 38 | 36,653 | 4.8 | Alkene | Proposed | NACLGFVNG |
| luteus)^{11@} | 2.1 | | | | bio- | Decarboxylative | |
| | | | | | synthesis | | |
| Thiolase | AAA27706.1 | 19 | 40,416 | 5.9 | PHB | Non- | QLCGSGLRA |
| (Z. ramigera)²³ | | | | | bio- | Decarboxylative | |
| | | | | | synthesis | | |
| HMG-CoA | 1XPL_A | 16 | 43,204 | 5.0 | Mevalonate | Non- | EACYAATPA |
| synthase (H. | | | | | pathway | Decarboxylative | |
| sapiens)²⁶ | | | | | | | |
| FabH | 1EBL_A | 24 | 33,523 | 5.1 | Fatty Acid | Decarboxylative | AACAGFTYA |
| (E. coli)²⁵ | | | | | bio- | | |
| | | | | | synthesis | | |
| Mycobacterium | CAA17864 | 19 | 44,122 | 5.2 | Mycolic | Decarboxylative | TACSSSLVA |
| Pks13⁺²⁴ | | | | | Acid | | |
| | | | | | bio- | | |
| | | | | | synthesis | | |

*Via Needleman-Wunch and BLAST algorithms and comparison to OleA from X. campestris
⁺Alignment to keto-acyl synthase domain only, as defined by NCBI
^{#}Amino acid sequence surrounding active site cysteine conserved in thiolase superfamily proteins
^{@}Number of the reference from which the data represented in the table was obtained

Initial Defining of Substrate Specificity and Reaction Products of OleA. Enzymes catalyzing condensation or hydrolysis reactions with acyl-CoA substrates release coenzyme A that can be assayed colorimetrically using DTNB (Alexson et al. 1988. J. Biol. Chem. 263:13564-13571; Skaff et al. 2010. Anal. Biochem. 396:288-296; Sleeman et al. 2004. J. Biol. Chem. 279:6730-6736; Yashiro et al. 1995. Biochim. Biophys. 1258:288-96). Both proposed mechanisms (FIG. III-1A&B) showed OleA-catalyzed coenzyme A release and this assay was used to monitor enzyme activity during purification. With purified OleA, DTNB was used to determine the stoichiometry of coenzyme A formation and to begin to discern the substrate specificity of OleA.

First, the coenzyme A product stoichiometry was determined using either myristoyl-CoA or palmitoyl-CoA and allowing the substrate to completely react. With either substrate, the reaction stoichiometry was 1.0 mole of coenzyme A released for each mole of acyl-CoA consumed. In this manner, acyl-CoA chains of different lengths were tested with OleA using a time of incubation in which palmitoyl-CoA reacts completely as described in the Methods section. Under those conditions (Table III-2) palmitoyl-CoA reacted more completely than myristoyl-CoA. Octanoyl-, decanoyl-, lauroyl-, palmitoleyl-, and stearoyl-CoA were also found to undergo reaction to release coenzyme A (Table III-2), but acetyl-CoA did not.

TABLE III-2
Substrate specificity of OleA as determined by CoA release. Values
shown are the average of triplicate determinations with standard error.

| Substrate | | CoA | % of |
|---|---|---|---|
| Common Name | Carbon Chain Length | Product (μM)¹ | Theoretical² |
| Palmitoyl-CoA | 16 | 65.0 +/− 0.9 | 100 |
| Myristoyl-CoA | 14 | 63.2 +/− 0.4 | 97 |
| Lauroyl-CoA | 12 | 51.4 +/− 1.9 | 79 |
| Palmitoleoyl-CoA | 16 | 36.9 +/− 0.9 | 57 |
| Decanoyl-CoA | 10 | 27.2 +/− 1.6 | 42 |
| Stearoyl-CoA | 18 | 18.7 +/− 1.8 | 29 |
| Octanoyl-CoA | 8 | 8.0 +/− 2.2 | 12 |
| Acetyl-CoA | 2 | ND³ | ND |

¹Free coenzyme A detected as described in the methods
²Starting substrate was 65 μM; 65 μM of product is 100% of theoretical yield
³ND = No detectable activity

Subsequent experiments were conducted to examine if coenzyme A was formed as a consequence of acyl-group condensation, thioester bond hydrolysis, or some mixture of the two reactions. Based on previous observations (Beller et al. 2010. Appl. Environ. Microbiol. 76:1212-1223; Sukovich et al. 2010. Appl. Environ. Microbiol. 76:3850-62; Friedman et al. 2008. International Patent WO2008/113041), it was known that long-chain ketones were the observed condensation products. In subsequent experiments in this study, it was shown that β-keto acids are the initial products and those decarboxylate quantitatively to the corresponding ketone. In this context, reaction mixtures were solvent extracted and subjected to GC-MS to identify ketones derived from condensation and/or fatty acids derived from acyl chain hydrolysis.

Previous in vivo experiments identified asymmetric ketones, indicating that fatty acyl chains of different chain lengths could be condensed (Sukovich et al. 2010. Appl. Environ. Microbiol. 76:3850-62). In this context, experiments were conducted with mixtures of fatty acyl-CoA substrates. All pairwise combinations of C₁₀, C₁₂, C₁₄, C₁₆, saturated and C16 monounsaturated (C_{16:1}) acyl-CoA substrates were incubated, extracted, and analyzed for products by GC-MS and GC-FID. In all, 15 product mixtures were analyzed. The results are shown in Table III-3. It was found that acyl-CoA hydrolysis to the corresponding fatty acid was a major reaction in most cases. Only with C₁₂ acyl condensation and C₁₄ plus C_{16:1} condensation were the major products derived from a condensation of fatty acyl chains. In the case of C₁₄ condensations (myristoyl-CoA), the ketone 14-heptacosanone was produced at only slightly lower levels than the hydrolysis product myristic acid.

TABLE III-3
Product ratios determined by GC-MS for reactions of OleA with acyl-
CoA substrates of different carbon chain lengths as indicated by the left-hand column
and the top-row. The products, ketones (Cₓ) and fatty acids (FAₓ), are indicated in
order of decreasing abundance as determined by peak area integration as described in
the Methods section. The observed partitioning between condensation of similar or
different chains, or acyl-CoA hydrolysis, is illustrated at the bottom.

| Fatty acyl- | | | | | |
|---|---|---|---|---|---|
| CoA chains | C₁₀ | C₁₂ | C₁₄ | C₁₆ | C_{16:1} |
| C₁₀ | FA₁₀ > C₁₉ | FA₁₂ > FA₁₀ > | FA₁₀ > C₁₉ > | FA₁₆ > FA₁₀ > | FA₁₀ > FA_{16:1} > |
| | | C₂₃ > C₂₁ > C₁₉ | C₂₃ > C₂₇ > | C₁₉ > C₂₅ > | C_{25:1} > C₁₉ > |
| | | | FA₁₄ | C₃₁ | C_{31:2} |
| C₁₂ | — | C₂₃ | FA₁₄ > C₂₇ > C₂₅ > | FA₁₆ > C₂₃ > | FA_{16:1} > FA₁₂ > |
| | | | FA₁₂ > C₂₃ | FA₁₂ > C₂₇ > C₃₁ | C_{27:1} > C_{31:2} |
| C₁₄ | — | — | FA₁₄ > C₂₇ | FA₁₆ > C₂₇ > | C₂₇ > C_{29:1} > |
| | | | | FA₁₄ > C₂₉ > C₃₁ | FA₁₄ > C_{31:2} |
| C₁₆ | — | — | — | FA₁₆ > C₃₁ | FA_{16:1} > FA₁₆ > |
| | | | | | C_{31:2} > C_{31:1} |
| C_{16:1} | — | — | — | — | FA_{16:1} > C_{31:2} |

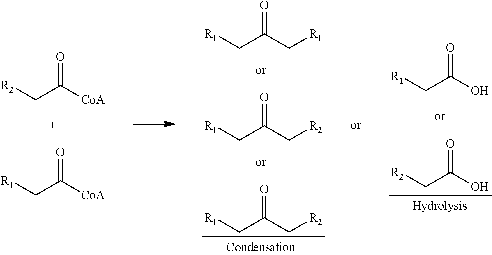

The identification of ketones as the condensation products by use of GC-MS led to the question of whether the decarboxylation was enzymatic or whether the decarboxylation occurs due to the labile nature of the reaction intermediate preceding the formation of the ketone. Further investigations were conducted using a C₁-labelled acyl-CoA substrate to track the carboxyl carbon.

Identification of Initial Condensation Product. OleA reactions with [1-¹⁴C]-myristoyl-CoA were analyzed using a high pressure liquid chromatograph (HPLC) fitted with a radioflow detector. A major peak eluting at 22.4 minutes was identified as myristic acid (FIG. III-3). The HPLC peak eluting at 44.3 (compound 2) min was analyzed by GC-MS and found to be 14-heptacosanone, but more of the radioactivity co-migrated with a more polar product eluting at 40.0 min (FIG. III-3). The major peak eluting at 40.0 min (compound 1) showed very little absorbance at 259 nm consistent with the absence of a coenzyme A moiety. Over time, the peak at 40 min diminished with a concomitant increase in the peak at 44.3 min (FIG. III-3, inset). This observation was consistent with a decarboxylation of the compound at 40.0 min giving rise to increasing concentrations of 14-heptacosanone over the course of 6.5 hours. This was also indicated because the compound eluting at 44.3 min contained only half of the ¹⁴C as did the compound at 40 min, consistent with a loss of a carbon atom as carbon dioxide. Moreover, β-keto acids are known to be labile and the decarboxylation of 2-myristoylmyristic acid would be expected to produce 14-heptacosanone

To investigate this further, the benzyl ester of 2-myristoylmyristic acid was synthesized. It was hydrogenolyzed with palladium and hydrogen to produce 2-myristoylmyristic acid. This latter compound was observed to undergo rapid decarboxylation to produce 14-heptacosanone.

Treatment of synthetic 2-myristoyl-myristic acid with diazomethane yielded the methyl ester. The synthetic methyl ester was compared to the enzyme-produced compound collected at 40.0 min that had been immediately reacted with diazomethane. Both methylated compounds showed a GC retention time of 20.6 min and essentially identical mass spectra (FIG. III-4). The parent ion at m/z 452 is present in both but it is a minor ion. In this context, electrospray ionization mass spectrometry was conducted on the free acid product 1 from the OleA reaction with myristoyl-CoA (Table III-4, top) and the synthetic standard 2-myristoylmyristic acid (Table III-4, bottom). In this case, a major negative ion was observed (m/z 437) with a mass of one less than the molecular mass of 2-myristoylmyristic acid in both the biological product and the standard. A second major fragment of m/z 393 found in both is consistent with the loss of carbon dioxide in the mass spectrometer. Another ion fragment was detected at m/z 473/475, suggested to be [M-H+HCl].

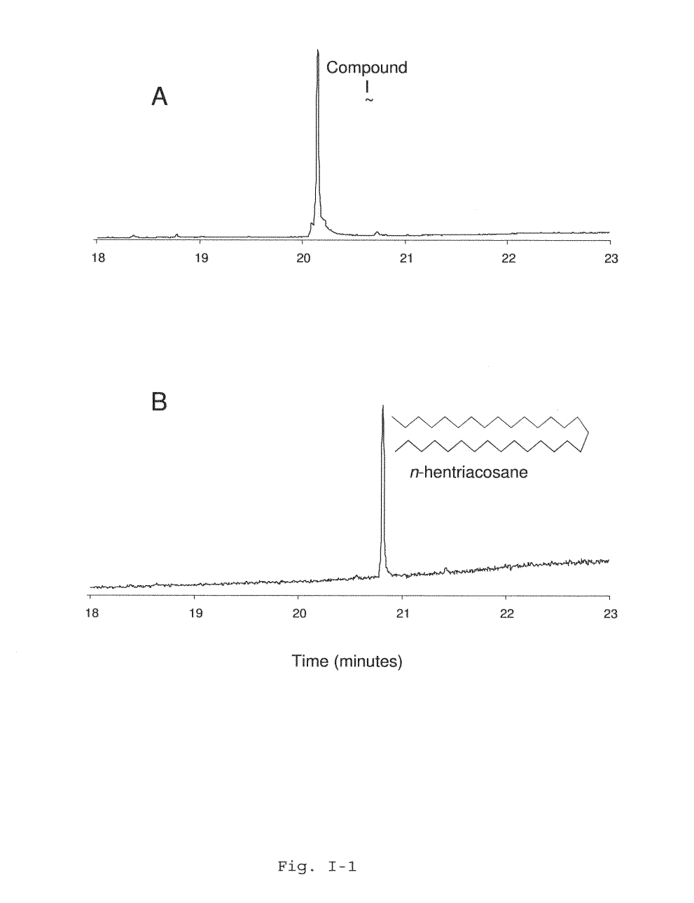

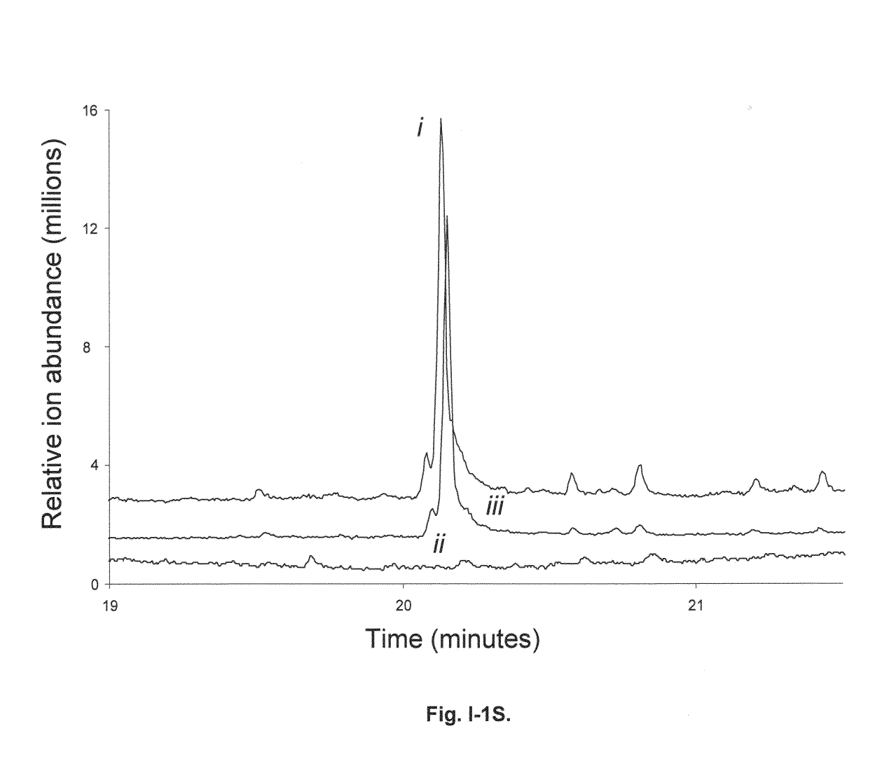

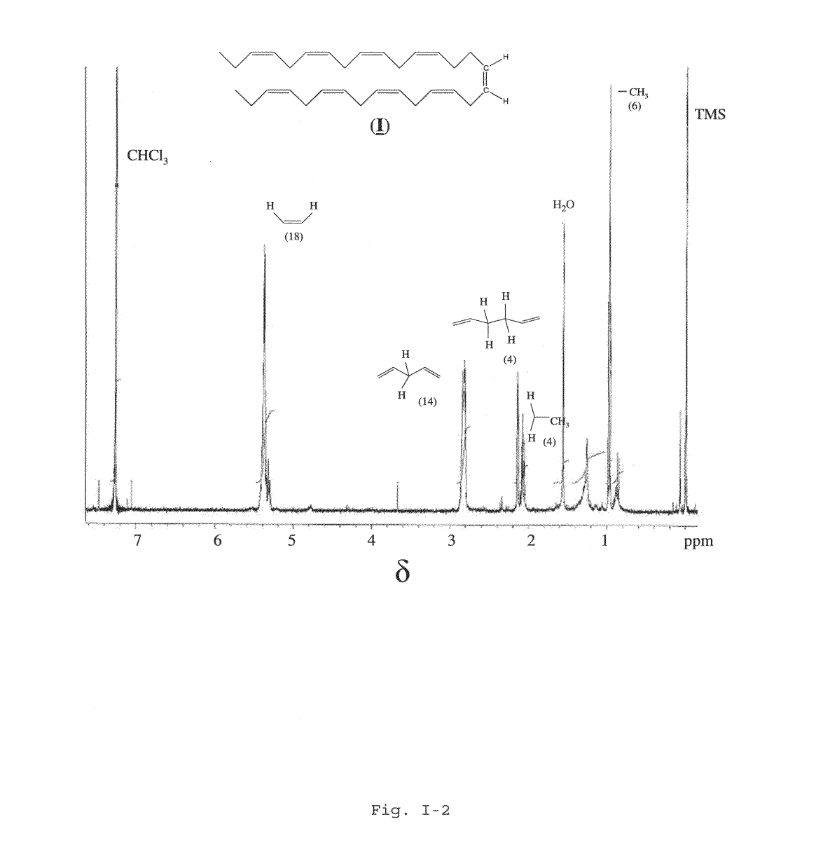

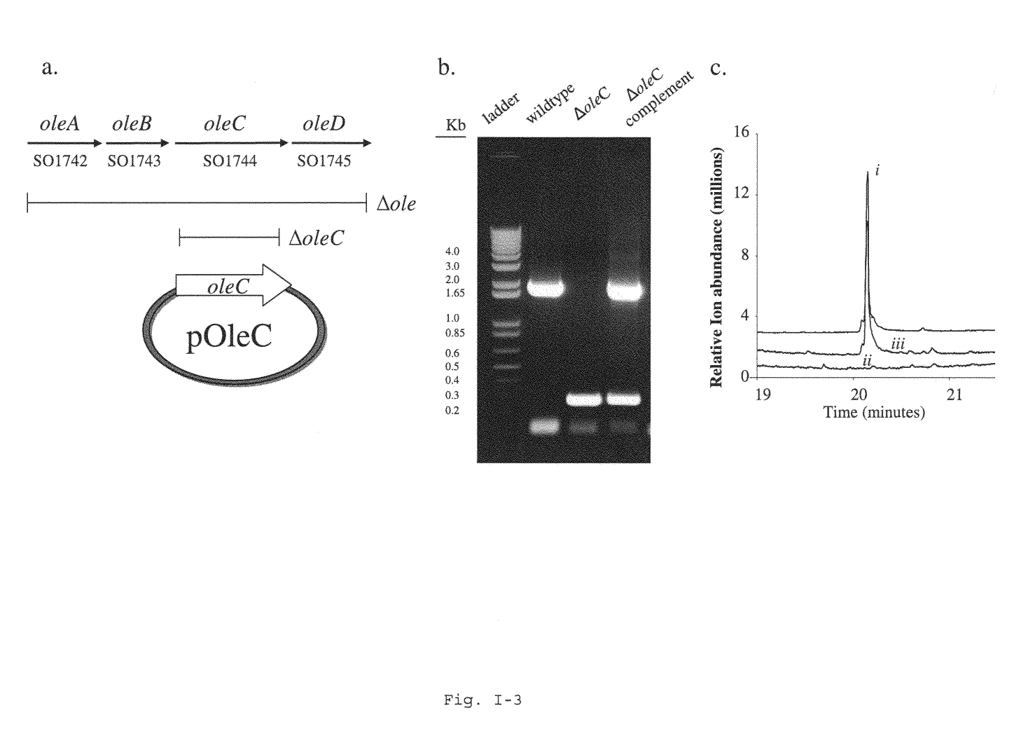

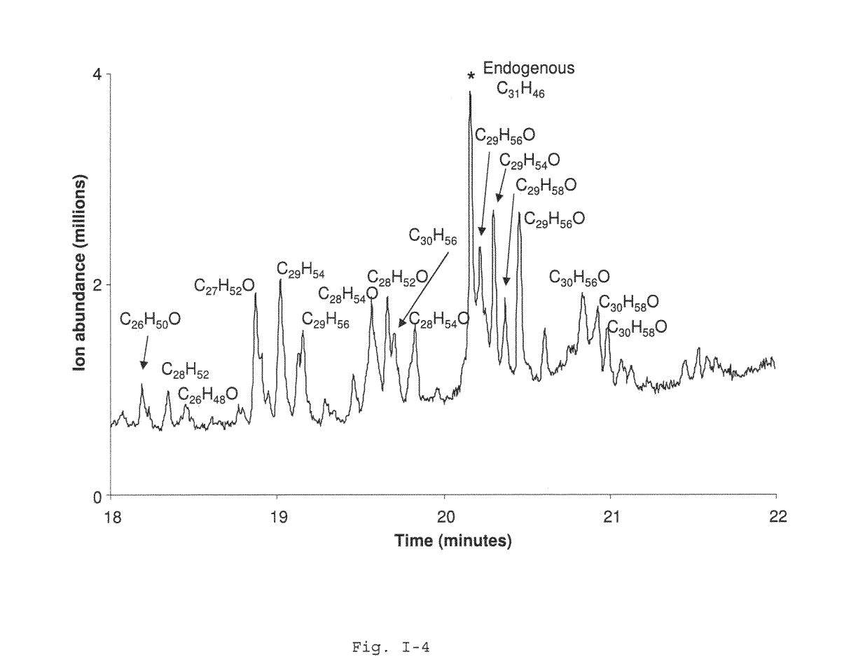

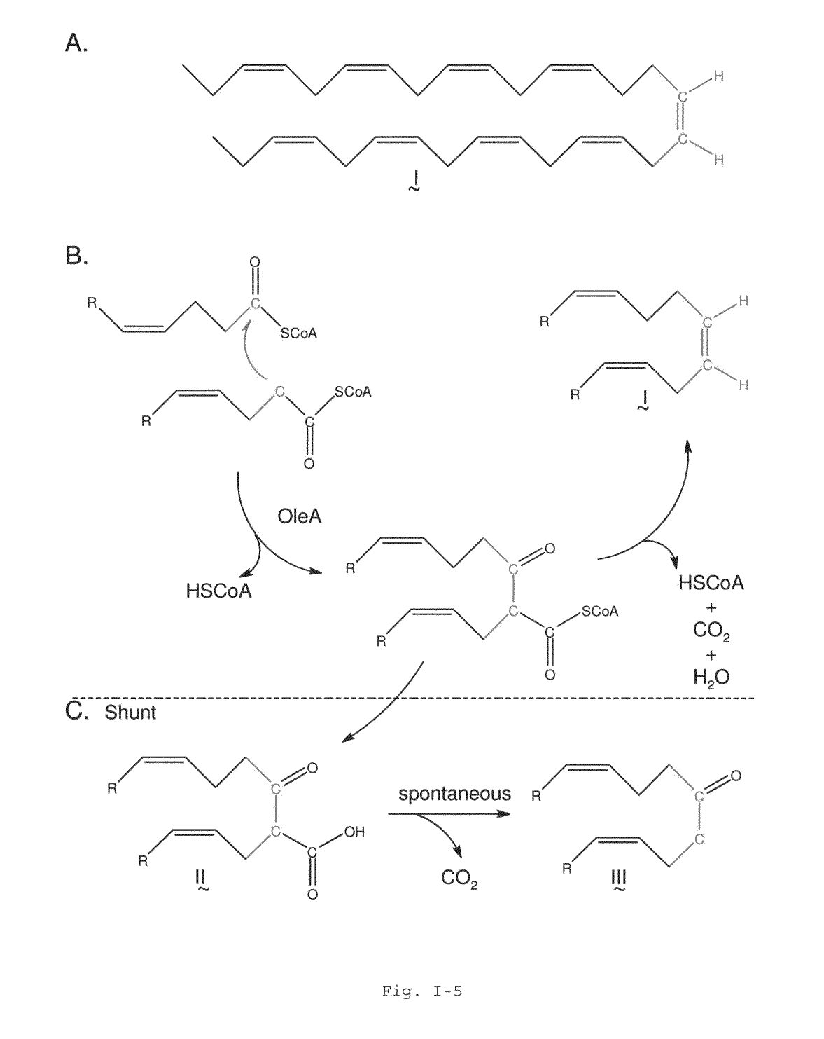

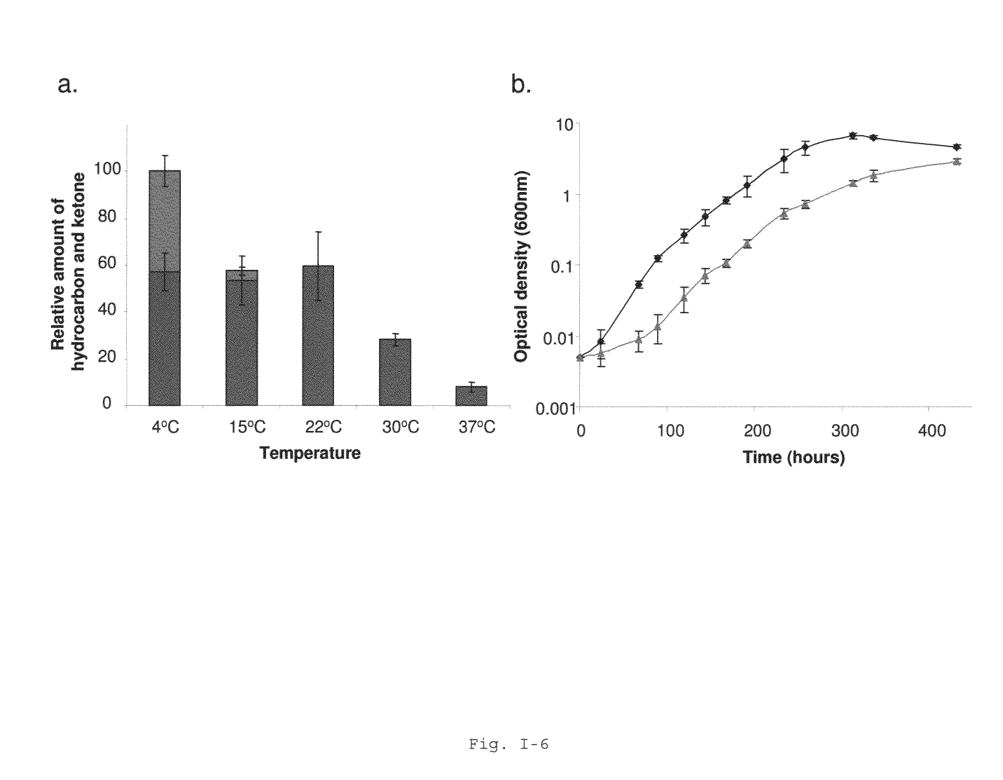

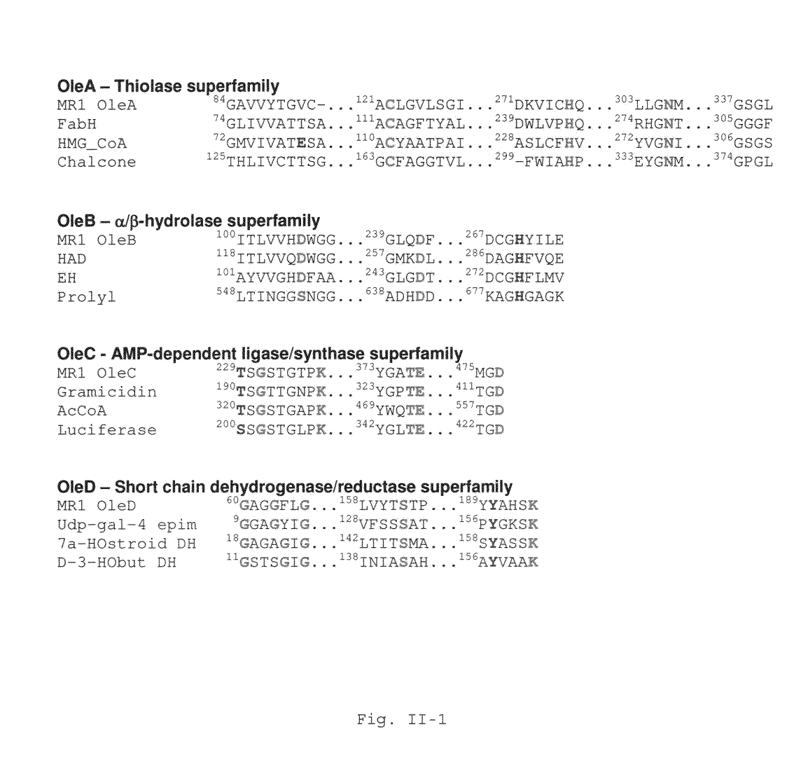

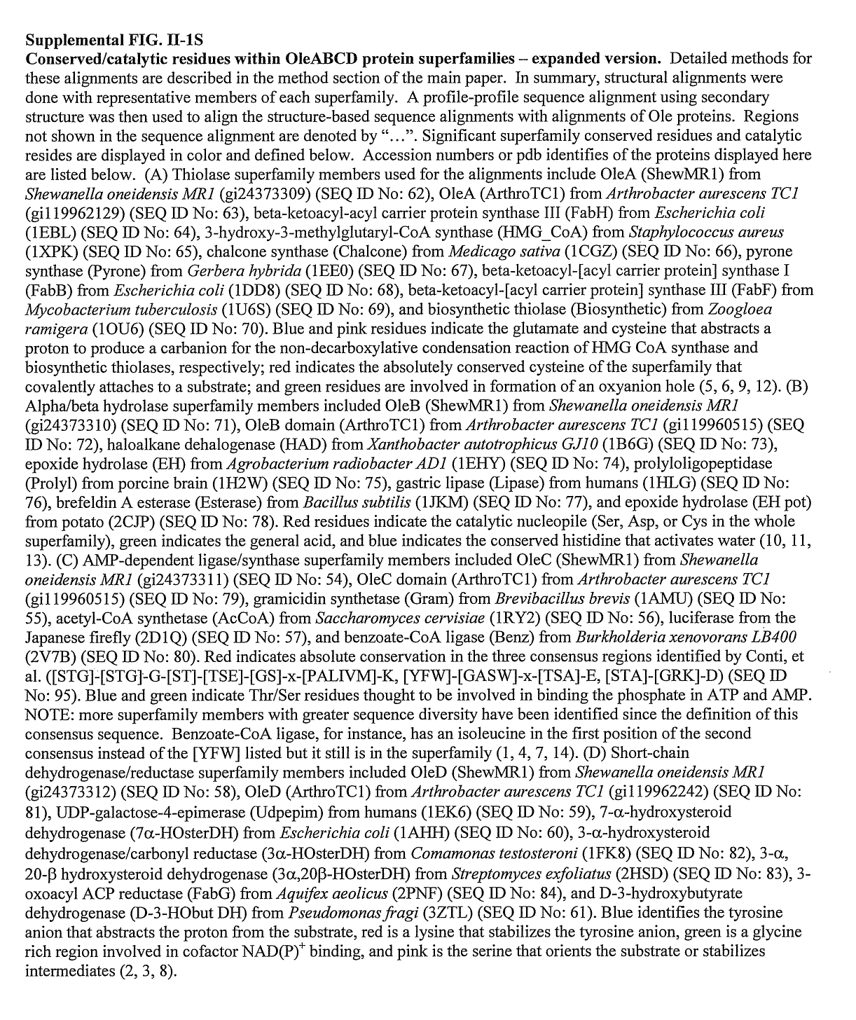

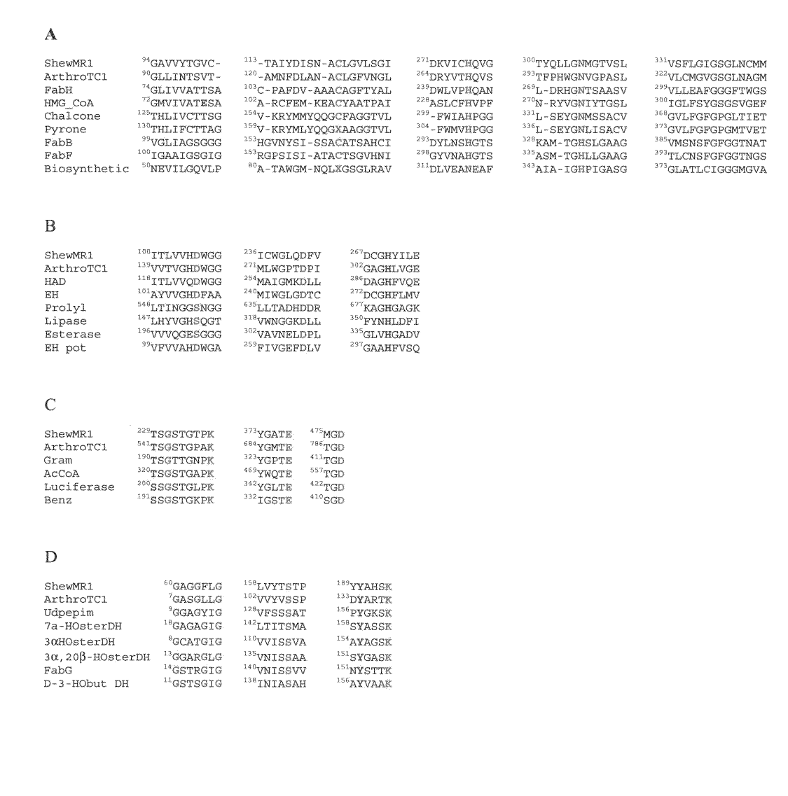

TABLE III-4
Electrospray-ionization (ESI) mass spectrometry of product 1 from
reaction of OleA with myristoyl-CoA as shown in FIG. 3 and
synthetic 2-myristoylmyristic acid.

| | Molecular | ESI, negative ion mode |
|---|---|---|
| Sample | Mass | m/z (intensity) |
| Product 1 | — | 393 (52), 437 (29), 473 (19) |
| 2-Myristoyl- | 438 | 393 (45), 437 (36), 473 (19) |
| myristic | | |
| acid | | |

Role of OleA in olefin biosynthesis. OleA has been proposed to function with other Ole proteins to produce olefins (Beller et al. 2010. Appl. Environ. Microbiol. 76:1212-1223; Sukovich et al. 2010. Appl. Environ. Microbiol. 76:3850-62; Friedman et al. 2008. International Patent WO2008/113041). Other Ole proteins were purified as described in the methods section and tested in admixture with OleA and with myristoyl-CoA as the substrate. Gas chromatography-mass spectrometry was used as it can detect both the OleA product following its decarboxylation to 14-heptacosanone and the expected olefin 14-heptacosene if the entire biosynthetic pathway were functional. FIG. III-5 shows that OleA and OleC in admixture produced only 14-heptacosanone (elution time 21.8), the product observed with OleA alone. However, when OleA was incubated with myristoyl-CoA, OleC and OleD, the olefin 14-heptacosene (20.4 min) was observed in addition to the peak corresponding to 14-heptacosanone. The identities of the products were confirmed by mass spectrometry.

To directly demonstrate that 2-myristoylmyristic acid was the intermediate giving rise to the olefin, we incubated synthetic 2-myristoylmyristic acid with OleC and OleD. The experiment yielded 14-heptacosanone and the expected olefinic product from head-to-head condensation, 14-heptacosene (FIG. III-6, peak A, 20.4 min). The identity of the compound was confirmed by the mass spectrum shown above the GC chromatogram in FIG. III-6A, with the major ion, m/z 378, representing the molecular ion.

Next, it was tested if the ketone 14-heptacosanone could also give rise to 14-heptacosene or any other discernible product. In this experiment (FIG. III-6B), OleC and OleD were incubated with 14-heptacosanone under the same conditions as described above and the olefinic product 14-heptacosene was not detected. A minor peak was observed at 20.35 min that had a mass spectrum different than that of 14-heptacosene (m/z 356). The small 20.35 min peak was also present in incomplete reaction mixtures that lacked OleD (FIG. III-6C), further demonstrating it is a contaminant and not relevant to olefin biosynthesis. It was found to be a minor impurity in the synthetic ketone preparation.

Overall, these data suggest that X. campestris OleA produced β-ketoacid intermediates from acyl-CoAs (C₈-C₁₈) and that the ketone is a non-physiological product arising from spontaneous decarboxylation. Note that ketones have been observed in vivo in recombinant bacteria containing heterologous OleA genes (Beller et al. 2010. Appl. Environ. Microbiol. 76:1212-1223; Sukovich et al. 2010. Appl. Environ. Microbiol. 76:3850-62). Additionally, Albro and Dittmer incubated ketones with crude protein fractions and failed to observe olefins (Albro et al. 1970. Biochemistry. 9:1893-1898). However, when a full suite of ole genes are present in native hosts, olefinic products, and not ketones, are typically observed (Sukovich et al. 2010. Appl. Environ. Microbiol. 76:3842-49). Thus, previous in vivo results are fully consistent with the in vitro data obtained in the present study.

### III-C. Discussion

OleA is homologous to proteins in the thiolase or condensing enzyme superfamily (Beller et al. 2010. Appl. Environ. Microbiol. 76:1212-1223; Sukovich et al. 2010. Appl. Environ. Microbiol. 76:3850-62). This is a very large superfamily of over 13,000 known proteins. The known thiolase superfamily proteins typically catalyze condensation reactions between acyl-thioester substrates, either with or without the loss of a carboxyl group. Approximately seventy bacteria are known to contain genes denoted as oleABCD and those tested produce long-chain olefinic hydrocarbons (Sukovich et al. 2010. Appl. Environ. Microbiol. 76:3850-62). The precise role of each ole gene product in the biosynthesis remains to be defined. When the oleC gene is deleted, or only the oleA gene is present in vivo, a long-chain ketone(s) is observed. These data supported the idea that OleA is involved in the initial stages of the head-to-head hydrocarbon biosynthetic reactions (Beller et al. 2010. Appl. Environ. Microbiol. 76:1212-1223; Sukovich et al. 2010. Appl. Environ. Microbiol. 76:3850-62; Friedman et al. 2008. International Patent WO2008/113041).

There are two alternative proposals in the literature regarding the OleA condensation reaction (FIG. III-1). Beller, et al (FIG. III-1A) proposed that OleA catalyzes a decarboxylative condensation between a β-ketoacyl-CoA and a fatty acyl-CoA (Beller et al. 2010. Appl. Environ. Microbiol. 76:1212-1223). Sukovich, et al (FIG. III-1B) have proposed that OleA catalyzes a non-decarboxylative Claisen condensation between two fatty acyl-CoA substrates (Sukovich et al. 2010. Appl. Environ. Microbiol. 76:3850-62). These two types of condensation reactions are difficult to differentiate in vivo where both fatty acyl-CoAs and β-ketoacyl-CoAs may be present simultaneously and many enzymes are present. The study by Beller and coworkers used a purified OleA enzyme, but their demonstration of activity required the addition of a crude soluble protein extract from Escherichia coli (Beller et al. 2010. Appl. Environ. Microbiol. 76:1212-1223). The proposed β-ketoacyl-CoA substrate was suggested to have been generated from the corresponding acyl-CoA by the proteins present in the E. coli soluble fraction. A clear differentiation between OleA reaction A and B could be obtained using a purified OleA preparation in admixture with defined substrates in vitro. The two types of condensation reactions could also be differentiated by determining the reaction product. OleA reaction A produces a 1,3-diketone while OleA reaction B yields a β-ketoacid.

There are other important questions that can be answered directly using a purified OleA protein and purified single substrates. These include determining the substrate specificity of OleA with respect to chain length, determining the complete reaction stoichiometry, determining what drives the apparent Claisen condensation to completion and revealing why cloning oleA genes in heterologous hosts produces monoketones. These issues are addressed herein.

The OleA protein from Xanthomonas campestris was cloned, overproduced in E. coli, and purified to homogeneity. The putative product of the reaction was synthesized chemically to allow comparison with the biochemical product. OleA was shown to react with myristoyl-CoA to produce the corresponding β-ketoacid via a non-decarboxylative Claisen condensation reaction. This intermediate was shown to react, in the presence of OleC and OleD, to yield a long chain olefin. In the absence of OleC and OleD, the product of the OleA reaction was shown to undergo spontaneous chemical decarboxylation to yield a ketone. This explains previous in vivo observations of ketone formation with the produceion of an oleA gene in a heterologous host (Beller et al. 2010. Appl. Environ. Microbiol. 76:1212-1223; Sukovich et al. 2010. Appl. Environ. Microbiol. 76:3850-62).

More specifically, in this study, the OleA protein from X. campestris was purified to homogeneity and shown to condense fatty acyl-CoA substrates to produce a condensed β-ketoacid with the release of two moles of CoA. The β-ketoacid, synthesized chemically or enzymatically, was shown to undergo further metabolism to yield a long-chain olefin in the presence of OleC and OleD. These studies confirmed that OleA catalyzes the first reaction in alkene biosynthesis with acyl-CoA substrates and carries out a non-decarboxylative Claisen condensation reaction.

An OleA protein was previously purified from Micrococcus luteus and it was proposed to catalyze a different reaction (Beller et al. 2010. Appl. Environ. Microbiol. 76:1212-1223) than the one demonstrated here with the OleA protein from Xanthomonas campestris. The Xanthomonas and Micrococcus OleA proteins showed 38% sequence identity (Table III-1) in a pairwise alignment of their amino acid sequences (Altschul et al. 1997. Nucleic Acids Res. 25:3389-3402) so they could conceivably catalyze different reactions. The oleA genes from both organisms cluster with oleBCD genes. In the Micrococcus genome, the oleB and oleC genes are fused and likely produce a multi-domain protein. However, the OleA, OleB, OleC and OleD domains are present in both organisms. It was shown in the present study that OleC and OleD proteins act on the β-ketoacid product generated by X. campestris OleA to produce a long chain olefin. When the Micrococcus oleA gene was cloned and produced in E. coli, long chain ketones were observed (Beller et al. 2010. Appl. Environ. Microbiol. 76:1212-1223). In the present study, the recombinant E. coli strain producing the X. campestris OleA protein alone was also observed to produce long-chain ketones that were not observed in the wild-type E. coli (data not shown). The in vitro data in this study showed that the ketones readily arise from the decarboxylation of a corresponding β-ketoacid intermediate. These observations are all consistent with a non-decarboxylative Claisen condensation as shown in FIG. III-1B and difficult to reconcile with the proposed decarboxylative reaction shown in FIG. III-1A.

The reaction catalyzed by OleA is somewhat reminiscent of the Zoogloea thiolase reaction that catalyzes the first step in the biosynthesis of polyhydroxybutyrate (Davis et al. 1987. J. Biol. Chem. 262:82-89). In the latter reaction however, the condensed product is β-ketoacetyl-CoA, acetoacetyl-CoA, and with OleA, the product is a β-keto acid. Several lines of evidence strongly suggested that OleA does not produce a β-ketoacetyl-CoA that is hydrolyzed to the acid by another enzyme. First, the oleA gene was cloned as a single open reading frame (ORF) from synthetic DNA and produced in E. coli, a bacterium that does not natively synthesize hydrocarbons. Enzymes capable of hydrolyzing 2-myristoylmyristoyl-CoA are not likely to be present in E. coli. Second, OleA was highly purified as shown by SDS-PAGE (FIG. III-2), so even the unlikely E. coli hydrolytic enzyme would have been removed. Lastly, our HPLC conditions would have detected 2-myrisotylmyristoyl-CoA and this was never detected.

Based on the data obtained, and the known role of the conserved cysteine found in other members of the thiolase superfamily, a working reaction mechanism can be presented for the OleA catalyzed reaction (FIG. III-7). We propose that initially an active site cysteine in the resting enzyme (FIG. III-7A) is acylated and coenzyme A is liberated (FIG. III-7B). Subsequently, the tethered substrate is likely activated by an active site base to yield a carbanion on the tethered substrate (FIG. III-7C). The carbanion then can react at the active site with the carbonyl carbon of a non-covalently bound acyl-CoA (FIG. III-7D). That reaction forms a carbon-to-carbon bond with the condensed product still tethered to the enzyme cysteine and producing the second molecule of coenzyme A formed in the reaction cycle (FIG. III-7E). The covalently-bound condensation product can then undergo hydrolysis to yield the final β-ketoacid product and regenerate the free cysteine residue of the resting enzyme state (FIG. III-7A). While several features of this proposed mechanism are not yet demonstrated directly, there are multiple data that support this proposal. First, this mechanism explains the observed stoichiometry in which two moles of coenzyme A are observed per mole of condensed product. Secondly, the observed high rate of hydrolysis of acyl-CoAs to produce fatty acids is not unexpected if the enzyme has a mechanism to hydrolyze thioester-linked intermediates during its normal reaction cycle. Thus, there could be a kinetic competition between hydrolysis of the initially-bound acyl-group (FIG. III-7B) and the tethered condensation product (FIG. III-7E). Depending upon the binding affinity for the different length acyl-CoA used in the experiment described in Table III-3, hydrolysis of intermediate 7B or 7E would occur preferentially. Lastly, proteins in the thiolase superfamily typically use an active site cyteine to acquire an acyl chain to initiate catalysis (Heath et al. 2002. Nat. Prod. Rep. 19:581-596; Haapalainen et al. 2006, TRENDS Biochem. Sci. 31:64-71) and the region around the cysteine residue shown in Table III-1 is the most highly conserved region of OleA with other members of the superfamily.

There are significant questions that remain to be addressed regarding this proposed mechanism (FIG. III-7). First, the identity of the proposed cysteine nucleophile has not been directly demonstrated here. Second, the suggested generation of a carbanion (FIG. III-7B) requires a general base that remains to be identified. Additionally, this mechanism would be supported by the identification of the binding sites for the acyl chains and showing that the chains are covalently and non-covalently bound, respectively.

This study identified the product of the OleA-catalyzed reaction to be β-keto acid. The production of olefins required the presence of OleC and OleD, in addition to OleA. These data indicated that OleC and OleD catalyze further reactions with the β-ketoacid intermediate generated by OleA. This was supported by experiments in which 2-myristoyl myristic acid was transformed to an olefin by OleC and OleD. The corresponding ketone was not transformed to an olefin, consistent with the idea that the ketone is not a physiologically-relevant intermediate. There is also the issue that C-2 in 2-myristoyl myristic acid is a chiral center. The synthetic 2-myristoyl myristic acid is racemic and it is plausible that only one enantiomer will react with OleD. The chirality of the reaction is currently under investigation.

### IV. Strain Improvement

Referring to FIG. IV-1, synthetic oleA gene encoding the amino acid sequence of OleA from Xanthomonas campestris spv. campestris str. ATCC 33913 (NP_{—}635607.1) was cut with NdeI and BamHI restriction enzymes and cloned into pET28b+ (Novagen, Madison, Wis.). The gene was transformed into E. coli One Shot BL21 (DE3) (Invitrogen). Soluble protein production was screened in 50 ml cultures induced for 4 hr at 37° C. OleA produced well and the X. campestris OleA obtained from E. coli was found to be active in vitro, and that was selected for further study.

### V. Chloroflexus Cloning and Produceion

### V-A. Materials and Methods

Chloroflexus aurantiacus was routinely grown in Chloroflexus media at 55° C. (3). Primers used in this study are listed in Table 5.1 (Table V-1). The Chloroflexus aurantiacus oleA was amplified using primers ClthiolCompF and ClthiolCompR containing the SpeI and SacI restriction sites. Resulting PCR products were ligated into the Strataclone cloning system (Agilent Technologies) followed by ligation of the product into the pBBR1MCS2 produceion vector. The oleA was also amplified using primers RethiolF and RethiolR containing the ClaI and XhoI restriction sites. Resulting PCR products were ligated into the Strataclone system followed by ligation of the product into the pBBR vector provided by the Srienc Laboratory (University of Minnesota). Vector constructs were introduced into E. coli WM3064 and conjugated into the ole deletion or wild-type S. oneidensis MR-1 strain (pBBR1MCS2 vector) or the PHB-deficient strain of R. eutropha (pBBR vector). Appropriately oriented inserts were verified by PCR analysis. For hydrocarbon analysis, cultures were extracted using the Bligh and Dyer technique (Frias et al. 2009. Appl. Environ. Microbiol. 75: 1774-1777) prior to GC-MS analysis. Hexadecane spikes were routinely added to extracts for hydrocarbon quantification. Bacterial constructs were routinely grown at 30° C. unless stated otherwise.

TABLE V-1
Strains, vectors, and primers used in this study

| Strains: | Notes | Reference |
|---|---|---|
| Chloroflexus aurantiacus | wildtype | |
| sp. J-10-fl | | |
| Shewanella oneidensis | oleABCD knockout | |
| Δole | | |
| Ralstonia eutropha | strain unable to produce PHB | |
| Plasmids: | | |
| pBBR1MCS2 | 5.1 KB broad-host range plasmid, | |
| | lacZ.Km^{r} | |
| pChloro | pBBR1MCS2 containing 1.1 KB fragment | Chapter 3 |
| | of oleA | |
| pBBR | vector obtained from Scrienc Labo- | |
| | ratory (University of Minnesota) | |
| pBBT Chloro | pBBR containing 1.1 KB fragment | This study |
| | of C. aurantiacus oleA | |
| Primers | | |
| ChlorooleAClaF | CATATTATCGATATGCTATTCAGGCATGTCATGATCG | |
| ChlorooleAXhoR | CAATATCTCGAGTCACCACGTCACACTCATCATTGAAC | |

Chapter 3 refers to that chapter in D. Sukovich. 2010. Ph. D. dissertation. University of Minnesota, Twin Cities.

### V-B. Results and Discussion

When Chlorofluxus aurantiacus was grown under optimal conditions for three weeks, it was found that the organism produced one hydrocarbon (FIG. V-1). This hydrocarbon contained a parent ion of m/z 430, consistent with the hydrocarbon 9,15,25-hentriacontatriene.

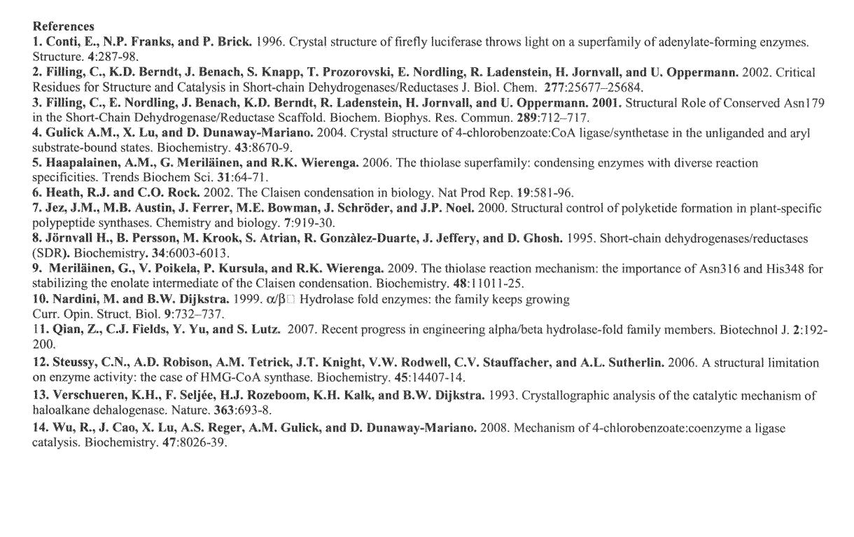

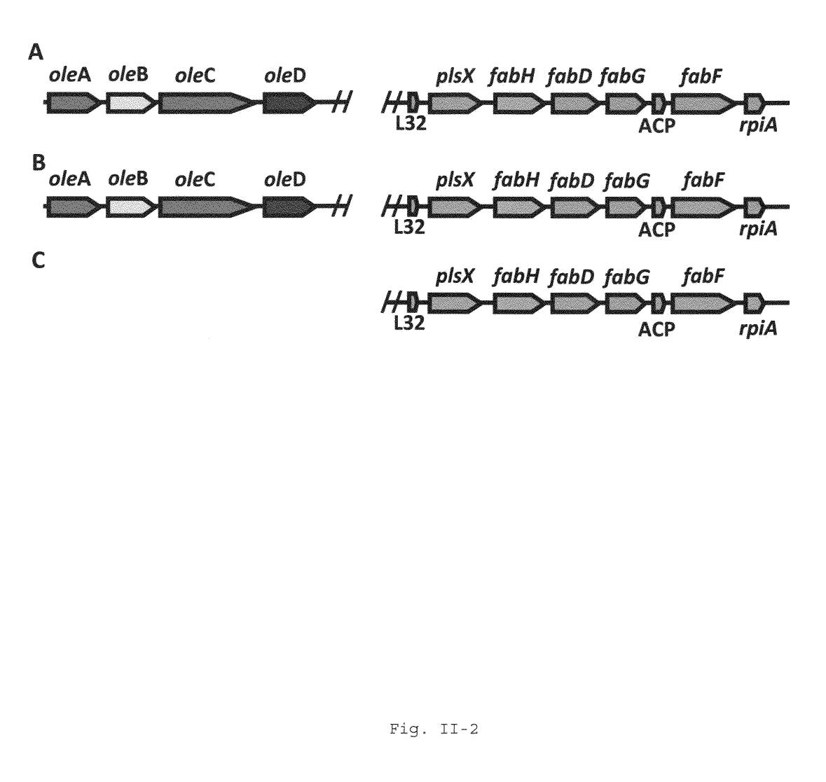

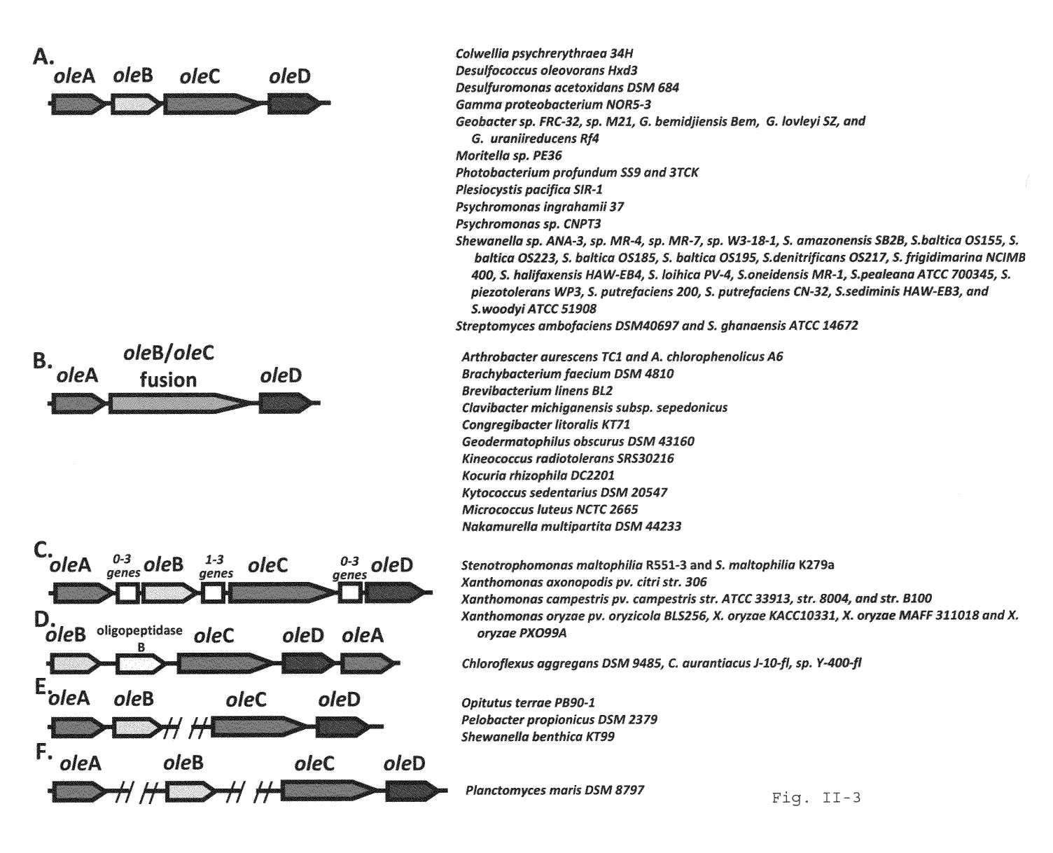

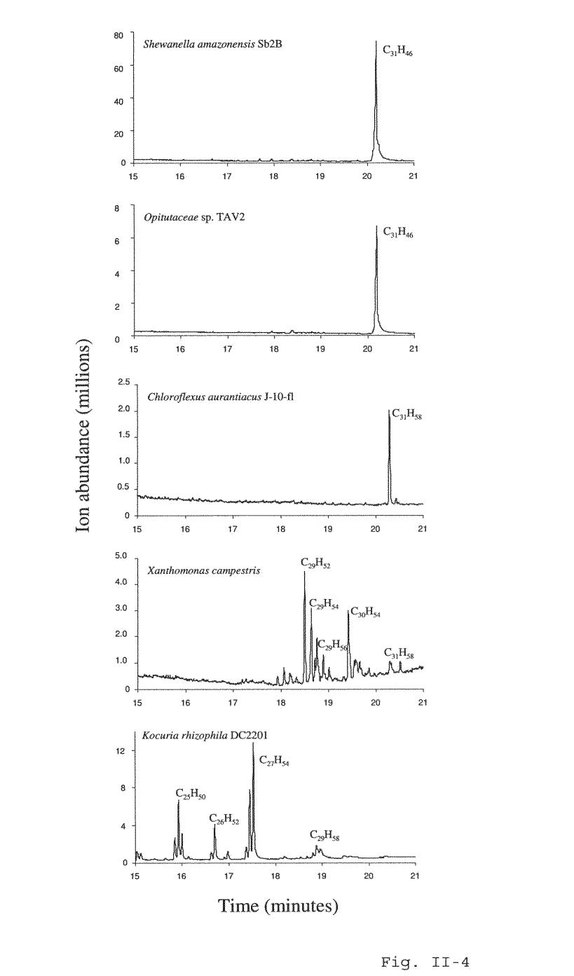

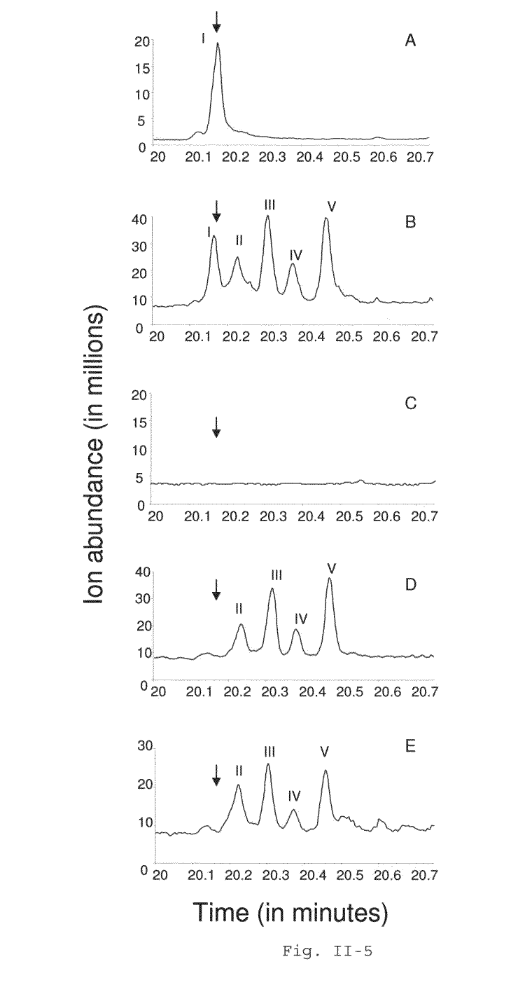

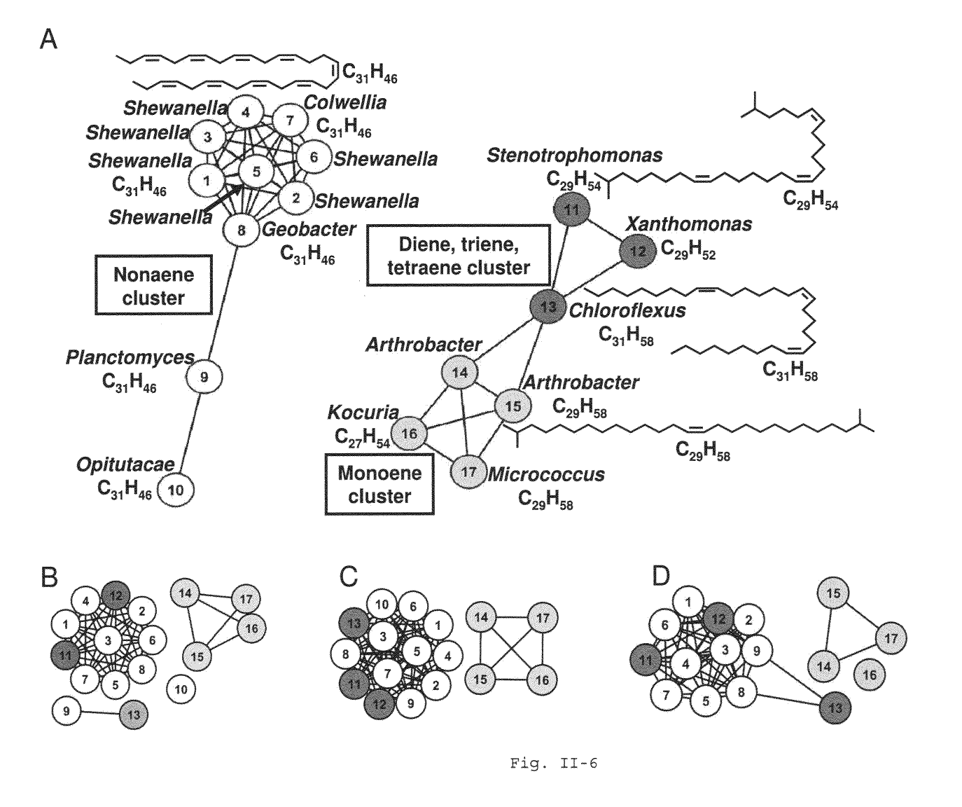

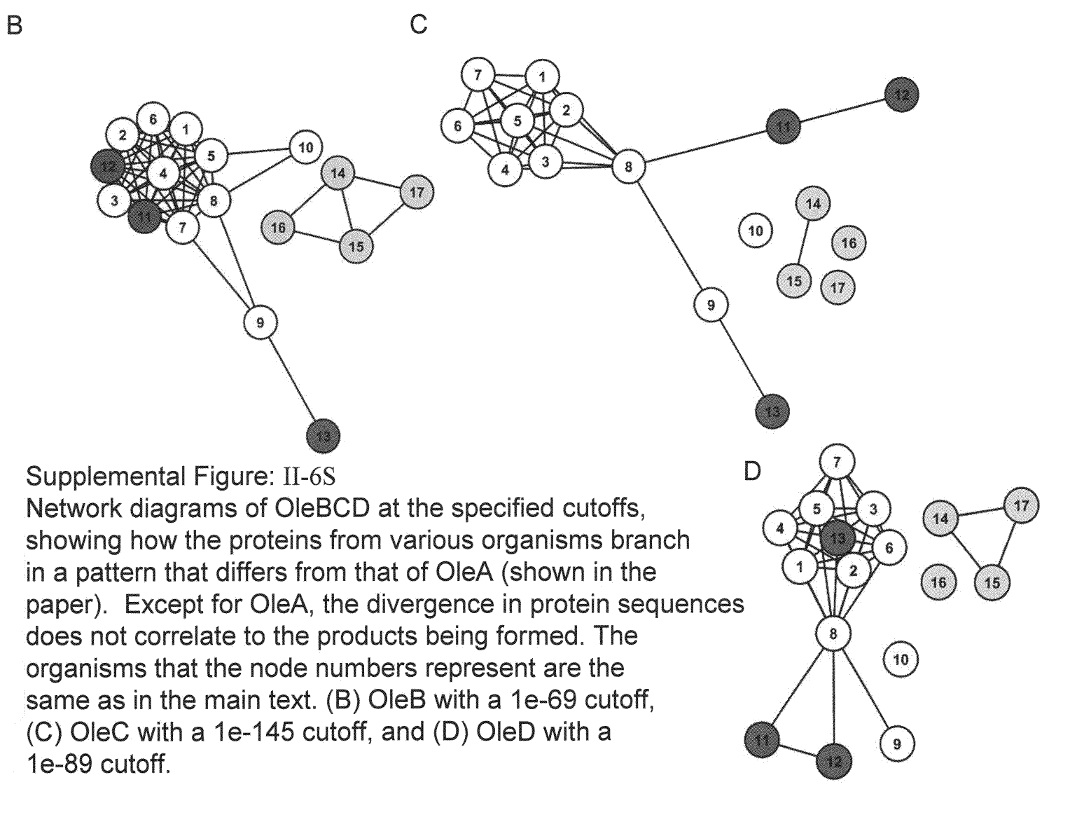

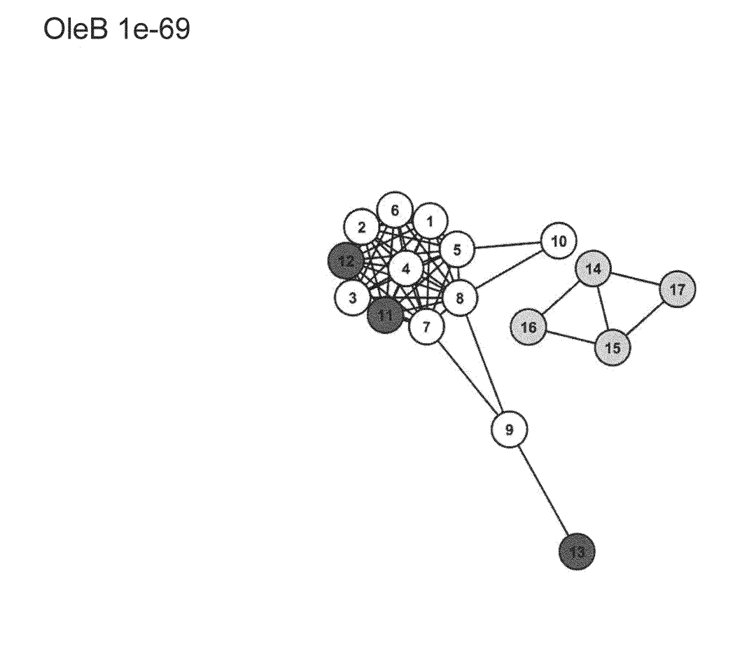

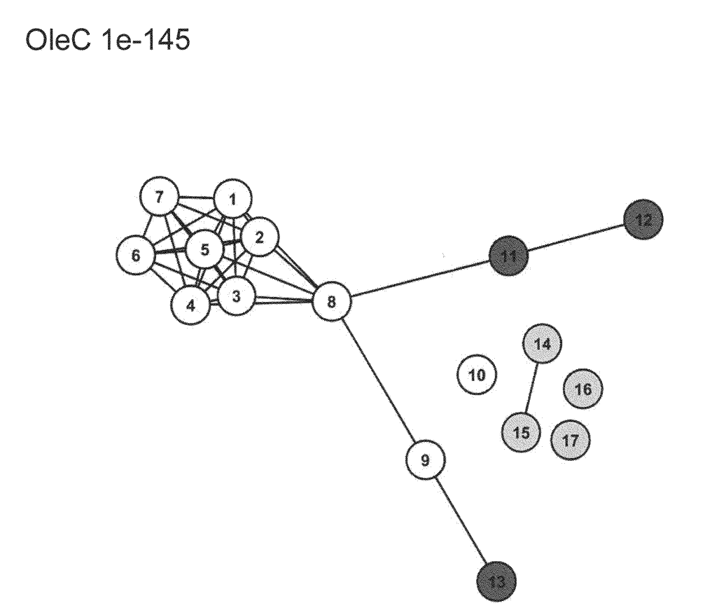

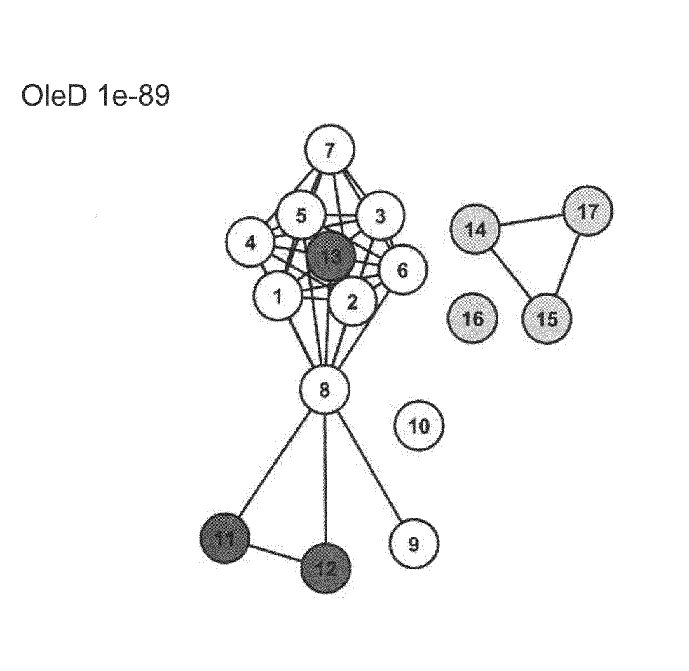

When the C. aurantiacus oleA was introduced into S. oneidensis Δole, it was found that the organism not only produced the expected C31-ketodiene, but also numerous other ketones ranging from 29 to 31 carbons in length (FIG. 5.2). The predominant ketone produced contained a fragment with m/z 223 and a parent ion of m/z 418. This mass spectrum is consistent with a compound containing a carbonyl functionality directly in the middle of a C29 chain flanked by two C14 chains, each containing one double bond. Another predominant compound showed fragments of m/z 223 and 225 and a parent ion of m/z 420, consistent with a compound containing a carbonyl functionality directly in the middle of a C29 chain with 14 saturated carbon atoms on one side and a C14 chain with one double bond on the other. Positional isomers were also noted in the gas chromatogram. A similar spectra of compounds were seen for ketones of 30 and 31 carbons in length, but in lesser quantities.

Various oleAs were introduced into Ralstonia eutropha (Table V.1 S) and though all oleAs were transcribed as found by RT-PCR only the C. aurantiacus oleA was found to produce identifiable products (FIG. V-3). Gas chromatography-mass spectroscopy analysis revealed that the R. eutropha strain containing the C. aurantiacus produced predominantly a product with a parent ion of m/z 390 with a strong ion peak at m/z 209. This mass spectrum is consistent with a compound containing a carbonyl functionality directly in the middle of a C27 chain flanked by two C13 chains, each containing one double bond. It also had a strong peak with a parent ion of m/z 392 with secondary ions of m/z 209 and 211, consistent with a compound containing a carbonyl functionality directly in the middle of a C27 chain with 13 saturated carbon atoms on one side and a C13 chain with one double bond on the other. A third peak identified as a saturated ketone of 27 carbons was also detected (m/z 394, 211). Other minor ketones were identified, all of which were 29 carbons in length. These included two isoforms of a ketone with two unsaturation locations (m/z 418, 223).

Previous studies showed that OleA condensed two fatty acyl-CoAs to produce a fatty-acyl compound. When OleBCD is not present, the fatty-acyl compound is spontaneously decarboxylated to produce a ketone (Frias et al. 2011. J. Biol. Chem. 286(13):10930-8). Whereas all the C31:9-producing OleAs group together when analyzed as a network diagram, the C. aurantiacus OleA groups with the Xanthomonas campestris and Stenotrophomonas maltophilia OleAs. When produced in the S. oneidensis Δole background, the S. maltophilia OleA condenses numerous fatty acids to produce ketones. Similarly, X. campestris and S. maltophilia both produce numerous alkenes in various chain-lengths naturally.

Chloroflexus aurantiacus produces predominantly palmitoyl-CoA, and only minor amounts of the other long-chain fatty acids (van der Meer et al. 2001. J. Biol. Chem. June 15; 276(24):10971-6). Therefore, in the protein's natural environment, the OleA may be saturated by the C16-fatty acid and any alternative condensation occurrences may not be noticed in the GC-traces. In contrast, S. oneidensis produces predominantly C15 fatty acids (Abboud et al. 2005. Appl. Environ. Microbiol. 71(2):811-6) while R. eutropha had been previously shown to produce predominantly C₁₋₄ saturated and monounsaturated fatty acids. If these fatty acids were condensed, the resulting ketone would be 29 or 27 carbons in length respectively and contain parent ions of m/z 420-424 and 392-396.

TABLE V-1S
Strains, vectors and primers refered to above

| Strains: | Notes | Reference |
|---|---|---|
| Chloroflexus aurantiacus | wildtype | 120 |
| sp. J-10-fl | | |
| Shewanella oneidensis | oleABCD knockout | 150 |
| Δole | | |
| Ralstonia eutropha | strain unable to produce PHB | 72 |
| Plasmids | | |
| pBBR1MCS2 | 5.1 KB broad-host range plasmid, lacZ. | 89 |
| | Km^{r} | |
| pChloro | pBBR1MCS2 containing 1.1 KB fragment | Chapter 3 |
| | of oleA | |
| pBBR | vector obtained from Scriene Labo- | |
| | ratory (University of Minnesota) | |
| pBBR Chloro | pBBR containing 1.1 KB fragment | This study |
| | of C. aurantiacus oleA | |
| pBBRSm | pBBR containing 1.1 KB fragment | This study |
| | of S. maltophilia oleA | |
| pBBRXanth | pBBR containing 1.1 KB fragment | This study |
| | of X. campestris wild type oleA | |
| pBBRCong or Syn | pBBR containing 1.1 KB fragment | This study |
| | of Synthesized C. litoralis oleA | |
| pBBRXanSyn | pBBR containing 1.1 KB fragment | This study |
| | of synthesized X. campestris oleA | |
| pBBRXylSyn | pBBR containing 1.1 KB fragment | This study |
| | of synthesized Xylella fastidiosa oleA | |
| pBBRPpacificaSyn | pBBR containing 1.1 KB fragment | This study |
| | of synthesized P. pacifica oleA | |
| pBBRgpSyn | pBBR containing 1.1 KB fragment | This study |
| | of gamma proteobacteria Nor-5 oleA | |
| Primers | | |
| S.m. CompFClaI | ATCTATCGATAACCTCGATGCTCTTCAAGAATGTCTC | |
| S.m. CompRXhoI | CGATCTCGAGGAAGATCATCGCTGTCCGTCGCGAGC | |
| ChlorooleAClaF | CATATTATCGATATGCTATTCAGGCATGTCATGATCG | |
| ChlorooleAXhoR | CAATATCTCGAGTCACCACGTCACACTCATCATTGAAC | |
| XantholeANarIF | ATTAATGGCGCCATGCTCTTCCAGAATGTCTCCATCGC | |
| XantholeAApaIR | AATATTGGGCCCTCACCAAACCACTTCGGCCATCGA | |
| CongoleANarIF | AATATTGGCGCCATGTCTGGTAACGCTAAATTCACT | |
| CongoleAXhoIR | TTAATACTCGAGCTACCAAGCGATTTCTAAAGCCAT | |
| XanSynoleANarIF | AATATTGGCGCCATGTTATTCCAAAACGTTTCTATC | |
| Xan/XylSynoleAXhoIR | TTAATACTCGAGCTACCAAACAACTTCAGCCATAGAAC | |
| XyloleANarIF | AATATTGGCGCCATGTTATTCAACAACGTTTCTATC | |
| PlesioleANarIF | AATATTGGCGCCATGCGTTTCGCTAACGTTTCTATC | |
| PlesioleAXhoIR | TTAATACTCGAGCTACCAAACAACTTCAGCCATAGCAC | |
| gammaoleANarIF | AATATTGGCGCCATGCACTTCGAATCTGTTGTTATC | |
| gammaoleAXhoIR | TTAATACTCGAGCTACCAAACAACTTCAGCCATAGCA | |

Refs in Table V-1S: Chapter 3 refers to that chapter in D. Sukovich. 2010. Ph.D. dissertation. Uof MN; 72. Jackson et al. 1998. Thesis UofMN; 89. Kovach et al. 1995. Gene 166: 175-176; 120. Pierson et al. 1974. Arch. Microbiol. 100: 5-24; 150. Sukovich et al. 2010. Applied and Environmental Microbiology 76: 3842-3849.

### VI. A Cyanobacterium Supports the Growth of a Heterotroph

To maintain the co-culture of the cyanobacterium (Synechococcus) and the heterotroph (Shewanella), a minimal medium was established that would support growth of both Shewanella and Synechococcus. We started with the preferred medium for Synechococcus, known as BG11. This medium did not prove to be sufficient for growth of Shewanella. A modified Synechococcus minimal medium, denoted here as BG11AN medium, was used and supplemented with lactic acid to test both lactate inhibition and ammonia inhibition. BG11AN has ammonium nitrate at 1.5 g/L replacing the sodium nitrate used in BG11. Shewanella grew well in BG11AN medium. The Synechococcus grew in the BG11AN medium, not as well as in BG11, but acceptably. The cultures in BG11 AN were more of a yellow green than the deep green of the BG11 culture. Lactic acid did not inhibit growth at levels of 1 g/L or less. In fact, it stimulated growth at levels up to 0.5 g/L. It was concluded that the BG11AN medium can be used for co-culture of Synechococcus and Shewanella in subsequent experiments.

The effects of growth supplements of the type found in corn steep liquor on the growth of Synechococcus and Shewanella were examined. It was found that these supplements, consisting largely of amino acids, singly or in combination, did not inhibit Synechococcus and promoted the growth of Shewanella. In fact, flasks supplemented with glutamate, glutamine and casamino acids gave slightly better growth of Synechococcus.

It was necessary to have Synechococcus strains that fix CO₂ and transform that carbon into secreted products. Three such strains of Synechococcus elongates were examined: (1) an ldhA lldP UdhA strain that produces lactate dehydrogenase, the lactate transporter to excrete lactate, and a transhydrogenase to supply NADH to sustain pyruvate reduction to lactate, (2) an ldhA lldP strain that produces lactate dehydrogenase and the lactate transporter, and (3) a GLF InvA strain that produces the glucose/fructose transporter and invertase, and secretes fructose and glucose. These strains were kindly provided by Drs. Pam Silver and Jeffrey Way at Harvard Medical School. The first strain was studied extensively but found to be too unstable. The source of the instability was the presence of the heterologously produced UdhA (NADPH-NADH transhydrogenase) that depleted NADPH pools and thus greatly diminished key biosynthetic pathways. This phenotype imparts a very strong selective pressure against the presence or produceion of the NADPH-NADH transhydrogenase gene. Subsequently, studies have focused on the IdhA lldP strain that secretes lactate and the GLF InvA strain that secretes fructose and glucose.

Concomitant with this, a fourth requirement was to obtain complementary Shewanella strains. The initial Shewanella strain grew well with lactate as the carbon source but not with glucose or fructose. A recent paper described the deficiency in the ability to utilize sugars (Pinchuk et al. 2010. PLoS Comput. Biol. 6(6):e1000822). The wild-type organism has the metabolic pathways to use sugars but it lacks an operational transporter. Genome sequencing had revealed that the transport gene is present but had an apparent frame-shift to render the transporter non-functional. To overcome this problem, Shewanella mutants that had the ability to grow on glucose or fructose were selected. Subsequently, Shewanella strains were obtained that would grow on either sugar as a carbon source. Thus, we now had a suitable Shewanella strain that would take up glucose and fructose, catabolize those sugars, and use the carbon to produce ketones from the heterologously produced OleA protein.

In the key experiment showing carbon transfer, the Synechococcus GLF InvA strain was grown in the minimal medium shown previously to support both Synechococcus and Shewanella strains. In addition, the medium was supplemented with 200 mM NaCl which had been previously shown to lead to enhanced sugar production and excretion. The medium was screened and comparable levels of sugars were excreted as described previously. The Synechococcus cells were then removed by centrifugation. The culture supernatant was then used as a growth medium for the Shewanella mutants shown previously to grow on glucose and fructose. Growth of this strain was observed, demonstrating carbon transfer to the Shewanella.

In additional experiments, we sought to show carbon transfer in cultures containing both Synechococcus and Shewanella simultaneously. This requires an ability to monitor each bacterial strain in the presence of the other. To investigate that, cultures of Synechococcus and Shewanella were mixed that been grown separately and examined the mixed culture under a fluorescence microscope. The method of visualization used: (1) 4′,6-diamidino-2-phenylindole (DAPI)

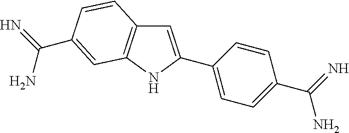

that stains all bacteria; and (2) the autofluorescence of the Synechococcus cells. In this manner, selectively Shewanella (DAPI stained) and Synechococcus (autofluorescence) were observed. Moreover, there was a significant difference in size of the two cell types, as shown in FIG. VI-1.

### VII. Example Nucleic Acid/Vector Constructs

Gene deletions were made using homologous recombination between flanking regions of oleA cloned into a suicide vector, pSMV3 (Saltikov et al. 2003. Proceedings of the National Academy of Sciences of the United States of America 100:10983-10988). Briefly, by using oleASoF1, oleASoR1, oleASoF2, and oleASoR1, the upstream and downstream regions surrounding the gene were cloned using the restriction sites SpeI and BamHI into the suicide vector in a compatible E. coli cloning strain (UQ950) (133). This plasmid was transformed into an E. coli mating strain (WM3064) (Saltikov et al. 2003. Proceedings of the National Academy of Sciences of the United States of America 100:10983-10988) and then conjugated into MR-1. While E. coli was commonly grown at 37° C., when S. oneidensis was present cells were incubated at 30° C. The initial recombination event was selected for by resistance to kanamycin. Cells containing the integrated suicide vector grew in the absence of selection overnight at 30° C. and then were plated onto LB plates containing 5% sucrose (Saltikov et al. 2003. Proceedings of the National Academy of Sciences of the United States of America 100:10983-10988). Cells retaining the suicide vector were unable to grow due to the activity of SacB, encoded on the vector, while cells that underwent a second recombination event formed colonies. Colonies were then screened by PCR to determine strains containing the deletion. The oleABCD gene cluster deletion of S. oneidensis MR-1 was created as described previously (Sukovich et al. 2010. Applied and Environmental Microbiology 76:3842-3849).

Complementation of the S. oneidensis oleA mutant was performed using the pBBR1MCS-2 produceion vector (Kovach et al. 1995. Gene 166:175-176) and the endogenous lac promoter (which is constitutive in MR-1 due to the absence of lad). Primers oleASoFcomp and oleASoRcomp containing SacI and SpeI restriction sites were designed for the regions flanking the ends of oleA. Resulting PCR products were ligated into the Strataclone cloning system (Agilent Technologies), followed by digestion and ligation of the product into the pBBR1MCS-2 produceion vector. The Stenotrophomonas maltophilia oleA gene was introduced into pBBR1MCS-2 as described previously (Sukovich et al. 2010. Applied and Environmental Microbiology 76:3842-3849). Constructs were introduced into E. coli WM3064 prior to conjugation with the oleA deletion, the ole cluster deletion, or wild-type MR-1 strains. All constructs were verified through PCR and sequencing analysis. Following conjugation, all constructs were maintained using 50 μg/ml kanamycin.

VIII. Increasing Ketone and Hydrocarbon Production in Shewanella oneidensis

Overproduceion of key genes in fatty acid synthesis. The enzyme responsible for the condensation reaction that yields hydrocarbons in Shewanella oneidensis (MR-1) is OleA. Homology searches have identified an OleA homologue in Stenotrophomonas maltophilia capable of condensing varying chain lengths of acyl-CoAs derived from fatty acid synthesis. The ability of this homologue to use multiple substrates from fatty acid synthesis provides an opportunity to increase hydrocarbon production. MR-1 and E. coli share homologues for the fatty acid synthesis pathway allowing metabolic engineering strategies in E. coli to be applied to metabolic engineering in MR-1. Key genes in fatty acid synthesis include acetyl-CoA carboxylase which catalyzes the first committed step in fatty acid synthesis which converts acetyl-CoA to malonyl-CoA. Increasing produceion of this enzyme has been shown to increase fatty acid production. A modified periplasmic thioesterase named TesA that has the N-terminal leader sequence removed causes localization to the cytoplasm effectively removing ACP from long chain acyl-ACPs yielding free fatty acids and eliminating feedback inhibition in E. coli. The resulting long chain free fatty acids can then be activated by acyl-CoA ligases (FadD) yielding long chain acyl-CoAs that are the substrate for oleA. These genes have been cloned into plasmids and produced. It is possible that TesA will be an essential enzyme to remove feedback inhibition from fatty acid synthesis and also link fatty acid synthesis to ketone and hydrocarbon synthesis since most flux from fatty acid synthesis is targeted for lipid synthesis.

Genes of interest that have been produced. accABCD: acetyl-CoA carboxylase tesA: thioesterase A; fadD-1: acyl-CoA ligase.

Plasmids used. There are several options for plasmids that can be used in produceion of the genes mentioned in the above paragraph. A list of these plasmids is below with key features of each mentioned. Plasmids pBBR and pBBAD producing oleA have been used Hydrocarbon extraction data indicates that induction of oleA from the arabinose promoter produces more hydrocarbons than oleA produced from the constitutive lac promoter in pBBR. The above-mentioned genes have also been cloned into pUCBB and pBBR-BB plasmids. Each gene has also been cloned and sequence verified in Biobrick plasmids, each under control of the lac promoter. Different combinations of the genes are now being combined to test for hydrocarbon production. Preliminary results from oleA produced in the high copy number pUCBB suggest that the protein has toxic effects at high produceion levels.

An additional method used to increase hydrocarbon synthesis involved cloning a highly active mutant lac promoter into a Biobrick plasmid.

pBBR: low-medium (˜100/cell) copy number, constitutive lac promoter
pBBAD: low-medium copy number, arabinose inducible promoter
pUC-BB: high copy number (˜1000/cell), constitutive lac promoter, Biobrick
pBBR-BB: low-medium copy number, constitutive lac promoter, Biobrick

Explanation of Biobrick plasmids. Traditional cloning is used to insert a gene into the multiple cloning site downstream of the lac promoter. Restriction enzyme cut sites that are complimentary but not palindromic are placed upstream of the lac promoter and downstream of the terminator (xbaI and speI for example). Restriction digestion with these enzymes removes the gene of interest along with lac promoter and terminator. This “biobrick” can then be cloned into another biobrick plasmid that has been cut once with speI. Complimentary ends to the 5 prime sticky end match xbaI on the biobrick and complimentary ends to the 3 prime sticky end match speI on the biobrick allowing ligation into the plasmid and removing the restriction site between the two genes. Cutting with speI can be repeated to add additional biobricks so that one plasmid contains many genes each under control of its own promoter. This technique is being utilized to overproduce genes designed to increase acyl-CoA pools.

Deletion of genes in Shewanella to boost ketone/hydrocarbon production. Deletion methods were used to remove the native olefin pathway (Δole) responsible for hydrocarbon synthesis and also genes for making polyunsaturated fatty acids (Δpfa). The latter pathway consumes acetyl-CoA and thus robs carbon from ketone/hydrocarbon biosynthesis. All strains are tested for hydrocarbon synthesis with plasmids producing oleA from Stenotrohomonas maltophilia. The first deletion made in MR-1 was the acyl-CoA dehydrogenase (fadE). FadE catalyzes the first committed step in fatty acid degradation and is an excellent target for blocking the degradation of the pools of long chain acyl-CoAs that we want condensed by OleA. Deletion of acyl-CoA ligase (fadD) was made to determine the effect on hydrocarbon synthesis since OleA requires a CoA activated fatty acid. Hydrocarbon levels appear lower in fadD mutant strains than those of wild type or in the fadE knockout. We expect the ΔfadE and ΔfadE phenotypes to be more drastic once the fatty acid overproduceion genes are being produced in MR-1. The amount of long chain free fatty acids or CoA activated long chain fatty acids would be much lower in strains not overproducing genes designed to increase fatty acid synthesis.

Computational modeling of metabolism to boost ketone/hydrocarbon production in Shewanella. This was done to improve hydrocarbon yield, and as a result final titer, by utilizing rational strain design to direct the deletion or overproduceion of genes involved in central metabolism. Based on our computational modeling, called elementary mode analysis, we identified several gene deletions that can improve ketone/hydrocarbon production in Shewanella. See Table VIII-1 and FIG. VIII-1. Of these, we decided to focus on lactate consumption specifically, because this is both the preferred substrate of S. oneidensis, and because we currently possess a Synechococcus strain that secretes lactate. Of the genes identified in Shewanella, we specifically focused on ΔsfcA, ΔpckA, ΔgcvT, Δpta, ΔpykA for several reasons. Δzwf1 was not pursued because fluxomics performed by Tang et al. in 2007 in J. Bacteriol. 189(3):894-901 showed that pentose phosphate pathway under lactate consumption represents a small fraction of total flux, meaning that any interruption would have minimal effect on total product yield. In addition, previous lab members had experienced difficulty in deleting the gene ndh, so the pre-existing deletions (ΔpckA, ΔgcvT, Δpta, ΔpykA) were focused on for this research. Briefly, we expect ΔpckA, and ΔpykA to improve product yield by reducing carbon and energy loss to futile/indirect synthesis pathways, in this case anaplerotic reactions. In addition, ΔgcvT, Δpta and related gene deletions ΔackA and Δacs are expected to improve yield by preventing acetate secretion, and glycine degradation to formate (ΔgcvT only).

TABLE VIII-1
Gene knockouts identified in different carbon source conditions

| lactate | Acetate | NAG |
|---|---|---|
| Δzwf1 | Δzwf1 | Δgnd1 |
| Δndh | Δndh | Δndh |
| ΔsfcA | ΔsfcA | ΔsfcA |
| ΔpckA | ΔpckA | ΔpckA |
| ΔgcvT | ΔgcvT | ΔgcvT |
| Δpta | Δpta | Δpta |
| ΔpykA | ΔpykA | ΔpykA |
| | | Δfbp |

The first attempt to analyze HC production in single knockout strains grown on minimal medium was not successful. This was because the cell density in SBM peaks at 0.5-0.6 OD, reducing HC resolution below detection limits (note: detection levels seem to be improved with the new GC/MS). Minimal medium is where we expect our mutations to exert the most force in terms of influencing metabolic flux.

As a result, 5 ml cultures of each strain were cultivated in LB (in triplicate to quintuplet), with 3 ml of this medium being extracted at 48 hrs. incubation and extracted using the method described by Frias et al. 2009. Appl. Environ. Microbiol. 75(6):1774-7. The present protocol differed from his only in that we prepared a 1/100 dilution of hexadecane standard in heptane. Then, instead of pipetting 0.5 ul of pure standard, we pipetted 50 ul, apparently reducing error between samples.

Also at first, we hoped to make single knock strains, assess HC improvements at each additional deletion, and iteratively generate improved strains. However, the production differences between single knockout strains and wild type (and each other) proved to be smaller than the variation between samples, limiting our ability to design strains in this way. We then relied on the predictions of EM analysis, and generated combined deletion strains ΔpykA, Δack, Δpta, and either ΔpckA, or ΔgcvT. These strains were compared with wild type and another multiple KO strain, Δack, Δpta, Δald, Δacs, ΔgcvT and cultivated on LB as described above. All strains were transformed for HC production with plasmids pBBR1-MCS2-oleA or pBBR1-BB-oleA. The result of this production study, after 48 hrs of incubation at 30° C. and 200 rpm in 5 ml LB, are shown in FIG. VIII-2.

The graph in FIG. VIII-2 shows that the strains lacking acetate secretion, and with the ΔpykA deletion, which eliminates PEP kinase catalyzed conversion of PEP to pyruvate, leads to a ˜6-7 fold improvement in HC titer relative to both wild-type and the strain lacking gcvT and the potential acetate producing genes ald, ack, pta, and acs. The large difference in production titer observed here suggests that major metabolic gains are the result of a reduction in futile cycles and elimination of acetate secretion, rather than acetate secretion alone. It also appears that gcvT deletion has little to no effect on final yield. This is not entirely unexpected, as the carbon flux through gcvT under lactate utilizing conditions was observed to range between 1.1% and 5.5% of total carbon (Tang et al. 2007. J. Bacteriol. 189(3):894-901).

IX. Production of OleA in E. coli

Synthetic oleA genes were designed based on oleA genes from Congregibacter litoralis KT71 (ZP_{—}01103251.1), Xanthomonas campestris spv. campestris str. ATCC 33913 (NP_{—}635607.1), Xylella fastidiosa 9a5c (NP_{—}299252.1), Plesiocystis pacifica SIR-1(ZP_{—}01906524.1), and γ-proteobacterium NOR5-3 (ZP_{—}05127044.1), see supplementary figure S1, and purchased from DNA 2.0 (Menlo Park, Calif. The genes were cut with NdeI and BamHI restriction enzymes and cloned into pET28b+ (Novagen, Madison, Wis.). All 5 genes were separately transformed into E. coli One Shot BL21 (DE3) (Invitrogen). All five recombinant strains were screened for soluble protein produceion in 50 ml cultures induced for 4 h at 37° C. Two of the five constructs produced soluble protein in E. coli, only X. campestris was found to be active in vitro, and that was selected for further study.

X. campestris for OleA purification was cultivated under two different conditions. Small-scale cultivations were conducted in 2 L flasks containing 500 ml LB with 50 μg/ml kanamycin and induced at an OD₆₀₀ of 0.7-0.85 with 0.1 M isopropyl-β-D-thiogalactopyranoside (IPTG). After 4 h, cells were harvested by centrifugation for 25 min at 3000 g. Large-scale cell cultivation was conducted in the Biotechnology Resource Center, University of Minnesota. A 440 L culture was prepared in a 550 L DCI bioreactor (DCI-Biolafitte, St. Cloud, Minn.) using a Rhapsody digital controller system and induced with 0.5 mM IPTG.

Cells were extracted with 250 μl ethyl acetate using 16-hentriacontanone ketone (Tokyo Kasei Kogyo Co., Ltd., Japan) as an internal standard. After vortexing and 5 min of gentle centrifugation, the top solvent layer was transferred to a glass vial and analyzed using a gas chromatograph equipped with a flame ionization detector HP 7890A (Hewlett Packard, Palo Alto) and mass spectrometer HP 5975C (GC-MS-FID). GC was conducted under the following conditions: helium gas, 1.75 ml/min; HP-1 ms column (100% dimethylsiloxane capillary; 30 m by 250 μm by 0.25 μm); temperature ramp, 100 to 320° C.; 10° C./min, hold at 320° C. for 5 min, 250° C. injection port, and split at the outlet between MS and FID. The mass spectrometer was run under the following conditions: electron impact at 70 eV and 35 μA. The flame ionization detector was set at 250° C. with hydrogen flow set at 30 ml/min, air set at 400 ml/min, and helium makeup gas set at 25 ml/min.

Gas chromatograms of the E. coli extracts are shown with the chain length of the ketones indicated in FIG. IX-1.

The complete disclosures of all patents, patent applications, publications, and nucleic acid and protein database entries, including for example GenBank accession numbers and EMBL accession numbers, that are cited herein are hereby incorporated by reference as if individually incorporated. Various modifications and alterations of this invention will become apparent to those skilled in the art without departing from the scope and spirit of this invention, and it should be understood that this invention is not to be unduly limited to the illustrative embodiments set forth herein.

What is claimed is:

1. A method of producing a ketone, the method comprising:
providing one or more fatty acids;
providing one or more modified cells and/or modified organisms that produce one or more OleA proteins;
providing conditions effective to produce the one or more OleA proteins; and
providing conditions effective to produce one or more ketones from said one or more fatty acids in the presence of the one or more OleA proteins.

2. A method of producing a beta-keto-acid, the method comprising:
providing one or more fatty acids;
providing one or more modified cells and/or modified organisms that produce one or more OleA proteins;
providing conditions effective to produce the one or more OleA proteins; and
providing conditions effective to produce one or more beta-keto-acids from said one or more fatty acids in the presence of the one or more OleA proteins.

3. A method of producing a ketone, the method comprising:
providing one or more fatty acids;
providing one or more isolated and purified OleA proteins; and
combining the fatty acids with the isolated and purified OleA proteins under conditions effective to produce one or more ketones from said one or more fatty acids.

4. A method of producing a beta-keto-acid, the method comprising:
providing one or more fatty acids;
providing one or more isolated and purified OleA proteins; and
combining the fatty acids with the isolated and purified OleA proteins under conditions effective to produce one or more beta-keto-acids from said one or more fatty acids.

5. A method of producing a hydrocarbon, the method comprising:
providing one or more modified cells and/or modified organisms that produce one or more fatty acids and produce at least one OleA protein, at least one OleC protein, and at least one OleD protein;
providing conditions effective to produce at least one OleA protein, at least one oleC protein, and at least one OleD protein; and
providing conditions effective to produce one or more hydrocarbons from said one or more fatty acids in the presence of at least one OleA protein, at least one OleC protein, and at least one OleD protein.

6. A method of producing a hydrocarbon, the method comprising: providing one or more fatty acids;
providing an isolated and purified OleA protein, an isolated and purified OleC protein, and an isolated and purified OleD protein; and
providing conditions effective to produce one or more hydrocarbons from said one or more fatty acids in the presence of at least one OleA protein, at least one OleC protein, and at least one OleD protein.

7. A modified bacterial organism that has altered hydrocarbon production relative to the wild-type bacterial organism.

8. A modified bacterial organism that has altered ketone production relative to a corresponding unmodified bacterial organism.

9. A method of modifying a bacterial organism to produce altered hydrocarbon production relative to the wild-type bacterial organism comprising:
removing genomic nucleic acid that encodes OleA, OleB, OleC, or OleD proteins; and
inserting nucleic acid that encodes a heterologous protein having fatty acyl condensase function.

10. A method of controlling the synthesis of a hydrocarbon, the method comprising:
providing a modified bacterial organism of claim 7; and
culturing the modified bacterial organism under conditions effective to produce one or more hydrocarbons.

11. A method of controlling the synthesis of a ketone, the method comprising:
providing a modified bacterial organism of claim 7; and
culturing the modified bacterial organism under conditions effective to produce one or more ketones.

12. A method of controlling the synthesis of an energy storage molecule, the method comprising:
providing a modified bacterial organism of claim 7; and
culturing the modified bacterial organism under conditions effective to produce one or more energy storage molecules.

13. A hydrocarbon mixture produced by the method of claim 10.

14. A ketone mixture produced by the method of claim 11.

15. An isolated and purified nucleic acid construct comprising nucleic acids encoding an oleA protein.

16. A vector comprising the isolated and purified nucleic acid of claim 15.

17. A cell comprising the vector of claim 16.

18. A method of extracting a mixture of ketones from a biological culture comprising:
providing a culture comprising a modified bacterial organism of claim 7;
growing the culture under conditions wherein said ketones are produced in said culture;
preparing an organic extract from said culture; and
purifying ketones from said extract;
thereby producing an extract containing a mixture of ketones.
